(19) United States
(12) Patent Application Publication
(10) Pub. No.: US 2025/0082722 A1
(43) Pub. Date: Mar. 13, 2025

(54) ACTRII PROTEINS FOR THE TREATMENT OF PULMONARY ARTERIAL HYPERTENSION (PAH)

(71) Applicant: Acceleron Pharma Inc., Cambridge, MA (US)

(72) Inventors: Janethe de Oliveira Pena, Cambridge, MA (US); Peter Linde, Cambridge, MA (US); Sachindra Joshi, Cambridge, MA (US); Gang Li, Cambridge, MA (US); Patrick Andre, Cambridge, MA (US); Ravindra Kumar, Cambridge, MA (US)

(21) Appl. No.: 18/954,145

(22) Filed: Nov. 20, 2024

Related U.S. Application Data
(63) Continuation of application No. 18/011,261, filed on Dec. 19, 2022.
(60) Provisional application No. 63/188,141, filed on May 13, 2021.
(60) Provisional application No. 63/112,513, filed on Nov. 11, 2020.
(60) Provisional application No. 63/084,409, filed on Sep. 28, 2020.
(60) Provisional application No. 63/042,722, filed on Jun. 23, 2020.

(51) Int. Cl. A61K 38/17; A61K 45/06; A61P 9/12

(52) U.S. Cl. CPC A61K 38/1709; A61K 45/06; A61P 9/12

(57) ABSTRACT

In some aspects, the disclosure relates to compositions and methods comprising ActRII polypeptides to treat, prevent, or reduce the progression rate and/or severity of pulmonary arterial hypertension, particularly treating, preventing or reducing the progression rate and/or severity of one or more pulmonary arterial hypertension associated complications.

### CROSS-REFERENCE TO RELATED APPLICATIONS

This application claims the benefit of priority to U.S. provisional application Ser. No. 63/042,722, filed on Jun. 23, 2020; 63/084,409, filed on Sep. 28, 2020; 63/112,513, filed on Nov. 11, 2020; and 63/188,141, filed on May 13, 2021. The disclosures of the foregoing applications are hereby incorporated by reference in their entirety.

### BACKGROUND OF THE INVENTION

Pulmonary hypertension (PH) is a disease characterized by high blood pressure in lung vasculature, including pulmonary arteries, pulmonary veins, and pulmonary capillaries. In general, PH is defined as a mean pulmonary artery pressure (mPAP) ≥20 mm Hg at rest or ≥30 mm Hg with exercise [Hill et al., Respiratory Care 54(7):958-68 (2009)]. One of the main PH symptoms is difficulty in breathing or shortness of breath, and other symptoms include fatigue, dizziness, fainting, peripheral edema (swelling in foot, legs or ankles), bluish lips and skin, chest pain, angina pectoris, light-headedness during exercise, non-productive cough, racing pulse and palpitations. PH can be a severe disease causing heart failure, which is one of the most common causes of death in people who have pulmonary hypertension. Postoperative pulmonary hypertension may complicate many types of surgeries or procedures, and present a challenge associated with a high mortality.

PH may be grouped based on different manifestations of the disease sharing similarities in pathophysiologic mechanisms, clinical presentation, and therapeutic approaches [Simonneau et al., JACC 54(1):S44-54 (2009)]. Clinical classification of PH was first proposed in 1973, and a recent updated clinical classification was endorsed by the World Health Organization (WHO) in 2018. According to the updated PH clinical classification, there are five main groups of PH: pulmonary arterial hypertension (PAH), characterized by a pulmonary artery wedge pressure (PAWP) ≥15 mm Hg; PH due to left heart disease (also known as pulmonary venous hypertension or congestive heart failure), characterized by a PAWP >15 mm Hg; PH due to lung diseases and/or hypoxia; PH due to pulmonary artery obstructions; and PH with unclear and/or multifactorial etiologies [Simonneau et al., JACC 54(1):S44-54 (2009); Hill et al., Respiratory Care 54(7):958-68 (2009)]. PAH is further classified into idiopathic PAH (IPAH), a sporadic disease in which there is neither a family history of PAH nor an identified risk factor; heritable PAH; PAH induced by drugs and toxins; PAH associated with connective tissue diseases, HIV infection, portal hypertension, congenital heart diseases, schistosomiasis, and chronic hemolytic anemia; and persistent PH of newborns [Simonneau et al., (2019) Eur Respir J: 53:1801913]. Diagnosis of various types of PH requires a series of tests.

In general, PH treatment depends on the cause or classification of PH. Where PH is caused by a known medicine or medical condition, it is known as a secondary PH, and its treatment is usually directed at the underlying disease. Treatment of Group 2 pulmonary hypertension (e.g., venous hypertension) generally involves optimizing left ventricular function by administering diuretics, beta blockers, and ACE inhibitors, or repairing or replacing a mitral valve or aortic valve. PAH therapies include pulmonary vasodilators, digoxin, diuretics, anticoagulants, and oxygen therapy. Pulmonary vasodilators target different pathways, including prostacyclin pathway (e.g., prostacyclins, including intravenous epoprostenol, subcutaneous or intravenous treprostinil, and inhaled iloprost), nitric oxide pathway (e.g., phosphodiesterase-5 inhibitors, including sildenafil and tadalafil), and endotheline-1 pathway (e.g., endothelin receptor antagonists, including oral bosentan and oral ambrisentan) [Humbert, M. Am. J. Respir. Crit. Care Med. 179:650-6 (2009); Hill et al., Respiratory Care 54(7):958-68 (2009)]. However, current therapies provide no cure for PH, and they do not directly treat the underling vascular remodeling and muscularization of blood vessels observed in many PH patients.

There is a high, unmet need for effective therapies for treating pulmonary hypertension. Accordingly, it is an object of the present disclosure to provide methods for treating, preventing, or reducing the progression rate and/or severity of PH, particularly treating, preventing or reducing the progression rate and/or severity of one or more PH-associated complications.

### SUMMARY OF THE INVENTION

In certain aspects, the disclosure provides for a method of treating pulmonary arterial hypertension (PAH), comprising administering a therapeutically effective amount of an ActRII polypeptide to a patient, wherein the polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the polypeptide is administered at a dosing range of 0.1 mg/kg to 2.0 mg/kg, and wherein administration of said polypeptide results in a change in one or more of the following hemodynamic or functional parameters: a reduction in pulmonary vascular resistance (PVR); an increase in 6-minute walk distance (6 MWD); a decrease of the N-terminal pro B-type natriuretic peptide (NT-proBNP) levels; the prevention or reduction of pulmonary hypertension Functional Class progression as recognized by the World Health Organization (WHO); the promotion or increasing of pulmonary hypertension Functional Class regression as recognized by the WHO; an improvement in right ventricular function; an improvement in pulmonary artery pressure; and/or an improvement in mean right atrial pressure.

In certain aspects, the disclosure provides for a method of treating, preventing, or reducing the progression rate and/or severity of one or more complications of pulmonary arterial hypertension, comprising administering to a patient in need thereof an effective amount of an ActRII polypeptide to a patient, wherein the polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the polypeptide is administered at a dosing range of 0.1 mg/kg to 2.0 mg/kg, and wherein administration of said polypeptide results in a change in one or more of the following hemodynamic or functional parameters: a reduction in pulmonary vascular resistance (PVR); an increase in 6-minute walk distance (6 MWD); a decrease of the N-terminal pro B-type natriuretic peptide (NT-proBNP) levels; the prevention or reduction in pulmonary hypertension Functional Class progression as recognized by the World Health Organization (WHO); the promotion or increase of pulmonary hypertension Functional Class regression as recognized by the WHO; an improvement in right ventricular function; an improvement in pulmonary artery pressure; and/or an improvement in mean right atrial pressure. In some embodiments, the one or more complications of pulmonary arterial hypertension is selected from the group consisting of: smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis.

In certain aspects, the disclosure provides for a method of treating pulmonary arterial hypertension (PAH), comprising administering a dosing regimen of therapeutically effective amount of an ActRII polypeptide to a patient, wherein the polypeptide comprises an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, comprising a first dose of between 0.1 mg/kg and 1.0 mg/kg of said polypeptide for a first period of time, and a second dose of between 0.1 mg/kg and 1.0 mg/kg of said polypeptide is subsequently administered for a second period of time. In some embodiments, the administration of said polypeptide results in a change in one or more of the following hemodynamic or functional parameters: reduction in pulmonary vascular resistance (PVR); increase in 6-minute walk distance (6 MWD); decrease of the N-terminal pro B-type natriuretic peptide (NT-proBNP) levels; prevents or reduces pulmonary hypertension Functional Class progression as recognized by the World Health Organization (WHO); promotes or increases pulmonary hypertension Functional Class regression as recognized by the WHO; improvement in right ventricular function; improvement in pulmonary artery pressure; and improvement in mean right atrial pressure. In some embodiments, the first period of time is at least 3 weeks. In some embodiments, the second period of time is at least 3 weeks. In some embodiments, the second period of time is at least 21 weeks. In some embodiments, the second period of time is at least 45 weeks. In some embodiments, the second period of time exceeds the first period of time. In some embodiments, the second dose exceeds the first dose. In some embodiments, the first dose is in the range of about 0.2 mg/kg to about 0.4 mg/kg followed by a second dose in the range of about 0.5 mg/kg to about 0.8 mg/kg. In some embodiments, the first dose is about 0.3 mg/kg followed by a second dose of about 0.7 mg/kg.

In some embodiments, the method reduces the PVR in the patient. In some embodiments, the method reduces the PVR in the patient by at least 10% (e.g., 10%, 15%, 20%, 25%, 30%, 35%, 40%, 45%, or at least 50%). In some embodiments, the method reduces the patient's PVR by at least 20%. In some embodiments, the reduction in PVR is a result of decreased mean pulmonary artery pressure. In some embodiments, the method increases the patient's 6-minute walk distance. In some embodiments, the method increases the patient's 6-minute walk distance by at least 10 meters (e.g., at least 10, 20, 30, 40, 50, 60, 70, 80, 90, 100, 125, 150, 175, 200, 250, 300, or more than 400 meters). In some embodiments, the method increases the patient's 6-minute walk distance by at least 30 meters. In some embodiments, the method decreases NT-proBNP levels in the patient. In some embodiments, the method decreases NT-proBNP levels in the patient by at least 10% (e.g., 10%, 15%, 20%, 25%, 30%, 35%, 40%, 45%, 50%, 55%, 60%, 65%, 70%, 75%, or at least 80%). In some embodiments, the method decreases NT-proBNP levels in the patient by at least 30%. In some embodiments, the method decreases NT-proBNP levels to normal levels. In some embodiments, the normal level of NT-proBNP is <100 μg/ml.

In some embodiments, the method prevents or reduces pulmonary hypertension Functional Class progression as recognized by the WHO. In some embodiments, the method prevents or reduces pulmonary hypertension Functional Class progression from Functional Class I to Class II pulmonary hypertension as recognized by the WHO. In some embodiments, the method prevents or reduces pulmonary hypertension Functional Class progression from Functional Class II to Class III pulmonary hypertension as recognized by the WHO. In some embodiments, the method prevents or reduces pulmonary hypertension Functional Class progression from Functional Class III to Class IV pulmonary hypertension as recognized by the WHO. In some embodiments, the method promotes or increases pulmonary hypertension Functional Class regression as recognized by the WHO. In some embodiments, the method promotes or increases pulmonary hypertension Functional Class regression from Class IV to Class III pulmonary hypertension as recognized by the WHO. In some embodiments, the method promotes or increases pulmonary hypertension Functional Class regression from Class III to Class II pulmonary hypertension as recognized by the WHO. In some embodiments, the method promotes or increases pulmonary hypertension Functional Class regression from Class II to Class I pulmonary hypertension as recognized by the WHO.

In some embodiments, the method improves right ventricular function in the patient. In some embodiments, the improvement in right ventricular function is due to an increase in right ventricular fractional area change. In some embodiments, the improvement in right ventricular function is due to a decrease in right ventricular hypertrophy. In some embodiments, the improvement in right ventricular function is due to an increase in ejection fraction. In some embodiments, the improvement in right ventricular function is due to an increase in right ventricular fractional area change and ejection fraction.

In some embodiments, the method improves the pulmonary artery pressure in the patient. In some embodiments, the improvement in pulmonary artery pressure is a reduction in the mean pulmonary artery pressure (mPAP). In some embodiments, the method reduces the mPAP in the patient by at least 10% (e.g., 10%, 15%, 20%, 25%, 30%, 35%, 40%, 45%, or at least 50%). In some embodiments, the method reduces the mPAP by at least 3 mmHg (e.g., at least 3, 5, 7, 10, 12, 15, 20, or 25 mmHg) in the patient. In some embodiments, the method improves the mean right atrial pressure (mRAP) in the patient. In some embodiments, the improvement in the mRAP is a reduction in the mRAP. In some embodiments, the method reduces the mRAP in the patient by at least 10% (e.g., 10%, 15%, 20%, 25%, 30%, 35%, 40%, 45%, or at least 50%). In some embodiments, the method reduces the mRAP by at least 1 mmHg (e.g., at least 1, 2, 3, 4, 5, 6, 7, 8, 9, 10, 11, 12, 13, 14, or 15 mmHg) in the patient.

In some embodiments, the patient has a pulmonary vascular resistance (PVR) greater than or equal to 3 Wood Units. In some embodiments, the patient has a 6-minute walk distance from 150 to 550 meters. In some embodiments, the patient has elevated NT-proBNP levels as compared to a healthy patient. In some embodiments, the patient has a NT-proBNP level of at least 100 pg/mL (e.g., 100, 150, 200, 300, 400, 500, 1000, 3000, 5000, 10,000, 15,000, or 20,000 pg/mL). In some embodiments, the patient has elevated brain natriuretic peptide (BNP) levels as compared to a healthy patient. In some embodiments, the patient has a BNP level of at least 100 pg/mL (e.g., 100, 150, 200, 300, 400, 500, 1000, 3000, 5000, 10,000, 15,000, or 20,000 pg/mL). In some embodiments, the method decreases BNP levels in the patient by at least 10% (e.g., 10%, 15%, 20%, 25%, 30%, 35%, 40%, 45%, 50%, 55%, 60%, 65%, 70%, 75%, or at least 80%). In some embodiments, the method decreases BNP levels to normal levels (i.e., <100 μg/ml). In some embodiments, the patient has a mean pulmonary artery pressure (mPAP) selected from the group consisting of: an mPAP of at least 20 mmHg; an mPAP of at least 25 mmHg; an mPAP of at least 30 mmHg; an mPAP of at least 35 mmHg; an mPAP of at least 40 mmHg; an mPAP of at least 45 mmHg; and an mPAP of at least 50 mmHg. In some embodiments, the patient has a mean right atrial pressure (mRAP) selected from the group consisting of: an mRAP of at least 5 mmHg; an mRAP of at least 6 mmHg; an mRAP of at least 8 mmHg; an mRAP of at least 10 mmHg; an mRAP of at least 12 mmHg; an mRAP of at least 14 mmHg; and an mRAP of at least 16 mmHg.

In some embodiments, the PAH is idiopathic pulmonary arterial hypertension (PAH). In some embodiments, the PAH is heritable PAH. In some embodiments, the PAH is drug- or toxin-induced PAH. In some embodiments, the PAH is PAH associated with simple, congenital systemic-to-pulmonary shunts at least 1 year following shunt repair. In some embodiments, the patient has Functional Class II or Class III pulmonary hypertension in accordance with the World Health Organization's functional classification system for pulmonary hypertension. In some embodiments, the patient has Functional Class I, Class II, Class III, or Class IV pulmonary hypertension as recognized by the World Health Organization. In some embodiments, the patient has Functional Class I, Class II, Class III, or Class IV pulmonary hypertension in accordance with the World Health Organization's functional classification system for pulmonary hypertension. In some embodiments, the patient has Functional Class IV pulmonary hypertension in accordance with the World Health Organization's functional classification system for pulmonary hypertension. In some embodiments, the method increases transplant free survival in the patient. In some embodiments, the method increases transplant free survival in the patient by at least 10% (e.g., 10%, 15%, 20%, 25%, 30%, 35%, 40%, 45%, or at least 50%). In some embodiments, the method decreases right ventricular hypertrophy in the patient. In some embodiments, the method decreases right ventricular hypertrophy in the patient by at least 10% (e.g., 10%, 15%, 20%, 25%, 30%, 35%, 40%, 45%, or at least 50%). In some embodiments, the method decreases smooth muscle hypertrophy in the patient. In some embodiments, the method decreases smooth muscle hypertrophy in the patient by at least 10% (e.g., 10%, 15%, 20%, 25%, 30%, 35%, 40%, 45%, or at least 50%). In some embodiments, the method decreases pulmonary arteriole muscularity in the patient. In some embodiments, the method decreases pulmonary arteriole muscularity in the patient by at least 10% (e.g., 10%, 15%, 20%, 25%, 30%, 35%, 40%, 45%, or at least 50%).

In some embodiments, the method increases exercise capacity of the patient. In some embodiments, the method reduces the patient's Borg dyspnea index (BDI). In some embodiments, the method reduces the patient's BDI by at least 0.5 index points (e.g., at least 0.5, 1, 1.5, 2, 2.5, 3, 3.5, 4, 4.5, 5, 5.5, 6, 6.5, 7, 7.5, 8, 8.5, 9, 9.5, or 10 index points). In some embodiments, the patient has decreased renal function. In some embodiments, the method further improves renal function. In some embodiments, the method delays clinical worsening of pulmonary arterial hypertension. In some embodiments, the method delays clinical worsening of pulmonary arterial hypertension in accordance with the World Health Organization's functional classification system for pulmonary hypertension. In some embodiments, the method reduces the risk of hospitalization for one or more complications associated with pulmonary arterial hypertension. In some embodiments, the method reduces the risk of morbidity for one or more complications associated with pulmonary arterial hypertension. In some embodiments, the morbidity comprises a change in one or more of the following: increased need for a lung and/or heart transplant; need to initiate rescue therapy with a known treatment for PAH; need to increase prostacyclin by at least 10%; need for atrial septostomy; PAH-specific hospitalization for at least 24 hours; and deterioration of PAH. In some embodiments, the deterioration of PAH comprises a worsening in WHO functional class and a decrease in 6 MWD of at least 15%. In some embodiments, the method reduces the risk of death associated with pulmonary arterial hypertension. In some embodiments, the method reduces the risk of death associated with pulmonary arterial hypertension by at least 10% (e.g., 10%, 15%, 20%, 25%, 30%, 35%, 40%, 45%, or at least 50%). In some embodiments, the patient has a hemoglobin level from >8 and <15 g/dl. In some embodiments, the patient's hemoglobin levels are <18 g/dl.

In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the sequence of amino acids corresponding to residues 30-110 of SEQ ID NO: 1. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 2. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3. In some embodiments, the ActRII polypeptide is a fusion protein further comprising an Fc domain of an immunoglobulin. In some embodiments, the Fc domain of the immunoglobulin is an Fc domain of an IgG1 immunoglobulin. In some embodiments, the Fc fusion protein further comprises a linker domain positioned between the ActRII polypeptide domain and the Fc domain of the immunoglobulin. In some embodiments, the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21). In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 23. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 41. In some embodiments, the polypeptide comprises an amino acid sequence that is at least 90% identical to an amino acid sequence corresponding to residues 30-110 of SEQ ID NO: 1. In some embodiments, the polypeptide comprises an amino acid sequence that is at least 90% identical to an amino acid sequence corresponding to residues 21-135 of SEQ ID NO: 1. In some embodiments, the polypeptide is lyophilized. In some embodiments, the polypeptide is soluble. In some embodiments, the polypeptide is administered to the patient using subcutaneous injection. In some embodiments, the polypeptide is administered to the patient every 3 weeks. In some embodiments, the polypeptide is administered to the patient every 4 weeks. In some embodiments, the ActRII polypeptide is administered to the patient every week, every two weeks, every three weeks, or every four weeks. In some embodiments, the ActRII polypeptide is administered to the patient every three weeks. In some embodiments, the polypeptide is part of a homodimer protein complex. In some embodiments, the polypeptide is glycosylated. In some embodiments, the polypeptide has a glycosylation pattern obtainable by expression in a Chinese hamster ovary cell. In some embodiments, the ActRII polypeptide binds to one or more ligands selected from the group consisting of: activin A, activin B, and GDF11. In some embodiments, the ActRII polypeptide binds to activin and/or GDF11. In some embodiments, the ActRII polypeptide further binds to one or more ligands selected from the group consisting of: BMP10, GDF8, and BMP6.

In some embodiments, the ActRII polypeptide is administered at a dose between 0.1 mg/kg and 2.0 mg/kg. In some embodiments, the ActRII polypeptide is administered at a dose of 0.3 mg/kg. In some embodiments, the ActRII polypeptide is administered at a dose of 0.7 mg/kg. In some embodiments, the method further comprises administering to the patient an additional active agent and/or supportive therapy. In some embodiments, the additional active agent and/or supportive therapy is selected from the group consisting of: beta-blockers, angiotensin-converting enzyme inhibitors (ACE inhibitors), angiotensin receptor blockers (ARBs), diuretic agents, lipid-lowering medications, endothelin blockers, PDE5 inhibitors, prostacyclins, or a left ventricular assist device (LVAD). In some embodiments, the additional active agent and/or supportive therapy is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1->3)-beta-D-glucopyranosyl]oleanolic acid; 3-O-[beta-D-glucopyranosyl-(1->2)-beta-D-glucopyranosyl]oleanolic acid; 3-O-[beta-D-glucopyranosyl-(1->3)-beta-D-glucopyranosyl]oleanolic acid 28-O-beta-D-glucopyranosyl ester; 3-O-[beta-D-glucopyranosyl-(1->2)-beta-D-glucopyranosyl]oleanolic acid 28-O-beta-D-glucopyranosyl ester; 3-O-[a-L-rhamnopyranosyl-(1->3)-beta-D-glucuronopyranosyl]oleanolic acid; 3-O-[alpha-L-rhamnopyranosyl-(1->3)-beta-D-glucuronopyranosyl]oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate); a left ventricular assist device (LVAD), or lung and/or heart transplantation. In some embodiments, the patient has been treated with one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil. In some embodiments, the method further comprises administration of one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil. In some embodiments, the patient has been treated with one or more vasodilators prior to administration of the polypeptide. In some embodiments, the method further comprises administration of one or more vasodilators. In some embodiments, the one or more vasodilators is selected from the group consisting of prostacyclin, epoprostenol, and sildenafil. In some embodiments, the vasodilator is prostacyclin.

In some embodiments, the patient has been receiving one or more therapies for PAH. In some embodiments, the one or more therapies for PAH is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1->3)-beta-D-glucopyranosyl]oleanolic acid; 3-0-[beta-D-glucopyranosyl-(1->2)-beta-D-glucopyranosyl]oleanolic acid; 3-O-[beta-D-glucopyranosyl-(1->3)-beta-D-glucopyranosyl]oleanolic acid 28-O-beta-D-glucopyranosyl ester; 3-O-[beta-D-glucopyranosyl-(1->2)-beta-D-glucopyranosyl]oleanolic acid 28-O-beta-D-glucopyranosyl ester; 3-O-[a-L-rhamnopyranosyl-(1->3)-beta-D-glucuronopyranosyl]oleanolic acid; 3-O-[alpha-L-rhamnopyranosyl-(1->3)-beta-D-glucuronopyranosyl]oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2.3]oleanolate); a left ventricular assist device (LVAD), or lung and/or heart transplantation.

In certain aspects, the disclosure provides for a method of treating or preventing cardiopulmonary remodeling associated with pulmonary arterial hypertension in a patient, comprising administering to a patient in need thereof an effective amount of an ActRII polypeptide, wherein said method slows down cardiac remodeling and/or reverses cardiac remodeling. In some embodiments, the reversal in cardiac remodeling is a sustained reversal. In some embodiments, the cardiopulmonary remodeling is ventricle remodeling. In some embodiments, the ventricle remodeling is left ventricular remodeling. In some embodiments, the ventricle remodeling is right ventricular remodeling. In some embodiments, the cardiopulmonary remodeling is ventricular dilation.

In certain aspects, the disclosure provides for a kit comprising a lyophilized polypeptide and an injection device, wherein the polypeptide is an ActRII polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the polypeptide is a polypeptide comprising an amino acid sequence that is at least 90% identical to an amino acid sequence corresponding to residues 30-110 of SEQ ID NO: 1. In some embodiments, the polypeptide is a polypeptide comprising an amino acid sequence that is at least 95% identical to the amino acid sequence corresponding to residues 30-110 of SEQ ID NO: 1. In some embodiments, the polypeptide is a polypeptide comprising an amino acid sequence that is at least 99% identical to the amino acid sequence corresponding to residues 30-110 of SEQ ID NO: 1. In some embodiments, the polypeptide is a polypeptide comprising the amino acid sequence corresponding to residues 30-110 of SEQ ID NO: 1. In some embodiments, the polypeptide is a polypeptide consisting of the amino acid sequence corresponding to residues 30-110 of SEQ ID NO: 1. In some embodiments, the polypeptide is a polypeptide comprising an amino acid sequence that is at least 90% identical to the amino acid sequence corresponding to residues 21-135 of SEQ ID NO: 1. In some embodiments, the polypeptide is a polypeptide comprising an amino acid sequence that is at least 95% identical to the amino acid sequence corresponding to residues 21-135 of SEQ ID NO: 1. In some embodiments, the polypeptide is a polypeptide comprising an amino acid sequence that is at least 99% identical to the amino acid sequence corresponding to residues 21-135 of SEQ ID NO: 1. In some embodiments, the polypeptide is a polypeptide comprising the amino acid sequence corresponding to residues 21-135 of SEQ ID NO: 1. In some embodiments, the polypeptide is a polypeptide consisting of the amino acid sequence corresponding to residues 21-135 of SEQ ID NO: 1. In some embodiments, the polypeptide is a polypeptide comprising an amino acid sequence that is at least 90% identical to the amino acid sequence of SEQ ID NO: 2. In some embodiments, the polypeptide is a polypeptide comprising an amino acid sequence that is at least 95% identical to the amino acid sequence of SEQ ID NO: 2. In some embodiments, the polypeptide is a polypeptide comprising an amino acid sequence that is at least 99% identical to the amino acid sequence of SEQ ID NO: 2. In some embodiments, the polypeptide is a polypeptide comprising the amino acid sequence of SEQ ID NO: 2. In some embodiments, the polypeptide is a polypeptide consisting of the amino acid sequence of SEQ ID NO: 2. In some embodiments, the polypeptide is a polypeptide comprising an amino acid sequence that is at least 90% identical to the amino acid sequence of SEQ ID NO: 3. In some embodiments, the polypeptide is a polypeptide comprising an amino acid sequence that is at least 95% identical to the amino acid sequence of SEQ ID NO: 3. In some embodiments, the polypeptide is a polypeptide comprising an amino acid sequence that is at least 99% identical to the amino acid sequence of SEQ ID NO: 3. In some embodiments, the polypeptide is a polypeptide comprising the amino acid sequence of SEQ ID NO: 3. In some embodiments, the polypeptide is a polypeptide consisting of the amino acid sequence of SEQ ID NO: 3. In some embodiments, the polypeptide is a fusion protein further comprising an Fc domain of an immunoglobulin. In some embodiments, the Fc domain of the immunoglobulin is an Fc domain of an IgG1 immunoglobulin. In some embodiments, the fusion protein further comprises a linker domain positioned between the polypeptide domain and the Fc domain of the immunoglobulin. In some embodiments, the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21). In some embodiments, the linker domain comprises TGGG (SEQ ID NO: 20). In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 23. In some embodiments, the ActRII polypeptide comprises the amino acid sequence of SEQ ID NO: 23. In some embodiments, the ActRII polypeptide consists of the amino acid sequence of SEQ ID NO: 23. In some embodiments, the polypeptide is part of a homodimer protein complex. In some embodiments, the polypeptide is glycosylated. In some embodiments, the polypeptide binds to one or more ligands selected from the group consisting of: activin A, activin B, and GDF11. In some embodiments, the polypeptide further binds to one or more ligands selected from the group consisting of: BMP10, GDF8, and BMP6. In some embodiments, the polypeptide binds to activin and/or GDF11.

In some embodiments, the kit comprises one or more vials containing the lyophilized polypeptide. In some embodiments, the kit comprises at least two vials containing the lyophilized polypeptide. In some embodiments, the two vials can contain the same or different amounts of the lyophilized polypeptide. In some embodiments, the vials comprise between 25 mg to 60 mg of the lyophilized polypeptide. In some embodiments, at least one of the vials contains 60 mg of lyophilized polypeptide. In some embodiments, at least one of the vials contains 45 mg of lyophilized polypeptide. In some embodiments, at least one of the vials contains 30 mg of lyophilized polypeptide. In some embodiments, at least one of the vials contains 25 mg of lyophilized polypeptide. In some embodiments, a first vial contains 45 mg of lyophilized polypeptide and a second vial contains 60 mg of lyophilized polypeptide. In some embodiments, a first vial contains 30 mg of lyophilized polypeptide and a second vial contains 60 mg of lyophilized polypeptide. In some embodiments, a first vial contains 45 mg of lyophilized polypeptide and a second vial contains 45 mg of lyophilized polypeptide. In some embodiments, a first vial contains 30 mg of lyophilized polypeptide, a second vial contains 45 mg of lyophilized polypeptide, and a third vial contains 60 mg of lyophilized polypeptide. In some embodiments, a first vial contains 25 mg of lyophilized polypeptide, a second vial contains 45 mg of lyophilized polypeptide, and a third vial contains 60 mg of lyophilized polypeptide. In some embodiments, the vials are refrigerated at 2-8° C.

In some embodiments, the injection device comprises a pre-filled syringe. In some embodiments, the injection device comprises a pump apparatus. In some embodiments, the pump apparatus comprises an electromechanical pumping assembly. In some embodiments, the pump apparatus is a wearable pump apparatus. In some embodiments, the pre-filled syringe comprises a reconstitution solution. In some embodiments, the reconstitution solution comprises a pharmaceutically acceptable carrier and/or excipient. In some embodiments, the pharmaceutically acceptable carrier is selected from saline solution, purified water, or sterile water for injection. In some embodiments, the pharmaceutically acceptable excipient is selected from a buffering agent [e.g., citric acid (monohydrate) and/or trisodium citrate (dehydrate)], a surfactant (e.g., polysorbate 80), a stabilizer (e.g., sucrose), and a lyoprotectant (e.g., sucrose). In some embodiments, the injection device comprises a vial adapter. In some embodiments, the vial adapter is capable of attaching to a vial. In some embodiments, the vial adapter is capable of attaching to a pre-filled syringe. In some embodiments, the pre-filled syringe and the vial are attached to opposite ends of the vial adapter. In some embodiments, the reconstitution solution is transferred from the pre-filled syringe to the vial. In some embodiments, the lyophilized polypeptide is reconstituted into a sterile injectable solution. In some embodiments, the lyophilized polypeptide is reconstituted into a sterile injectable solution prior to use. In some embodiments, the sterile injectable solution is sterile water for injection. In some embodiments, the sterile injectable solution is administered parenterally. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the injection device is used to administer the sterile injectable solution. In some embodiments, the sterile injectable solution comprises a therapeutically effective dose. In some embodiments, the therapeutically effective dose comprises a weight based dose. In some embodiments, the lyophilized polypeptide is administered every 3 weeks. In some embodiments, the lyophilized polypeptide is administered every 4 weeks. In some embodiments, the kit is used to treat PAH. In some embodiments, the shelf life of the lyophilized polypeptide is at least 1, 3, 6, 9, or 11 months. In some embodiments, the shelf life of the lyophilized polypeptide is at least 1, 1.5, 2, 2.5, or 3 years. In some embodiments, the lyophilized polypeptide is reconstituted. In some embodiments, the reconstituted polypeptide has a shelf life of at least 2 hours, 3 hours, or 4 hours.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1 shows an alignment of extracellular domains of human ActRIIB (SEQ ID NO: 31) and human ActRIIA (SEQ ID NO: 2) with the residues that are deduced herein, based on composite analysis of multiple ActRIIB and ActRIIA crystal structures, to directly contact ligand indicated with boxes.

FIG. 2 shows a multiple sequence alignment of various vertebrate ActRIIA proteins and human ActRIIA (SEQ ID NOs: 6-10 and 36-38).

FIG. 3 shows multiple sequence alignment of Fc domains from human IgG isotypes using Clustal 2.1. Hinge regions are indicated by dotted underline. Double underline indicates examples of positions engineered in IgG1 Fc (SEQ ID NO: 32) to promote asymmetric chain pairing and the corresponding positions with respect to other isotypes IgG2 (SEQ ID NO: 33), IgG3 (SEQ ID NO: 34) and IgG4 (SEQ ID NO: 35).

FIGS. 4A and 4B show the purification of ActRIIA-hFc expressed in CHO cells. The protein purifies as a single, well-defined peak as visualized by sizing column (FIG. 4A) and Coomassie stained SDS-PAGE (FIG. 4B) (left lane: molecular weight standards; right lane: ActRIIA-hFc).

FIGS. 5A and 5B show the binding of ActRIIA-hFc to activin (FIG. 5A) and GDF-11 (FIG. 5B), as measured by Biacore™ assay.

FIG. 6 shows the effects of vehicle, sildenafil, and ActRIIA-mFc treatment on vessel muscularity in a monocrotaline rat model of pulmonary arterial hypertension.

FIG. 7 shows the effects of vehicle, sildenafil, and ActRIIA-mFc treatment on vessel muscularity in a Sugen Hypoxia rat model of pulmonary arterial hypertension.

FIGS. 8A-8D show changes in pulmonary vascular resistance from baseline to week 24 in a placebo-controlled trial using sotatercept (an ActRIIA-hFc polypeptide) treatment in patients with pulmonary arterial hypertension. FIG. 8A shows the least squares mean±SE in the full analysis set. The least squares mean difference in pulmonary vascular resistance compared with placebo was −145.8 dyn·s/cm5 (95% CI, −241.0 to −50.6) for sotatercept 0.3 mg/kg, and −239.5 dyn·s/cm5 (95% CI, −329.3, −149.7) for sotatercept 0.7 mg/kg. FIG. 8B change in pulmonary vascular resistance by visit between baseline and end of placebo-controlled treatment period (week 24) in the full analysis set for sotatercept 0.3 mg/kg and 0.7 mg/kg groups ±SE. FIG. 8C shows the effect of sotatercept 0.3 mg/kg on the change in pulmonary vascular resistance from baseline to week 24 in patient subgroups. All data are from the full analysis set and compared with placebo using analysis of covariance with baseline values as the covariate. FIG. 8D shows the effect of sotatercept 0.7 mg/kg on the change in pulmonary vascular resistance from baseline to week 24 in patient subgroups. All data are from the full analysis set and compared with placebo using analysis of covariance with baseline values as the covariate.

FIGS. 9A and 9B show the change in various parameters from baseline to week 24 in a placebo-controlled trial using sotatercept (an ActRIIA-hFc polypeptide) treatment in patients with pulmonary arterial hypertension. Patients were treated with placebo, sotatercept 0.3 mg/kg, or sotatercept 0.7 mg/kg.

FIGS. 10A-C shows the change in the 6-Minute Walk Distance from baseline to week 24 in a placebo-controlled trial using sotatercept (an ActRIIA-hFc polypeptide) treatment in patients with pulmonary arterial hypertension. FIG. 10A shows the least squares mean±SE in the full analysis set. The least squares mean difference in 6-minute walk distance compared with placebo was 29.4 m (95% CI, 3.8 to 55.0) for sotatercept 0.3 mg/kg, and 21.4 m (95% CI, −2.8 to 45.7) for sotatercept 0.7 mg/kg. FIG. 10B shows the effect of sotatercept 0.3 mg/kg on the change in 6-Minute Walk Distance from baseline to week 24 in patient subgroups. All data are from the full analysis set and compared with placebo using analysis of covariance with baseline values as the covariate. FIG. 10C shows the effect of sotatercept 0.7 mg/kg on the change in 6-Minute Walk Distance from baseline to week 24 in patient subgroups. All data are from the full analysis set and compared with placebo using analysis of covariance with baseline values as the covariate.

FIGS. 11A-C shows the change in NT-proBNP from baseline to week 24 in a placebo-controlled trial using sotatercept (an ActRIIA-hFc polypeptide) treatment in patients with pulmonary arterial hypertension. FIG. 11A shows the least squares mean±SE in the full analysis set. The least squares mean difference in NT-ProBNP compared with placebo was −931.5 pg/mL (95% CI, −1353.24 to −5.0.70) for sotatercept 0.3 mg/kg, and −651.0 pg/mL (95% CI, −1043.28 to −258.74) for sotatercept 0.7 mg/kg. FIG. 11B shows show the effect of sotatercept 0.3 mg/kg on the change in NT-proBNP from baseline to week 24 in patient subgroups. All data are from the full analysis set and compared with placebo using analysis of covariance with baseline values as the covariate. FIG. 11C shows show the effect of sotatercept 0.7 mg/kg on the change in NT-proBNP from baseline to week 24 in patient subgroups. All data are from the full analysis set and compared with placebo using analysis of covariance with baseline values as the covariate.

FIG. 12 shows the mean change from baseline to week 24 in echocardiography parameters measured during a placebo-controlled trial using sotatercept (an ActRIIA-hFc polypeptide) treatment in patients with pulmonary arterial hypertension. Baseline data are mean (SD) and Change are LS Mean (SE) from the evaluable analysis set. All echocardiography data was obtained in 2D. TAPSE: tricuspid annular plane systolic excursion; RVFAC: right ventricular fractional area change. Week 24 includes end-of-treatment visit if the subject discontinued prior to the week 24 visit. P values are based on ANCOVA analysis using baseline WHO functional class and baseline result as covariates.

FIGS. 13A-F show that sotatercept analog RAP-011 (an ActRIIA-mFc polypeptide) prevents PH and reduces right ventricular hypertrophy in a mouse model of BMPR2 deficiency. Experimental strategy used to test preventive effects of RAP-011 in mice with Bmpr2 haploinsufficiency. Bmpr2+/R899X mice were exposed to normobaric hypoxia (FIO2=0.10) and treated twice-weekly with either RAP-011 (10 mg/kg, s.c.) or vehicle (PBS) for 5 weeks (FIG. 13A). FIG. 13B shows the right ventricular systolic pressure (RVSP) and FIG. 13C shows the Fulton index, calculated as the ratio of right ventricular weight (RV) to weight of the combined left ventricle and septum (LV+S). Data are means±SEM (n=7-10 per group). Analysis by one-way ANOVA and Tukey post hoc test. (FIG. 13D shows that amplification of genomic DNA by PCR and direct sequencing confirmed the presence of a heterozygous mutation (arrow) in Bmpr2+/R899X mice (equal peak heights for wild-type and mutant alleles). FIG. 13E shows an immunoblot of lung homogenates from wild-type and Bmpr2+/R899X mice analyzed to determine expression of BMPR2. FIG. 13F shows quantification of BMPR2 protein expression normalized to GADPH. Data are means±SEM (n=5 per group). Analysis by Students t-test. *P<0.05, ***P<0.001, ****P<0.0001.

FIGS. 14A-E show that RAP-011 is effective in combination therapy as well as monotherapy for reversing pulmonary vascular remodeling in severe experimental PAH. FIG. 14A shows the experimental strategy used to test therapeutic effects of RAP-011 in a Sugen-hypoxia-normoxia (SuHxNx) rat model of severe PAH. Rats were treated on day 0 with a single dose of SU5416 (20 mg/kg) and exposed to normobaric hypoxia (FIO2=0.10) for 3 weeks followed by 6 weeks of normoxia to allow disease progression. Rats were additionally treated with RAP-011 (2.5 mg/kg, s.c., twice weekly), sildenafil (30 mg/kg, p.o., twice daily), combination therapy with RAP-011 and sildenafil, or vehicle (PBS) for 4 weeks starting on week 5 post SU5416. FIG. 14B shows the RVSP and FIG. 14C shows the total pulmonary resistance index (TPRI). Data are means±SEM (n=7-14 per group). FIG. 14D shows images of representative lung sections stained with hematoxylin and eosin. Scale bar, 200 μm. FIG. 14E shows images of lung sections immunostained with an antibody against α-smooth muscle actin to illustrate grades of pulmonary histopathology. Scale bar, 50 μm. Percentage of pulmonary arterial vessels classified as grade 0 (normal, no occlusion), grade 1 (<50% occlusion), or grade 2 (>50% occlusion) grouped according to vessel outer diameter. Data are means±SEM (n=4 rats per group). Analysis by one-way ANOVA and Tukey post hoc test; for simplicity, only significance for percentage of grade 0 vessels is indicated (*P<0.05).

FIGS. 15A-G show that concurrent inhibition of activins, GDF8, and GDF11 contributes to effects of RAP-011 in PH models in vitro and in vivo. FIG. 15A shows the effect of therapeutic treatment with RAP-011 on pulmonary cell proliferation in the SuHxNx model of severe PAH as measured by percentage of cells positive for Ki67. Data are means±SEM (n=4-5 rats per group). FIG. 15B illustrates the cell culture system used to investigate antiproliferative action of sotatercept. Human pulmonary artery smooth muscle cells (PASMCs) were treated with conditioned medium collected from human pulmonary artery endothelial cells PAECs in the absence or presence of separate antibodies against activin A and activin B (anti-Act), a dual antibody against GDF8 and GDF11 (anti-GDF), combined anti-Act and anti-GDF, or sotatercept (ACE-011). PASMC proliferation was quantified in a bromodeoxyuridine (BrdU) assay as shown in FIG. 15C. Data are means±SEM (n=8 per group). FIG. 15D illustrates the experimental strategy used to test preventive effects of multi-ligand inhibition in a SuHx rat model of PH. Rats were treated with a single dose of SU5416 (20 mg/kg, s.c.), exposed to normobaric hypoxia (FIO2=0.13), and treated s.c. twice weekly with anti-Act (10 mg/kg+10 mg/kg), anti-GDF (10 mg/kg), combined anti-Act and anti-GDF, or vehicle (PBS) for 4 weeks starting 1 day post SU5416. FIG. 15E shows the systolic pulmonary artery pressure (sPAP) in the rats treated according to FIG. 15D. FIG. 15F shows the mPAP in rats treated according to FIG. 15D. FIG. 15G shows the Fulton index in rats treated according to FIG. 15D. Data are means±SEM (n=5-9 rats per group). Analysis by one-way ANOVA and Tukey post hoc test (*P<0.05, **P<0.01, ****P<0.0001).

FIGS. 16A-K show that therapeutic treatment with RAP-011, but not sildenafil, reduces cardiac hypertrophy, restores septal wall geometry, and improves right ventricular function in severe experimental PAH. FIG. 16A shows the Fulton index and FIG. 16B shows the cardiac index (CI) in normal or SuHxNx rats as a function of treatment. Data are means±SEM (n=7-13 rats per group). FIG. 16C shows representative echocardiographic images obtained in a repeated manner from individual SuHxNx rats before and after therapy. FIG. 16D shows the pulmonary artery acceleration time (PAAT) in animals treated according to Example 13. FIG. 16E shows tricuspid annular plane systolic excursion (TAPSE) in animals treated according to Example 13. FIG. 16F shows the right ventricular wall thickness (RVWT) measured at diastole in animals treated according to Example 13. FIG. 16G shows the right ventricular fractional area change (RVFAC) in animals treated according to Example 13. Data are means±SEM (n=7-11 rats per group). FIGS. 16H-K shows the ratio of myosin heavy-chain isoform expression (Myh7:Myh6) (FIG. 16H) and levels of Nppb (FIG. 16I), Inhba (FIG. 16J), and Inhbb (FIG. 16K) mRNA in the right ventricle of normal or SuHxNx rats as a function of treatment. Data are means±SEM (n=6-11 rats per group). Analysis by one-way ANOVA and Tukey post hoc test (*P<0.05, **P<0.01, ***P<0.001, ****P<0.0001).

FIGS. 17A-I show RAP-011 exerts structural and functional cardioprotective effects in a model of right heart failure caused by pressure overload. FIG. 17A shows the experimental strategy used to assess potential cardioprotective effects of RAP-011 in a mouse model of sustained pressure overload. Wild-type mice were subjected to pulmonary artery banding and treated twice weekly with either RAP-011 (10 mg/kg, s.c.) or vehicle (PBS) for 3 weeks starting 1 day post-surgery. Various parameters measured using the experimental strategy described in FIG. 17A are as follows: Fulton index (FIG. 17B), right ventricular free wall thickness (RVFWT) (FIG. 17C), TAPSE (FIG. 17D), myocardial performance index (MPI) (FIG. 17E), right ventricular developed pressure (RVDP) (FIG. 17F), and peak rates of right ventricular pressure rise (dP/dtmax) and decline (−dP/dtmin) (FIG. 17G). Data are means±SEM (n=10-15 mice per group for day 21). FIG. 17H shows representative images of right ventricle sections stained with Masson's trichrome blue to detect fibrosis (scale bar, 20 μm), and FIG. 17I shows the quantification of percentage area occupied by fibrotic tissue. Data are means±SEM (n=10-15 mice per group). Analysis by one-way ANOVA and Tukey post hoc test (*p<0.05, **p<0.01, ***p<0.001, ****p<0.0001).

FIGS. 18A-G show the disease-reversing effects of RAP-011 in severe experimental PAH persist after treatment withdrawal. FIG. 18A shows the experimental strategy used to test the persistence of therapeutic effects of RAP-011 in a SuHxNx rat model of severe PAH. Rats were treated on day 0 with a single dose of SU5416 (20 mg/kg, s.c.) and exposed to 3 weeks of normobaric hypoxia (FIO2=0.10) followed by 10 weeks of normoxia to allow disease progression. Rats were additionally treated twice weekly with RAP-011 (2.5 mg/kg, s.c.) or vehicle (PBS) from week 5 to week 9 post SU5416, at which time treatment was withdrawn for the remaining 4 weeks. The following parameters were determined as a function of treatment: RVSP (FIG. 18B), TPRI (FIG. 18C), Fulton index (FIG. 18D), CI (FIG. 18E), PAAT (FIG. 18F), and TAPSE (FIG. 18G). Data are means±SEM (n=7-13 rats per group). Analysis by one-way ANOVA and Tukey post hoc test (*P<0.05, **P<0.01, ****P<0.0001).

FIG. 19 shows components of a kit comprising a lyophilized polypeptide and an injection device. A vial (1) holds lyophilized polypeptide, reconstituted sterile injectable solution, or sterile injectable solution. A prefilled syringe (2) containing a reconstitution solution used to reconstitute lyophilized polypeptide from (1) into a sterile injectable solution. A vial adapter (3) couples the vial (1) to the pre-filled syringe (2) via attachment to the vial at one end, and attachment to the pre-filled syringe at an opposite end. A syringe (4) and needle (5) are provided for administration of sterile injectable solution. Swab wipes (6) are provided for sterilization of individual kit components.

FIGS. 20A-C show the mean change from baseline to week 24 in echocardiography parameters measured during a placebo-controlled trial using sotatercept (an ActRIIA-hFc polypeptide) treatment in patients with pulmonary arterial hypertension. FIG. 20A shows the improvement in LS mean (SE) change from baseline to week 24 in right ventricular end-diastolic area (RVEDA). FIG. 20B shows the improvement in LS mean (SE) change from baseline to week 24 in right ventricular end-systolic area (RVESA). FIG. 20C shows the LS mean and p-value data from FIG. 20A and FIG. 20B in table format. Baseline data are mean (SD) and Change are LS Mean (SE) from the evaluable analysis set. All echocardiography data was obtained in 2D. Bar graphs represent mean±standard deviation. †EOP represents data obtained at the end of the placebo-controlled treatment period (24 weeks). ‡Standard error is represented in parentheses for all LS mean values. CI: confidence interval; EOP: end of placebo-controlled treatment period; LS: least squares; SOC: standard of care.

FIGS. 21A and 21B show the mean change from baseline to week 24 in pulmonary artery systolic pressure measured during a placebo-controlled trial using sotatercept (an ActRIIA-hFc polypeptide) treatment in patients with pulmonary arterial hypertension. FIG. 21A shows the improvement in pulmonary artery systolic pressure (PASP) in patients treated with 0.3 mg/kg sotatercept and standard of care (SOC) or 0.7 mg/kg of sotatercept and SOC. FIG. 21B shows the LS mean and p value data from FIG. 21A in table format. Baseline data are mean (SD) and Change are LS Mean (SE) from the evaluable analysis set. All echocardiography data was obtained in 2D. Bar graphs represent mean±standard deviation. †EOP represents data obtained at the end of the placebo-controlled treatment period (24 weeks). ‡Standard error is represented in parentheses for all LS mean values. CI: confidence interval; LS: least squares.

FIGS. 22A and 22B show the mean change from baseline to week 24 in right ventricle-pulmonary artery (RV-PA) coupling measured during a placebo-controlled trial using sotatercept (an ActRIIA-hFc polypeptide) treatment in patients with pulmonary arterial hypertension. FIG. 22A shows the improvement in RV-PA coupling in patients treated with 0.3 mg/kg sotatercept and standard of care (SOC) or 0.7 mg/kg sotatercept and SOC. FIG. 22B shows the LS mean a p value data from FIG. 22A in table format. Baseline data are mean (SD) and Change are LS Mean (SE) from the evaluable analysis set. All echocardiography data was obtained in 2D. Bar graphs represent mean±standard deviation, †EOP represents data obtained at the end of the placebo-controlled treatment period (24 weeks). ‡Standard error is represented in parentheses for all LS mean values. § Cut-off values for RV-PA coupling have not been validated in a large cohort. CI: confidence interval; LS: least squares; parentheses for all LS mean values; RV-PA: right ventricular-pulmonary artery.

FIGS. 23A-F show that treatment with an ActRIIA-mFc polypeptide prevents PH and reduces right ventricular hypertrophy in a mouse model of BMPR2 haploinsufficiency. The experimental strategy used to test preventive effects of ActRIIA-mFc in the mouse model of Bmpr2 haploinsufficiency is shown in FIG. 23A. Twenty-nine Bnmpr2^{+/R899X} mice were randomized into three groups: (i) seven mice were housed in normoxic conditions for 5 weeks, “Nx”; (ii) eleven mice were housed in hypoxic conditions and injected subcutaneously with vehicle control (phosphate buffered saline (PBS)), twice weekly for 5 weeks, “Hx Veh”; and (iii) eleven mice were housed in hypoxic conditions and injected subcutaneously with ActRIIA-mFc at a dose of 10 mg/kg twice weekly for 5 weeks, “Hx ActRIIA-mFc.” FIG. 23B shows the pulmonary artery acceleration time (PAAT) and FIG. 23C shows the right ventricular systolic pressure (RVSP). FIG. 23D shows the right ventricular free wall thickness (RVWT) and FIG. 23E shows the Fulton index, calculated as the ratio of right ventricular weight (RV) to weight of the combined left ventricle and septum (LV+S) (RV/(LV+S)). FIG. 23F shows the tricuspid annular plane systolic excursion (TAPSE). Data are means±SEM (n=7-11 per group). Analysis by one-way ANOVA and Tukey post hoc test. *P<0.05, ***P<0.001, ****P<0.0001.

FIG. 24A and FIG. 24B show that treatment with an ActRIIA-mFc polypeptide prevents perivascular inflammation by preventing macrophage infiltration in the lung. Twenty-nine Bmpr2^{+/R899X} mice were randomized into three groups: (i) seven mice were housed in normoxic conditions for 5 weeks, “Nx”; (ii) eleven mice were housed in hypoxic conditions and injected subcutaneously with vehicle control (phosphate buffered saline (PBS)), twice weekly for 5 weeks, “Hx Veh”; and (iii) eleven mice were housed in hypoxic conditions and injected subcutaneously with ActRIIA-mFc at a dose of 10 mg/kg twice weekly for 5 weeks, “Hx ActRIIA-mFc.” FIG. 24A shows a post-mortem analysis of macrophage infiltration in the lung by performing an immunohistochemical staining for macrophage marker F4/80. FIG. 24B shows a quantification of the percentage of F4/80-positive cells in the lung based on assessment of 40 high-magnification fields per animal. Data are means±SEM (n=7-11 per group). Analysis by one-way ANOVA and Tukey post hoc test. *P<0.05, ***P<0.001, ****P<0.0001.

### DETAILED DESCRIPTION

### 1. Overview

The present disclosure relates to compositions and methods of treating pulmonary arterial hypertension (e.g., functional class II or functional class III) comprising administering to a patient in need thereof an effective amount of an ActRII polypeptide as described herein. In certain embodiments, the present disclosure provides methods of treating or preventing pulmonary arterial hypertension in an individual in need thereof through administering to the individual a therapeutically effective amount of an ActRII polypeptide as described herein.

Pulmonary arterial hypertension [World Health Organization (WHO) Group 1 PH] is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary artery pressures. These high pressures make it difficult for the heart to pump blood through the lungs to be oxygenated. Patients with PAH suffer from extreme shortness of breath as the heart struggles to pump against these high pressures. Patients with PAH typically develop significant increases in PVR and sustained elevations in mPAP, which ultimately lead to right ventricular failure and death. Patients diagnosed with PAH have a poor prognosis and equally compromised quality of life, with a mean life expectancy of 2 to 5 years from the time of diagnosis if untreated.

PAH can be diagnosed based on a mean pulmonary artery pressure of above 25 mmHg (or above 20 mmHg under updated guidelines) at rest, with a normal pulmonary artery capillary wedge pressure. PAH can lead to shortness of breath, dizziness, fainting, and other symptoms, all of which are exacerbated by exertion. PAH can be a severe disease with a markedly decreased exercise tolerance and heart failure. Two major types of PAH include idiopathic PAH (e.g., PAH in which no predisposing factor is identified) and heritable PAH (e.g., PAH associated with a mutation in BMPR2, ALK1, ENG, SMAD9, CAV1, KCNK3, or EIF2AK4). In 70% of familial PAH cases, mutations are located in the BMPR2 gene. Risk factors for the development of PAH include family history of PAH, drug and toxin use (e.g., methamphetamine or cocaine use), infection (e.g., HIV infection or schistosomiasis), cirrhosis of the liver, congenital heart abnormalities, portal hypertension, pulmonary veno-occlusive disease, pulmonary capillary hemangiomatosis, or connective tissue/autoimmune disorders (e.g., scleroderma or lupus). PAH may be associated with long term responders to calcium channel blockers, overt features of venous/capillaries (PVOD/PCH) involvement, and persistent PH of the newborn syndrome.

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST (Basic Local Alignment Search Tool), BLAST-2, ALIGN, ALIGN-2, Clustal Omega, or Megalign (DNASTAR) software. Those skilled in the art can determine appropriate parameters for aligning sequences, including any algorithms needed to achieve maximal alignment over the full length of the sequences being compared. In some embodiments, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. Other algorithms for determining sequence identity or homology include: Clustal Omega (http://www.ebi.ac.uk/Tools/msa/clustalo/), LALIGN (http://www.ebi.ac.uk/Tools/psa/lalign/ and http://www.ebi.ac.uk/Tools/psa/lalign/nucleotide.html), FASTA (http://www.ebi.ac.uk/Tools/sss/fasta/), SIM (http://web.expasy.org/sim/), and EMBOSS Needle (https://www.ebi.ac.uk/Tools/psa/emboss_needle/). In a preferred embodiment, the algorithm used for determining sequence identity is Clustal Omega.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is ±10%. Alternatively, and particularly in biological systems, the terms “about” and “approximately” may mean values that are within an order of magnitude, preferably ≤5-fold and more preferably −2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. ActRII Polypeptides

In certain aspects, the disclosure relates to ActRII polypeptides and uses thereof (e.g., of treating, preventing, or reducing the progression rate and/or severity of pulmonary arterial hypertension or one or more complications of pulmonary arterial hypertension). As used herein, the term “ActRII” refers to the family of type II activin receptors. This family includes activin receptor type IIA (ActRIIA) and activin receptor type IIB (ActRIIB).

In certain embodiments, the present disclosure relates to ActRII polypeptides having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence as set forth in anyone of SEQ ID NOs: 1, 2, 3, 23, 27, 30, and 41. In other embodiments, the present disclosure relates to ActRII polypeptides having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence as set forth in SEQ ID NO: 31. As used herein, the term “ActRII” refers to a family of activin receptor type IIA (ActRIIA) proteins, a family of activin receptor type IIB (ActRIIB) proteins, or combinations and/or variants thereof. The ActRII polypeptides can be derived from any species and include variants derived from such ActRII proteins by mutagenesis or other modification. Reference to ActRII herein is understood to be a reference to any one of the currently identified forms. Members of the ActRII family are generally transmembrane proteins, composed of a ligand-binding extracellular domain comprising a cysteine-rich region, a transmembrane domain, and a cytoplasmic domain with predicted serine/threonine kinase activity.

The term ActRII polypeptide includes polypeptides comprising any naturally occurring polypeptide of an ActRII family member as well as any variants thereof (including mutants, fragments, fusions, and peptidomimetic forms) that retain a useful activity. Examples of such variant ActRII polypeptides are provided throughout the present disclosure as well as in International Patent Application Publication Nos. WO 2006/012627, WO 2007/062188, WO 2008/097541, WO 2010/151426, and WO 2011/020045, which are incorporated herein by reference in their entirety. Numbering of amino acids for all ActRII-related polypeptides described herein is based on the numbering of the human ActRII precursor protein sequence provided below (SEQ ID NO: 1), unless specifically designated otherwise.

The canonical human ActRII precursor protein sequence is as follows:

| (SEQ ID NO: 1) | |
|---|---|
| 1 | MGAAAKLAFA VELISCSSGA ILGRSETQEC LFFNANWEKD RTNQTGVEPC |
| 51 | YGDKDKRRHC FATWKNISGS IEIVKQGCWL DDINCYDRTD CVEKKDSPEV |
| 101 | YFCCCEGNMC NEKFSYFPEM EVTQPTSNPV TPKPPYYNIL LYSLVPLMLI |
| 151 | AGIVICAFWV YRHHKMAYPP VLVPTQDPGP PPPSPLLGLK PLQLLEVKAR |
| 201 | GRFGCVWKAQ LLNEYVAVKI FPIQDKQSWQ NEYEVYSLPG MKHENILQFI |
| 251 | GAEKRGTSVD VDLWLITAFH EKGSLSDFLK ANVVSWNELC HIAETMARGL |
| 301 | AYLHEDIPGL KDGHKPAISH RDIKSKNVLL KNNLTACIAD FGLALKFEAG |
| 351 | KSAGDTHGQV GTRRYMAPEV LEGAINFQRD AFLRIDMYAM GLVLWELASR |
| 401 | CTAADGPVDE YMLPFEEEIG QHPSLEDMQE VVVHKKKRPV LRDYWQKHAG |
| 451 | MAMLCETIEE CWDHDAEARL SAGCVGERIT QMQRLTNIIT TEDIVTVVTM |
| 501 | VTNVDFPPKE SSL |

The signal peptide is indicated by a single underline; the extracellular domain is indicated in bold font; and the potential, endogenous N-linked glycosylation sites are indicated by a d̅o̅u̅b̅l̅e̅ u̅n̅d̅e̅r̅l̅i̅n̅e̅.

A processed (mature) extracellular human ActRII polypeptide sequence is as follows:

(SEQ ID NO: 2)
ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPP

The C-terminal “tail” of the extracellular domain is indicated by single underline. The sequence with the “tail” deleted (a Δ15 sequence) is as follows:

(SEQ ID NO: 3)
ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EM

The nucleic acid sequence encoding human ActRII precursor protein is shown below (SEQ ID NO: 4), as follows nucleotides 159-1700 of Genbank Reference Sequence NM_001616.4. The signal sequence is underlined.

| (SEQ ID NO: 4) | |
|---|---|
| 1 | ATGGGAGCTG CTGCAAAGTT GGCGTTTGCC GTCTTTCTTA TCTCCTGTTC |
| 51 | TTCAGGTGCT ATACTTGGTA GATCAGAAAC TCAGGAGTGT CTTTTCTTTA |
| 101 | ATGCTAATTG GGAAAAAGAC AGAACCAATC AAACTGGTGT TGAACCGTGT |
| 151 | TATGGTGACA AAGATAAACG GCGGCATTGT TTTGCTACCT GGAAGAATAT |
| 201 | TTCTGGTTCC ATTGAAATAG TGAAACAAGG TTGTTGGCTG GATGATATCA |
| 251 | ACTGCTATGA CAGGACTGAT TGTGTAGAAA AAAAAGACAG CCCTGAAGTA |
| 301 | TATTTTTGTT GCTGTGAGGG CAATATGTGT AATGAAAAGT TTTCTTATTT |
| 351 | TCCGGAGATG GAAGTCACAC AGCCCACTTC AAATCCAGTT ACACCTAAGC |
| 401 | CACCCTATTA CAACATCCTG CTCTATTCCT TGGTGCCACT TATGTTAATT |
| 451 | GCGGGGATTG TCATTTGTGC ATTTTGGGTG TACAGGCATC ACAAGATGGC |
| 501 | CTACCCTCCT GTACTTGTTC CAACTCAAGA CCCAGGACCA CCCCCACCTT |
| 551 | CTCCATTACT AGGTTTGAAA CCACTGCAGT TATTAGAAGT GAAAGCAAGG |
| 601 | GGAAGATTTG GTTGTGTCTG GAAAGCCCAG TTGCTTAACG AATATGTGGC |
| 651 | TGTCAAAATA TTTCCAATAC AGGACAAACA GTCATGGCAA AATGAATACG |
| 701 | AAGTCTACAG TTTGCCTGGA ATGAAGCATG AGAACATATT ACAGTTCATT |
| 751 | GGTGCAGAAA AACGAGGCAC CAGTGTTGAT GTGGATCTTT GGCTGATCAC |
| 801 | AGCATTTCAT GAAAAGGGTT CACTATCAGA CTTTCTTAAG GCTAATGTGG |
| 851 | TCTCTTGGAA TGAACTGTGT CATATTGCAG AAACCATGGC TAGAGGATTG |
| 901 | GCATATTTAC ATGAGGATAT ACCTGGCCTA AAAGATGGCC ACAAACCTGC |
| 951 | CATATCTCAC AGGGACATCA AAAGTAAAAA TGTGCTGTTG AAAAACAACC |
| 1001 | TGACAGCTTG CATTGCTGAC TTTGGGTTGG CCTTAAAATT TGAGGCTGGC |
| 1051 | AAGTCTGCAG GCGATACCCA TGGACAGGTT GGTACCCGGA GGTACATGGC |
| 1101 | TCCAGAGGTA TTAGAGGGTG CTATAAACTT CCAAAGGGAT GCATTTTTGA |
| 1151 | GGATAGATAT GTATGCCATG GGATTAGTCC TATGGGAACT GGCTTCTCGC |
| 1201 | TGTACTGCTG CAGATGGACC TGTAGATGAA TACATGTTGC CATTTGAGGA |
| 1251 | GGAAATTGGC CAGCATCCAT CTCTTGAAGA CATGCAGGAA GTTGTTGTGC |
| 1301 | ATAAAAAAAA GAGGCCTGTT TTAAGAGATT ATTGGCAGAA ACATGCTGGA |
| 1351 | ATGGCAATGC TCTGTGAAAC CATTGAAGAA TGTTGGGATC ACGACGCAGA |
| 1401 | AGCCAGGTTA TCAGCTGGAT GTGTAGGTGA AAGAATTACC CAGATGCAGA |
| 1451 | GACTAACAAA TATTATTACC ACAGAGGACA TTGTAACAGT GGTCACAATG |
| 1501 | GTGACAAATG TTGACTTTCC TCCCAAAGAA TCTAGTCTA |

The nucleic acid sequence encoding processed soluble (extracellular) human ActRII polypeptide is as follows:

| (SEQ ID NO: 5) | |
|---|---|
| 1 | ATACTTGGTA GATCAGAAAC TCAGGAGTGT CTTTTCTTTA ATGCTAATTG |
| 51 | GGAAAAAGAC AGAACCAATC AAACTGGTGT TGAACCGTGT TATGGTGACA |
| 101 | AAGATAAACG GCGGCATTGT TTTGCTACCT GGAAGAATAT TTCTGGTTCC |
| 151 | ATTGAAATAG TGAAACAAGG TTGTTGGCTG GATGATATCA ACTGCTATGA |
| 201 | CAGGACTGAT TGTGTAGAAA AAAAAGACAG CCCTGAAGTA TATTTTTGTT |
| 251 | GCTGTGAGGG CAATATGTGT AATGAAAAGT TTTCTTATTT TCCGGAGATG |
| 301 | GAAGTCACAC AGCCCACTTC AAATCCAGTT ACACCTAAGC CACCC |

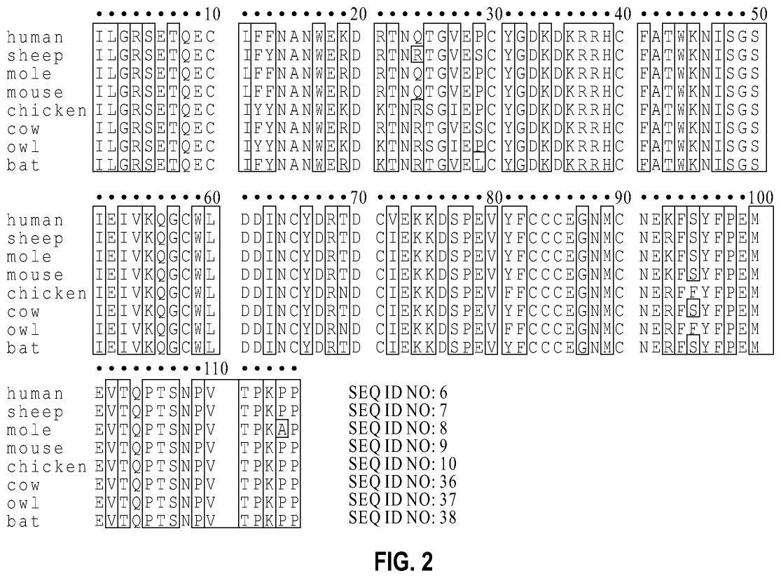

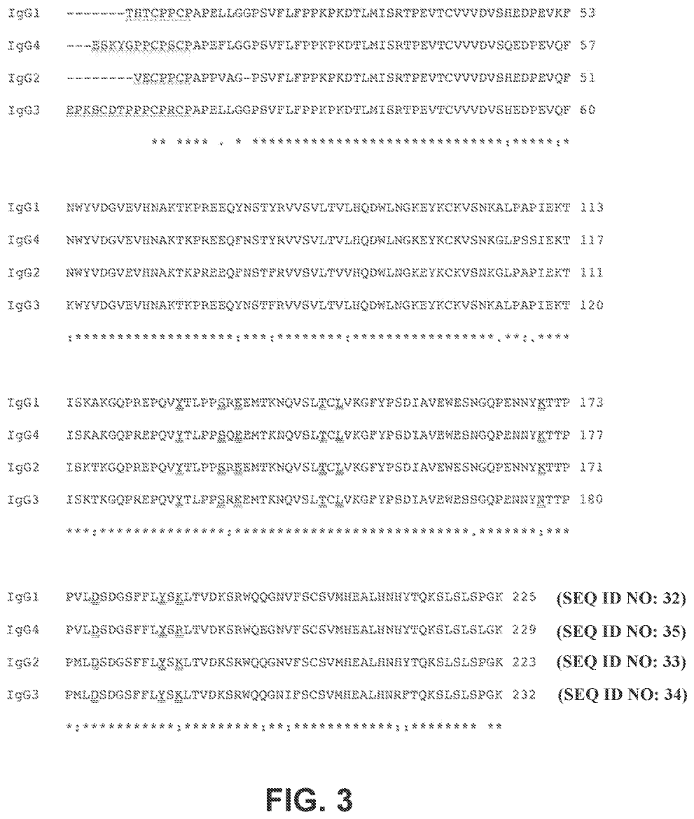

ActRII is well-conserved among vertebrates, with large stretches of the extracellular domain completely conserved. For example, FIG. 2 depicts a multi-sequence alignment of a human ActRIIA extracellular domain compared to various ActRIIA orthologs. Many of the ligands that bind to ActRIIA are also highly conserved. Accordingly, from these alignments, it is possible to predict key amino acid positions within the ligand-binding domain that are important for normal ActRII-ligand binding activities as well as to predict amino acid positions that are likely to be tolerant to substitution without significantly altering normal ActRII-ligand binding activities. Therefore, an active, human ActRII variant polypeptide useful in accordance with the presently disclosed methods may include one or more amino acids at corresponding positions from the sequence of another vertebrate ActRII, or may include a residue that is similar to that in the human or other vertebrate sequences.

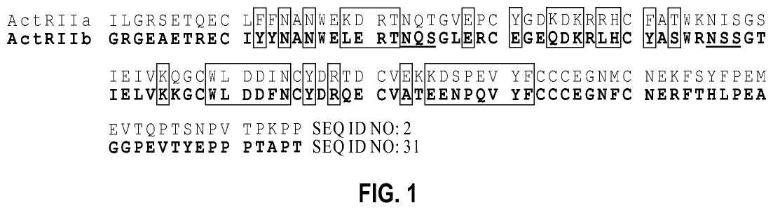

An alignment of the amino acid sequences of human ActRIIA extracellular domain and human ActRIIB extracellular domain are illustrated in FIG. 1. This alignment indicates amino acid residues within both receptors that are believed to directly contact ActRII ligands. For example, the composite ActRII structures indicated that the ActRIIA-ligand binding pocket is defined, in part, by residues F31, N33, N35, K38 through T41, E47, Y50, K53 through K55, R57, H58, F60, T62, K74, W78 through N83, Y85, R87, E92, and K94 through F101. At these positions, it is expected that conservative mutations will be tolerated.

Without meaning to be limiting, the following examples illustrate this approach to defining an active ActRII variant. As illustrated in FIG. 2, F13 in the human extracellular domain is Y in Ovis aries (SEQ ID NO: 7), Gallus gallus (SEQ ID NO: 10), Bos Taurus (SEQ ID NO: 36), Tyto alba (SEQ ID NO: 37), and Myotis davidii (SEQ ID NO: 38) ActRIIA, indicating that aromatic residues are tolerated at this position, including F, W, and Y. Q24 in the human extracellular domain is R in Bos Taurus ActRIIA, indicating that charged residues will be tolerated at this position, including D, R, K, H, and E. S95 in the human extracellular domain is F in Gallus gallus and Tyto alba ActRIIA, indicating that this site may be tolerant of a wide variety of changes, including polar residues, such as E, D, K, R, H, S, T, P, G, Y, and probably hydrophobic residue such as L, 1, or F. E52 in the human extracellular domain is D in Ovis aries ActRIIA, indicating that acidic residues are tolerated at this position, including D and E. P29 in the human extracellular domain is relatively poorly conserved, appearing as S in Ovis aries ActRIIA and L in Myotis davidii ActRIIA, thus essentially any amino acid should be tolerated at this position.

Moreover, as discussed above, ActRII proteins have been characterized in the art in terms of structural/functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663]. For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. In addition to the teachings herein, these references provide amply guidance for how to generate ActRII variants that retain one or more desired activities (e.g., ligand-binding activity).

For example, a defining structural motif known as a three-finger toxin fold is important for ligand binding by type I and type TT receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRII, as demarcated by the outermost of these conserved cysteines, corresponds to positions 30-110 of SEQ ID NO: 1 (ActRII precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24,or 25 residues at the C-terminus without necessarily altering ligand binding. Exemplary ActRII extracellular domains truncations include SEQ ID NOs: 2 and 3.

Accordingly, a general formula for an active portion (e.g., ligand binding) of ActRII is a polypeptide that comprises, consists essentially of, or consists of amino acids 30-110 of SEQ ID NO: 1. Therefore ActRII polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRII beginning at a residue corresponding to any one of amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 1. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 1, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 1. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 1. Thus, in some embodiments, an ActRII polypeptide may comprise, consists essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1. Optionally, ActRII polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1, and comprising no more than 1, 2, 5, 10 or 15 conservative amino acid changes in the ligand-binding pocket. In some embodiments, the ActRII polypeptide is part of a homodimer protein complex.

In certain embodiments, the disclosure relates to an ActRII polypeptide (e.g., ActRIIA polypeptides, ActRIIB polypeptides, or combinations thereof), which includes fragments, functional variants, and modified forms thereof as well as uses thereof (e.g., treating, preventing, or reducing the pulmonary arterial hypertension). Preferably, ActRII polypeptides are soluble (e.g., an extracellular domain of ActRII). In some embodiments, ActRII polypeptides inhibit (e.g., Smad signaling) of one or more GDF/BMP ligands [e.g., GDF11, GDF8, activin A, activin B, GDF3, BMP4, BMP6, BMP10, and/or BMP15]. In some embodiments, ActRII polypeptides bind to one or more GDF/BMP ligands [e.g., GDF11, GDF8, activin A, activin B, GDF3, BMP4, BMP6, BMP10, and/or BMP15]. In some embodiments, ActRII polypeptide of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRII beginning at a residue corresponding to amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134 or 135) of SEQ ID NO: 1. In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 1. In certain embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 1. In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 23, 27, 30, and 41.

In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 23. In some alternative embodiments, the ActRII polypeptide (e.g., SEQ ID NO: 23) may lack the C-terminal lysine. In some embodiments, the ActRII polypeptide lacking the C-terminal lysine is SEQ ID NO: 41. In some embodiments, the ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 41. In some embodiments, a patient is administered an ActRII polypeptide comprising, consisting, or consisting essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 23. In some embodiments, a patient is administered an ActRII polypeptide comprising, consisting, or consisting essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 41. In some embodiments, a patient is administered a combination of SEQ ID NO: 23 and SEQ ID NO: 41.

In certain aspects, the present disclosure relates to ActRII polypeptides (e.g., ActRIIA polypeptides, ActRIIB polypeptides, or combinations thereof). In some embodiments, ActRII traps of the present disclosure are variant ActRII polypeptides (e.g., ActRIIA polypeptides, ActRIIB polypeptides, or combinations thereof) that comprise one or more mutations (e.g., amino acid additions, deletions, substitutions, and combinations thereof) in the extracellular domain (also referred to as the ligand-binding domain) of an ActRII polypeptide (e.g., a “wild-type” or unmodified ActRII polypeptide) such that the variant ActRII polypeptide has one or more altered ligand-binding activities than the corresponding wild-type ActRII polypeptide. In preferred embodiments, variant ActRII polypeptides of the present disclosure retain at least one similar activity as a corresponding wild-type ActRII polypeptide. For example, preferable ActRII polypeptides bind to and inhibit (e.g. antagonize) the function of GDF11 and/or GDF8. In some embodiments, ActRII polypeptides of the present disclosure further bind to and inhibit one or more of ligand of the GDF/BMP [e.g., GDF11, GDF8, activin A, activin B, GDF3, BMP4, BMP6, BMP10, and/or BMP15]. Accordingly, the present disclosure provides ActRII polypeptides that have an altered binding specificity for one or more ActRII ligands.

To illustrate, one or more mutations may be selected that increase the selectivity of the altered ligand-binding domain for GDF11 and/or GDF8 over one or more ActRII-binding ligands such as activins (activin A or activin B), particularly activin A. Optionally, the altered ligand-binding domain has a ratio of K_{d} for activin binding to K_{d} for GDF11 and/or GDF8 binding that is at least 2-, 5-, 10-, 20-, 50-, 100- or even 1000-fold greater relative to the ratio for the wild-type ligand-binding domain. Optionally, the altered ligand-binding domain has a ratio of IC₅₀ for inhibiting activin to IC₅₀ for inhibiting GDF11 and/or GDF8 that is at least 2-, 5-, 10-, 20-, 50-, 100- or even 1000-fold greater relative to the wild-type ligand-binding domain. Optionally, the altered ligand-binding domain inhibits GDF11 and/or GDF8 with an IC₅₀ at least 2-, 5-, 10-, 20-, 50-, 100- or even 1000-times less than the IC₅₀ for inhibiting activin.

In certain embodiments, the present disclosure contemplates specific mutations of an ActRII polypeptide (e.g., ActRIIA polypeptides, ActRIIB polypeptides, or combinations thereof) so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, polypeptides of the present disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of an ActRII polypeptide (e.g., ActRIIA polypeptides. ActRIIB polypeptides, or combinations thereof) as well as truncation mutants. Pools of combinatorial mutants are especially useful for identifying functionally active (e.g., GDF/BMP ligand binding) ActRII sequences. The purpose of screening such combinatorial libraries may be to generate, for example, polypeptides variants, which have altered properties, such as altered pharmacokinetic or altered ligand binding. A variety of screening assays are provided below, and such assays may be used to evaluate variants. For example, ActRII variants may be screened for ability to bind to one or more GDF/BMP ligands [e.g., GDF11, GDF8, activin A, activin B, GDF3, BMP4, BMP6, BMP10, and/or BMP15], to prevent binding of a GDF/BMP ligand to an ActRII polypeptide, as well as heteromultimers thereof, and/or to interfere with signaling caused by an GDF/BMP ligand.

The activity of ActRII polypeptides (e.g., ActRIIA polypeptides, ActRIIB polypeptides, or combinations thereof) or variants thereof may also be tested in a cell-based or in vivo assay. For example, the effect of an ActRII polypeptide on the expression of genes involved in pulmonary arterial hypertension pathogenesis may be assessed. This may, as needed, be performed in the presence of one or more recombinant ligand proteins [e.g., GDF11, GDF8, activin A, activin B, GDF3, BMP4, BMP6, BMP10, and/or BMP15], and cells may be transfected so as to produce an ActRII polypeptide, and optionally, an GDF/BMP ligand. Likewise, an ActRII polypeptide may be administered to a mouse or other animal and effects on pulmonary arterial hypertension pathogenesis may be assessed using art-recognized methods. Similarly, the activity of an ActRII polypeptide or variant thereof may be tested in blood cell precursor cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference ActRII polypeptide (e.g., ActRIIA polypeptides, ActRIIB polypeptides, or combinations thereof). Such variants, when expressed from recombinant DNA constructs, can be used in gene therapy protocols. Likewise, mutagenesis can give rise to variants which have intracellular half-lives dramatically different than the corresponding unmodified ActRII polypeptide. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter the half-life of the ActRII polypeptide.

A combinatorial library may be produced by way of a degenerate library of genes encoding a library of polypeptides which each include at least a portion of potential ActRII polypeptide sequences. For instance, a mixture of synthetic oligonucleotides can be enzymatically ligated into gene sequences such that the degenerate set of potential ActRII encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art [Narang, S A (1983) Tetrahedron 39:3; Itakura et al. (1981) Recombinant DNA, Proc. 3rd Cleveland Sympos. Macromolecules, ed. AG Walton, Amsterdam: Elsevier pp273-289; Itakura et al. (1984) Annu. Rev. Biochem. 53:323; Itakura et al. (1984) Science 198:1056; and Ike et al. (1983) Nucleic Acid Res. 11:477]. Such techniques have been employed in the directed evolution of other proteins [Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos. 5,223,409, 5,198,346, and 5,096,815].

Alternatively, other forms of mutagenesis can be utilized to generate a combinatorial library. For example, ActRII polypeptides of the disclosure (e.g., ActRIIA polypeptides, ActRIIB polypeptides, or combinations thereof) can be generated and isolated from a library by screening using, for example, alanine scanning mutagenesis [Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [Gustin et al. (1993) Virology 193:653-660; and Brown et al. (1992) Mol. Cell Biol. 12:2644-2652; McKnight et al. (1982) Science 232:316], by saturation mutagenesis [Meyers et al., (1986) Science 232:613]; by PCR mutagenesis [Leung et al. (1989) Method Cell Mol Biol 1:11-19]; or by random mutagenesis, including chemical mutagenesis [Miller et al. (1992) A Short Course in Bacterial Genetics, CSHL Press, Cold Spring Harbor, NY; and Greener et al. (1994) Strategies in Mol Biol 7:32-34]. Linker scanning mutagenesis, particularly in a combinatorial setting, is an attractive method for identifying truncated (bioactive) forms of ActRII polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of ActRII polypeptides (e.g., ActRIIA polypeptides, ActRIIB polypeptides, or combinations thereof). The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include ligand [e.g., GDF11, GDF8, activin A, activin B, GDF3, BMP4, BMP6, BMP10, and/or BMP15] binding assays and/or ligand-mediated cell signaling assays.

As will be recognized by one of skill in the art, most of the described mutations, variants or modifications described herein may be made at the nucleic acid level or, in some cases, by post-translational modification or chemical synthesis. Such techniques are well known in the art and some of which are described herein. In part, the present disclosure identifies functionally active portions (fragments) and variants of ActRII polypeptides (e.g., ActRIIA polypeptides. ActRIIB polypeptides, or combinations thereof) that can be used as guidance for generating and using other variant ActRII polypeptides within the scope of the disclosure provided herein.

In certain embodiments, functionally active fragments of ActRII polypeptides of the present disclosure can be obtained by screening polypeptides recombinantly produced from the corresponding fragment of the nucleic acid encoding an ActRII polypeptide. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (inhibitors) of ActRII receptors and/or one or more ligands [e.g., GDF11, GDF8, activin A, activin B, GDF3, BMP4, BMP6, BMP10, and/or BMP15].

In certain embodiments, ActRII polypeptides of the present disclosure (e.g., ActRIIA polypeptides, ActRIIB polypeptides, or combinations thereof) may further comprise post-translational modifications in addition to any that are naturally present in the ActRII polypeptide. Such modifications include, but are not limited to, acetylation, carboxylation, glycosylation, phosphorylation, lipidation, and acylation. As a result, the ActRII polypeptide may contain non-amino acid elements, such as polyethylene glycols, lipids, polysaccharide or monosaccharide, and phosphates. Effects of such non-amino acid elements on the functionality of a ligand trap polypeptide may be tested as described herein for other ActRII variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, W138, NIH-3T3 or HEK293) have specific cellular machinery and characteristic mechanisms for such post-translational activities and may be chosen to ensure the correct modification and processing of the ActRII polypeptides.

In certain aspects, ActRII polypeptides of the present disclosure (e.g., ActRIIA polypeptides, ActRIIB polypeptides, or combinations thereof) include fusion proteins having at least a portion (domain) of an ActRII polypeptide and one or more heterologous portions (domains). Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the ActRII polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation. Other types of fusion domains that may be selected include multimerizing (e.g., dimerizing, tetramerizing) domains and functional domains (that confer an additional biological function) including, for example constant domains from immunoglobulins (e.g., Fc domains).

In certain aspects, ActRII polypeptides of the present disclosure (e.g., ActRIIA polypeptides, ActRIIB polypeptides, or combinations thereof) contain one or more modifications that are capable of “stabilizing” the polypeptides. By “stabilizing” is meant anything that increases the in vitro half-life, serum half-life, regardless of whether this is because of decreased destruction, decreased clearance by the kidney, or other pharmacokinetic effect of the agent. For example, such modifications enhance the shelf-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising an ActRII polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes nonproteinaceous modifications such as a carbohydrate moiety, or nonproteinaceous moiety, such as polyethylene glycol. In certain preferred embodiments, an ActRII polypeptide is fused with a heterologous domain that stabilizes the polypeptide (a “stabilizer” domain), preferably a heterologous domain that increases stability of the polypeptide in vivo. Fusions with a constant domain of an immunoglobulin (e.g., a Fc domain) are known to confer desirable pharmacokinetic properties on a wide range of proteins. Likewise, fusions to human serum albumin can confer desirable properties.

An example of a native amino acid sequence that may be used for the Fc portion of human 1gG1 (G1Fc) is shown below (SEQ ID NO: 11). Dotted underline indicates the hinge region, and solid underline indicates positions with naturally occurring variants. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 11. Naturally occurring variants in G1Fc would include E134D and M136L according to the numbering system used in SEQ ID NO: 11 (see Uniprot P01857).

| (SEQ ID NO: 11) | |
|---|---|
| 1 | 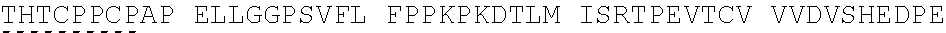 |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLIV DKSRWQQGNV |
| 201 | FSCSVMHEAL HNHYTQKSLS LSPGK |

Optionally, the IgG1 Fc domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcγ receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain.

An example of a native amino acid sequence that may be used for the Fc portion of human IgG2 (G2Fc) is shown below (SEQ ID NO: 12). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 12.

| (SEQ ID NO: 12) | |
|---|---|
| 1 | 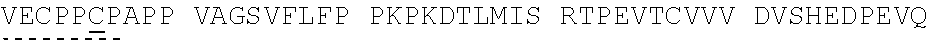 |
| 51 | FNWYVDGVEV HNAKTKPREE QFNSTFRVVS VLTVVHQDWL NGKEYKCKVS |
| 101 | NKGLPAPIEK TISKIKGQPR EPQVYTLPPS REEMTKNQVS LTCLVKGFYP |
| 151 | 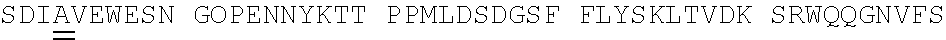 |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 13) contains a short hinge region consisting of a single 15-residue segment, whereas the second G3Fc sequence (SEQ ID NO: 14) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NOs: 13 and 14.

| (SEQ ID NO: 13) | |
|---|---|
| 1 | 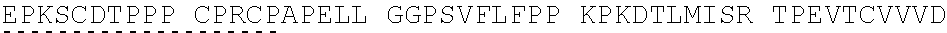 |
| 51 | VSHEDPEVQF KWYVDGVEVH NAKTKPREEQ YNSTFRVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP |
| (SEQ ID NO: 14) | |
| 1 | 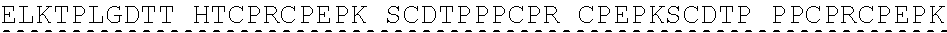 |
| 51 | 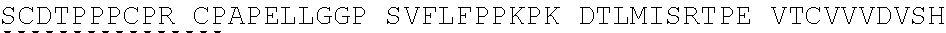 |
| 101 | EDPEVQFKWY VDGVEVHNAK TKPREEQYNS TFRVVSVLIV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 13, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human 1gG4 (G4Fc) is shown below (SEQ ID NO: 15). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 15.

| (SEQ ID NO: 15) | |
|---|---|
| 1 | 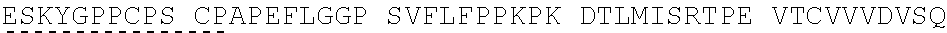 |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

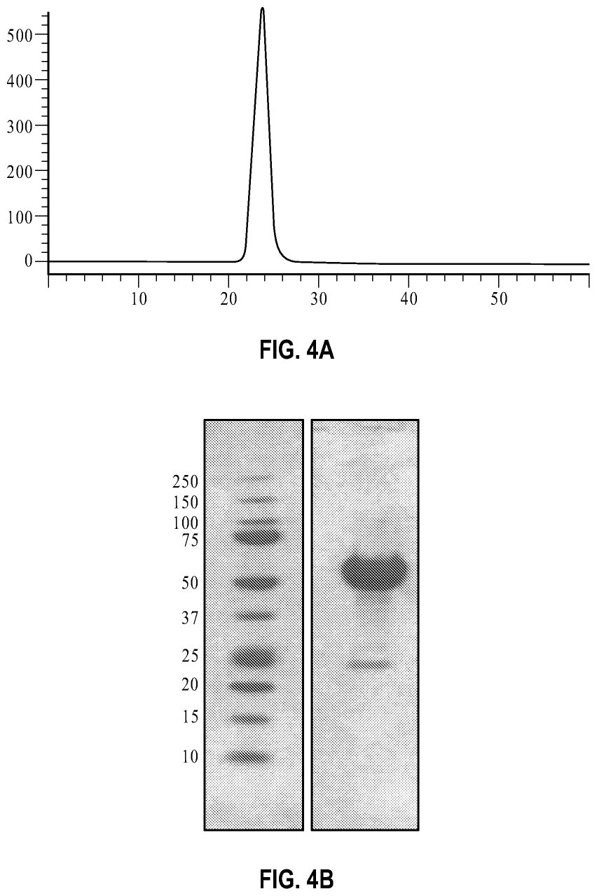

A variety of engineered mutations in the Fc domain are presented herein with respect to the G1Fc sequence (SEQ ID NO: 11), and analogous mutations in G2Fc, G3Fc, and G4Fc can be derived from their alignment with G1Fc in FIG. 4. Due to unequal hinge lengths, analogous Fc positions based on isotype alignment (FIG. 4) possess different amino acid numbers in SEQ ID NOs: 11, 12, 13, 14, and 15. It can also be appreciated that a given amino acid position in an immunoglobulin sequence consisting of hinge, C_{H}2, and C_{H}3 regions (e.g., SEQ ID NOs: 11, 12, 13, 14, and 15) will be identified by a different number than the same position when numbering encompasses the entire IgG1 heavy-chain constant domain (consisting of the C_{H}1, hinge, C_{H2}, and C_{H3} regions) as in the Uniprot database. For example, correspondence between selected C_{H}3 positions in a human G1Fc sequence (SEQ ID NO: 11), the human IgG1 heavy chain constant domain (Uniprot P01857), and the human IgG1 heavy chain is as follows.

Correspondence of C_{H}3 Positions in Different Numbering Systems

| | IgG1 heavy chain | |
|---|---|---|
| G1Fc | constant domain | IgG1 heavy chain |
| (Numbering begins at first | (Numbering begins | (EU numbering scheme |
| threonine in hinge region) | at C_{H}1) | of Kabat et al., 1991*) |
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

*Kabat et al. (eds) 1991; pp. 688-696 in Sequences of Proteins of Immunological Interest, 5^{th} ed., Vol. 1, NIH, Bethesda, MD.

Various methods are known in the art that increase desired pairing of Fc-containing fusion polypeptide chains in a single cell line to produce a preferred asymmetric fusion protein at acceptable yields [Klein et al (2012) mAbs 4:653-663; and Spiess et al (2015) Molecular Immunology 67(2A): 95-106]. Methods to obtain desired pairing of Fe-containing chains include, but are not limited to, charge-based pairing (electrostatic steering), “knobs-into-holes” steric pairing, SEEDbody pairing, and leucine zipper-based pairing [Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605].

It is understood that different elements of the fusion proteins (e.g., immunoglobulin Fe fusion proteins) may be arranged in any manner that is consistent with desired functionality. For example, an ActRII polypeptide domain may be placed C-terminal to a heterologous domain, or alternatively, a heterologous domain may be placed C-terminal to an ActRII polypeptide domain. The ActRII polypeptide domain and the heterologous domain need not be adjacent in a fusion protein, and additional domains or amino acid sequences may be included C- or N-terminal to either domain or between the domains.

For example, an ActRII receptor fusion protein may comprise an amino acid sequence as set forth in the formula A-B-C. The B portion corresponds to an ActRII polypeptide domain (e.g., ActRIIA polypeptides, ActRIIB polypeptides, or combinations thereof). The A and C portions may be independently zero, one, or more than one amino acid, and both the A and C portions when present are heterologous to B. The A and/or C portions may be attached to the B portion via a linker sequence. A linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 16), GGGG (SEQ ID NO: 17), TGGGG (SEQ ID NO: 18), SGGGG (SEQ ID NO: 19), TGGG (SEQ ID NO: 20), SGGG (SEQ ID NO: 21), or GGGGS (SEQ ID NO: 22) singlets, or repeats. In certain embodiments, an ActRII fusion protein comprises an amino acid sequence as set forth in the formula A-B-C, wherein A is a leader (signal) sequence, B consists of an ActRII polypeptide domain, and C is a polypeptide portion that enhances one or more of in vivo stability, in vivo half-life, uptake/administration, tissue localization or distribution, formation of protein complexes, and/or purification. In certain embodiments, an ActRII fusion protein comprises an amino acid sequence as set forth in the formula A-B-C, wherein A is a TPA leader sequence, B consists of an ActRII receptor polypeptide domain, and C is an immunoglobulin Fc domain. Preferred fusion proteins comprise the amino acid sequence set forth in any one of SEQ ID NOs: 23, 27, 30, and 41.

In preferred embodiments, ActRII polypeptides to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of purity are well known in the art [see, e.g., Flatman et al., (2007) J. Chromatogr. B 848:79-87]. In some embodiments, ActRII polypeptides to be used in accordance with the methods described herein are recombinant polypeptides.

ActRII polypeptides of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the polypeptides of the disclosure, including fragments or variants thereof, may be recombinantly produced using various expression systems [e.g., E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length ActRII polypeptides by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites. Alternatively, such polypeptides may be produced from recombinantly generated full-length ActRII polypeptides using chemical cleavage (e.g., cyanogen bromide, hydroxylamine, etc.).

### 3. Nucleic Acids Encoding ActRII Polypeptides

In certain embodiments, the present disclosure provides isolated and/or recombinant nucleic acids encoding ActRII polypeptides (e.g., ActRIIA polypeptides, ActRIIB polypeptides, or combinations thereof) including fragments, functional variants, and fusion proteins thereof.

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding ActRII polypeptides of the disclosure are understood to include nucleic acids that are variants of any one of SEQ ID NOs: 4, 5, or 28. Variant nucleotide sequences include sequences that differ by one or more nucleotide substitutions, additions, or deletions including allelic variants, and therefore, will include coding sequence that differ from the nucleotide sequence designated in any one of SEQ ID NOs: 4, 5, or 28.

In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to any one of SEQ ID NOs: 4, 5, or 28. One of ordinary skill in the art will appreciate that nucleic acid sequences that are at least 70%, 75%, 80%, 85%, 90%, 91%, 92%, 93%, 94% 95%, 96%, 97%, 98%, 99%, or 100% identical to the sequences complementary to SEQ ID NOs: 4, 5, or 28, and variants thereof, are also within the scope of the present disclosure. In further embodiments, the nucleic acid sequences of the disclosure can be isolated, recombinant, and/or fused with a heterologous nucleotide sequence, or in a DNA library.

In other embodiments, nucleic acids of the present disclosure also include nucleotide sequences that hybridize under highly stringent conditions to the nucleotide sequence designated in SEQ ID NOs: 4, 5, or 28, complement sequences of SEQ ID NOs: 4, 5, or 28, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 4, 5, or 28 to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art and can be used in a variety of host cells. Typically, one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and can vary with the host cell used.

In certain aspects, the subject nucleic acid disclosed herein is provided in an expression vector comprising a nucleotide sequence encoding an ActRII polypeptide (e.g., ActRIIA polypeptides, ActRIIB polypeptides, or combinations thereof) operably linked to at least one regulatory sequence. Regulatory sequences are art-recognized and are selected to direct expression of the ActRII polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding an ActRII polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ActRII polypeptide include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III).

In a preferred embodiment, a vector will be designed for production of the subject ActRII polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the subject ActRII polypeptides in cells propagated in culture, e.g., to produce proteins, including fusion proteins or variant proteins, for purification.

This disclosure also pertains to a host cell transfected with a recombinant gene including a coding sequence for one or more of the subject ActRII polypeptides. The host cell may be any prokaryotic or eukaryotic cell. For example, an ActRII polypeptide of the disclosure may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject ActRII polypeptides. For example, a host cell transfected with an expression vector encoding an ActRII polypeptide can be cultured under appropriate conditions to allow expression of the ActRII polypeptide to occur. The polypeptide may be secreted and isolated from a mixture of cells and medium containing the polypeptide. Alternatively, the ActRII polypeptide may be retained cytoplasmically or in a membrane fraction and the cells harvested, lysed and the protein isolated. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the ActRII polypeptides, and affinity purification with an agent that binds to a domain fused to the ActRII polypeptide (e.g., a protein A column may be used to purify an ActRII-Fc fusion proteins). In some embodiments, the ActRII polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. An ActRII protein may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant ActRII polypeptide, can allow purification of the expressed fusion protein by affinity chromatography using a Ni²⁺ metal resin. The purification leader sequence can then be subsequently removed by treatment with enterokinase to provide the purified ActRII polypeptide. See, e.g., Hochuli et al. (1987) J. Chromatography 411:177; and Janknecht et al. (1991) PNAS USA 88:8972.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 4. Methods of Use

In part, the present disclosure relates to methods of treating pulmonary arterial hypertension (PAH) comprising administering to a patient in need thereof an effective amount of an ActRII polypeptide as described herein. In some embodiments, the disclosure contemplates methods of treating, preventing, or reducing the progression rate and/or severity of one or more complications of pulmonary arterial hypertension, comprising administering to a patient in need thereof an effective amount of an ActRII polypeptide as described herein. In some embodiments, the ActRII polypeptide is administered at a dosing range of 0.1 mg/kg to 2.0 mg/kg (e.g., 0.3 mg/kg or 0.7 mg/kg). In some embodiments, the administration of an ActRII polypeptide results in a change of one or more hemodynamic or functional parameters (e.g., a reduction in pulmonary vascular resistance (PVR); an increase in 6-minute walk distance (6 MWD); a decrease of the N-terminal pro B-type natriuretic peptide (NT-proBNP) levels; a prevention or delay in pulmonary hypertension Functional Class progression as recognized by the World Health Organization (WHO); a promotion or increase in pulmonary hypertension Functional Class regression as recognized by the WHO; an improvement in right ventricular function; and an improvement in pulmonary artery pressure).

These methods are particularly aimed at therapeutic and prophylactic treatments of animals, and more particularly, humans. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The terms “treatment”, “treating”, “alleviating” and the like are used herein to generally mean obtaining a desired pharmacologic and/or physiologic effect, and may also be used to refer to improving, alleviating, and/or decreasing the severity of one or more clinical complication of a condition being treated (e.g., PAH). The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure (e.g., PAH) is achieved by administering one or more ActRII polypeptides of the present disclosure in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

In certain aspects, the disclosure contemplates the use of an ActRII polypeptide, in combination with one or more additional active agents or other supportive therapy for treating or preventing a disease or condition (e.g., PAH). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more ActRII polypeptides of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the ActRII polypeptide of the present disclosure with the additional active agent or therapy and/or the desired effect.

### WHO Classification Outline

A pulmonary arterial hypertension condition treated by methods describe herein, can comprise any one or more of the conditions recognized according to the World Health Organization (WHO). See, e.g., Simonneau (2019) Eur Respir J: 53:1801913.

TABLE 1
Clinical Classification of Pulmonary Arterial Hypertension
Group 1: Pulmonary arterial hypertension (PAH)

| | |
|---|---|
| 1.1 | Idiopathic PAH |
| 1.2 | Heritable PAH |
| | 1.2.1 BMPR2 |
| | 1.2.2 ALK-1, ENG, SMAD9, CAV1, KCNK3 |
| | 1.2.3 Unknown |
| 1.3 | Drug and toxin induced PAH |
| 1.4 | Associated with: |
| | 1.4.1 Connective tissue disease |
| | 1.4.2 HIV infection |
| | 1.4.3 Portal hypertension |
| | 1.4.4 Congenital heart diseases |
| | 1.4.5 Schistosomiasis |
| 1.5 | PAH long-term responders to calcium channel blockers |
| 1.6 | PAH with overt features of venous/capillaries (PVOD/PCH) |
| | involvement |
| 1.7 | Persistent PH of the newborn syndrome |

The clinical purpose of the classification of PAH is to categorize clinical conditions associated with PAH into specific subgroups according to their pathophysiological mechanisms, clinical presentation, hemodynamic characteristics, and treatment strategy. This clinical classification may be updated when new data are available on the above features or when additional clinical entities are considered.

As used herein, the term “pulmonary hemodynamic parameter” refers to any parameter used to describe or evaluate the blood flow through the heart and pulmonary vasculature. Examples of pulmonary hemodynamic parameters include, but are not limited to, mean pulmonary artery pressure (mPAP), diastolic pulmonary artery pressure (dPAP) [also known as pulmonary artery diastolic pressure (PADP)], systolic pulmonary artery pressure (sPAP) [also known as pulmonary artery systolic pressure (PASP)], mean right atrial pressure (mRAP), pulmonary capillary wedge pressure (PCWP) [also known as pulmonary artery wedge pressure (PAWP)], pulmonary vascular resistance (PVR) and cardiac output (CO).

Many of the pulmonary hemodynamic parameters described above are interrelated. For example, PVR is related to mPAP, PCWP and CO according to the following equation:

$$PVR = ( m PAP - PCWP ) / CO [ Woods Units ]$$

The PVR measures the resistance to flow imposed by the pulmonary vasculature without the influence of the left-sided filling pressure. PVR can also be measured according to the following equations:

$$PVR = TPG \times 80 / CO [ unit : dynes - sec - {cm}^{- 5} ] OR PVR = ( mPAP - PCWP ) \times 80 / CO [ unit : dynes - sec - {cm}^{- 5} ]$$

In some embodiments, the total peripheral resistance (TPR) can be measured using the following equation:

$$TPR = mPAP / CO .$$

According to some embodiments, a pre-capillary pulmonary arterial contribution to PH may be reflected by an elevated PVR. In some embodiments, the normal PVR is 20-130 dynes-sec-cm⁻⁵ or 0.5-1.1 Wood units. According to some embodiments, an elevated PVR may refer to a PVR above 2 Wood units, above 2.5 Wood units, above 3 Wood units or above 3.5 Wood units.

As yet another example, mPAP is related to dPAP and sPAP according to the following equation:

$$m P AP = ( 2 / 3 ) dPAP + ( 1 / 3 ) sPAP$$

Furthermore, dPAP and sPAP can be used to calculate the pulse pressure (mmHg) using the following equation:

$$pulse pressure = sPAP - dPAP$$

Pulse pressure can be used to calculate the pulmonary artery compliance using the following equation:

$$pulmonary artery compliance ( mL mm {Hg}^{- 1} ) = stroke volume / pulse pressure$$

In some embodiments, the pulmonary hemodynamic parameters are measured directly, such as during a right heart catheterization. In other embodiments, the pulmonary hemodynamic parameters are estimated and/or evaluated through other techniques such as magnetic resonance imaging (MRI) or echocardiography.

Exemplary pulmonary hemodynamic parameters include mPAP, PAWP, and PVR. The one or more pulmonary hemodynamic parameters may be measured by any appropriate procedures, such as by utilizing a right heart catheterization or echocardiography. Various hemodynamic characteristics of PH and PAH are shown in Table 2.

TABLE 2
Hemodynamic Characteristics of Pulmonary Hypertension (PH) and PAH

| | Hemodynamic Characteristics |
|---|---|
| Pulmonary | mPAP >20 mmHg |
| Hypertension | |
| | mPAP >20 mmHg |
| | PAWP ≤15 mmHg |
| Pulmonary | PVR ≥3 Wood units |
| arterial | |
| hypertension | |

The clinical classification or hemodynamic characteristics of PAH described herein and the associated diagnostic parameters may be updated or varied based on the availability of new or existing sources of data or when additional clinical entities are considered.

### Characteristics of PAH

Pulmonary arterial hypertension (WHO Group 1 PH) is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary artery pressures. These high pressures make it difficult for the heart to pump blood through the lungs to be oxygenated. Patients with PAH suffer from extreme shortness of breath as the heart struggles to pump against these high pressures. Patients with PAH typically develop significant increases in PVR and sustained elevations in mPAP, which ultimately lead to right ventricular failure and death. Patients diagnosed with PAH have a poor prognosis and equally compromised quality of life, with a mean life expectancy of 2 to 5 years from the time of diagnosis if untreated.

A variety of factors contribute to the pathogenesis of pulmonary hypertension including proliferation of pulmonary cells which can contribute to vascular remodeling (i.e., hyperplasia). For example, pulmonary vascular remodeling occurs primarily by proliferation of arterial endothelial cells and smooth muscle cells of patients with pulmonary hypertension. Overexpression of various cytokines is believed to promote pulmonary hypertension. Further, it has been found that pulmonary hypertension may rise from the hyperproliferation of pulmonary arterial smooth cells and pulmonary endothelial cells. Still further, advanced PAH may be characterized by muscularization of distal pulmonary arterioles, concentric intimal thickening, and obstruction of the vascular lumen by proliferating endothelial cells. Pietra et al., J. Am. Coll. Cardiol., 43:255-325 (2004).

PAH can be diagnosed based on a mean pulmonary artery pressure of above 25 mmHg (or above 20 mmHg under updated guidelines) at rest, with a normal pulmonary artery capillary wedge pressure. PAH can lead to shortness of breath, dizziness, fainting, and other symptoms, all of which are exacerbated by exertion. PAH can be a severe disease with a markedly decreased exercise tolerance and heart failure. Two major types of PAH include idiopathic PAH (e.g., PAH in which no predisposing factor is identified) and heritable PAH (e.g., PAH associated with a mutation in BMPR2, ALK1, ENG, SMAD9, CAV1, KCNK3, or EIF2AK4). In 70% of familial PAH cases, mutations are located in the BMPR2 gene. Risk factors for the development of PAH include family history of PAH, drug and toxin use (e.g., methamphetamine or cocaine use), infection (e.g., HIV infection or schistosomiasis), cirrhosis of the liver, congenital heart abnormalities, portal hypertension, pulmonary veno-occlusive disease, pulmonary capillary hemangiomatosis, or connective tissue/autoimmune disorders (e.g., scleroderma or lupus). PAH may be associated with long term responders to calcium channel blockers, overt features of venous/capillaries (PVOD/PCH) involvement, and persistent PH of the newborn syndrome.

### Diagnosis of PAH

The diagnosis of PAH, including functional group, can be determined based on symptoms and physical examination using a review of a comprehensive set of parameters to determine if the hemodynamic and other criteria are met. Some of the criteria which may considered include the patient's clinical presentation (e.g., shortness of breath, fatigue, weakness, angina, syncope, dry-couch, exercise-induced nausea and vomiting), electrocardiogram (ECG) results, chest radiograph results, pulmonary function tests, arterial blood gases, echocardiography results, ventilation/perfusion lung scan results, high-resolution computed tomography results, contrast-enhanced computed tomography results, pulmonary angiography results, cardiac magnetic resonance imaging, blood tests (e.g., biomarkers such as BNP or NT-proBNP), immunology, abdominal ultrasound scan, right heart catherization (RHC), vasoreactivity, and genetic testing. See, e.g., Galie N., et al Euro Heart J. (2016) 37, 67-119.

In some embodiments, a biomarker may be used to aid in the diagnosis of PAH. For instance, in some embodiments, the biomarker is a marker of vascular dysfunction (e.g., asymmetric dimethylarginine (ADMA), endothelin-1, angiopoeitins, or von Willebrand factor). In some embodiments, the biomarker is a marker of inflammation (C-reactive protein, interleukin 6, chemokines). In some embodiments, the biomarker is a marker of myocardial stress [e.g., (atrial natriuretic peptide, brain natriuretic peptide (BNP)/NT-proBNP, or troponins]. In some embodiments, the biomarker is a marker of low CO and/or tissue hypoxia (e.g., pCO2, uric acid, growth differentiation factor 15 (GDF15), or osteopontin). In some embodiments, the biomarker is a marker of secondary organ damage (e.g., creatinine or bilirubin). See, e.g., Galie N., et al Euro Heart J. (2016) 37, 67-119.

### Measurements of PH

In certain aspects, the disclosure relates to methods of treating, preventing, or reducing the progression rate and/or severity of pulmonary arterial hypertension (PAH) comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to treating PAH patients that have idiopathic PAH. In some embodiments, the method relates to treating PAH patients that have heritable PAH (e.g., PAH due to one or more mutations within BMPR2, ALK-1, ENG, SMAD9, CAV1, and KCNK3). In some embodiments, the method relates to treating PAH patients that have heritable PAH due to an unknown mutation. In some embodiments, the method relates to treating PAH patients that have drug or toxin induced PAH. In some embodiments, the method relates to treating PAH patients that have PAH associated with connective tissue disease. In some embodiments, the method relates to treating PAH patients that have PAH associated with HIV infection. In some embodiments, the method relates to treating PAH patients that have PAH associated with portal hypertension. In some embodiments, the method relates to treating PAH patients that have PAH associated with schistosomiasis. In some embodiments, the method relates to treating PAH patients classified as long-term responders to calcium channel blockers. In some embodiments, the method relates to treating PAH patients with overt features of venous/capillaries (PVOD/PCH) involvement. In some embodiments, the method relates to treating PAH patients that have persistent pulmonary hypertension (PH) of the newborn syndrome. In some embodiments, the method relates to treating PAH patients that have PAH associated with simple, congenital systemic-to-pulmonary shunts at least 1 year following shunt repair.

mPAP

In certain aspects, the disclosure relates to methods of treating, preventing, or reducing the progression rate and/or severity of pulmonary arterial hypertension (PAH) comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has a resting mean pulmonary artery pressure (mPAP) of at least 20 mmHg (e.g., 20, 25, 30, 35, 40, 45, or 50 mmHg). In some embodiments, the method relates to patients having a resting mPAP of at least 20 mmHg. In some embodiments, the method relates to patients having a resting mPAP of at least 25 mmHg. In some embodiments, the method relates to patients having a resting mPAP of at least 30 mmHg. In some embodiments, the method relates to patients having a resting mPAP of at least 35 mmHg. In some embodiments, the method relates to patients having a resting mPAP of at least 40 mmHg. In some embodiments, the method relates to patients having a resting mPAP of at least 45 mmHg. In some embodiments, the method relates to patients having a resting mPAP of at least 50 mmHg.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to improving the pulmonary artery pressure in the patient. In some embodiments, the improvement in pulmonary artery pressure is a reduction in the mean pulmonary artery pressure (mPAP). In some embodiments, the method relates to reducing mPAP. In some embodiments, the method relates to reducing the patient's mPAP by at least 1 mmHg. In some embodiments, the method relates to reducing the patient's mPAP by at least 2 mmHg. In some embodiments, the method relates to reducing the patient's mPAP by at least 3 mmHg. In certain embodiments, the method relates to reducing the patient's mPAP by at least 5 mmHg. In certain embodiments, the method relates to reducing the patient's mPAP by at least 7 mmHg. In certain embodiments, the method relates to reducing the patient's mPAP by at least 10 mmHg. In certain embodiments, the method relates to reducing the patient's mPAP by at least 12 mmHg. In certain embodiments, the method relates to reducing the patient's mPAP by at least 15 mmHg. In certain embodiments, the method relates to reducing the patient's mPAP by at least 20 mmHg. In certain embodiments, the method relates to reducing the patient's mPAP by at least 25 mmHg.

In some embodiments, the disclosure relates to methods of adjusting one or more hemodynamic parameters in the PH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to decreasing the patient's mPAP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's mPAP by at least 1%. In some embodiments, the method relates to decreasing the patient's mPAP by at least 5%. In some embodiments, the method relates to decreasing the patient's mPAP by at least 10%. In some embodiments, the method relates to decreasing the patient's mPAP by at least 15%. In some embodiments, the method relates to decreasing the patient's mPAP by at least 20%. In some embodiments, the method relates to decreasing the patient's mPAP by at least 25%. In some embodiments, the method relates to decreasing the patient's mPAP by at least 30%. In some embodiments, the method relates to decreasing the patient's mPAP by at least 35%. In some embodiments, the method relates to decreasing the patient's mPAP by at least 40%. In some embodiments, the method relates to decreasing the patient's mPAP by at least 45%. In some embodiments, the method relates to decreasing the patient's mPAP by at least 50%. In some embodiments, the method relates to decreasing the patient's mPAP by at least 55%. In some embodiments, the method relates to decreasing the patient's mPAP by at least 60%. In some embodiments, the method relates to decreasing the patient's mPAP by at least 65%. In some embodiments, the method relates to decreasing the patient's mPAP by at least 70%. In some embodiments, the method relates to decreasing the patient's mPAP by at least 75%. In some embodiments, the method relates to decreasing the patient's mPAP by at least 80%. In some embodiments, the method relates to decreasing the patient's mPAP by at least 85%. In some embodiments, the method relates to decreasing the patient's mPAP by at least 90%. In some embodiments, the method relates to decreasing the patient's mPAP by at least 95%. In some embodiments, the method relates to decreasing the patient's mPAP by at least 100%.

mRAP

As PAH progresses, increased pulmonary vascular resistance to blood flow leads to increased right atrial pressure (RAP) and right heart failure. Patients with right heart failure typically have an increased ratio of right atrial pressure (RAP) and pulmonary artery wedge pressure (PAWP). In certain aspects, the disclosure relates to methods of treating, preventing, or reducing the progression rate and/or severity of pulmonary arterial hypertension (PAH) comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has resting mean right atrial pressure (mRAP) of at least 5 mmHg (e.g., at least 5, 6, 7, 8, 9, 10, 11, 12, 13, 14, 15, 16, 17, 18, 18, 20, 21, 22, 23, 24, or 25 mmHg). In some embodiments, the method relates to patients having a resting mRAP of at least 5 mmHg. In some embodiments, the method relates to patients having a resting mRAP of at least 6 mmHg. In some embodiments, the method relates to patients having a resting mRAP of at least 7 mmHg. In some embodiments, the method relates to patients having a resting mRAP of at least 8 mmHg. In some embodiments, the method relates to patients having a resting mRAP of at least 9 mmHg. In some embodiments, the method relates to patients having a resting mRAP of at least 10 mmHg. In some embodiments, the method relates to patients having a resting mRAP of at least 11 mmHg. In some embodiments, the method relates to patients having a resting mRAP of at least 12 mmHg. In some embodiments, the method relates to patients having a resting mRAP of at least 13 mmHg. In some embodiments, the method relates to patients having a resting mRAP of at least 14 mmHg. In some embodiments, the method relates to patients having a resting mRAP of at least 15 mmHg. In some embodiments, the method relates to patients having a resting mRAP of at least 16 mmHg. In some embodiments, the method relates to patients having a resting mRAP of at least 17 mmHg. In some embodiments, the method relates to patients having a resting mRAP of at least 18 mmHg. In some embodiments, the method relates to patients having a resting mRAP of at least 19 mmHg. In some embodiments, the method relates to patients having a resting mRAP of at least 20 mmHg. In some embodiments, the method relates to patients having a resting mRAP of at least 21 mmHg. In some embodiments, the method relates to patients having a resting mRAP of at least 22 mmHg. In some embodiments, the method relates to patients having a resting mRAP of at least 23 mmHg. In some embodiments, the method relates to patients having a resting mRAP of at least 24 mmHg. In some embodiments, the method relates to patients having a resting mRAP of at least 25 mmHg.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to improving the mean right atrial pressure in the patient. In some embodiments, the improvement in the mean right atrial pressure (mRAP) is a reduction in the mRAP. In some embodiments, the method relates to reducing mRAP. In some embodiments, the method relates to reducing the patient's mRAP by at least 1 mmHg. In some embodiments, the method relates to reducing the patient's mRAP by at least 2 mmHg. In some embodiments, the method relates to reducing the patient's mRAP by at least 3 mmHg. In some embodiments, the method relates to reducing the patient's mRAP by at least 4 mmHg. In certain embodiments, the method relates to reducing the patient's mRAP by at least 5 mmHg. In some embodiments, the method relates to reducing the patient's mRAP by at least 6 mmHg. In certain embodiments, the method relates to reducing the patient's mRAP by at least 7 mmHg. In some embodiments, the method relates to reducing the patient's mRAP by at least 8 mmHg. In some embodiments, the method relates to reducing the patient's mRAP by at least 9 mmHg. In certain embodiments, the method relates to reducing the patient's mRAP by at least 10 mmHg. In some embodiments, the method relates to reducing the patient's mRAP by at least 11 mmHg. In certain embodiments, the method relates to reducing the patient's mRAP by at least 12 mmHg. In some embodiments, the method relates to reducing the patient's mRAP by at least 13 mmHg. In some embodiments, the method relates to reducing the patient's mRAP by at least 14 mmHg. In certain embodiments, the method relates to reducing the patient's mRAP by at least 15 mmHg. In some embodiments, the method relates to reducing the patient's mRAP by at least 16 mmHg. In some embodiments, the method relates to reducing the patient's mRAP by at least 17 mmHg. In some embodiments, the method relates to reducing the patient's mRAP by at least 18 mmHg. In some embodiments, the method relates to reducing the patient's mRAP by at least 19 mmHg. In certain embodiments, the method relates to reducing the patient's mRAP by at least 20 mmHg.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to decreasing the patient's mRAP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's mRAP by at least 1%. In some embodiments, the method relates to decreasing the patient's mRAP by at least 5%. In some embodiments, the method relates to decreasing the patient's mRAP by at least 10%. In some embodiments, the method relates to decreasing the patient's mRAP by at least 15%. In some embodiments, the method relates to decreasing the patient's mRAP by at least 20%. In some embodiments, the method relates to decreasing the patient's mRAP by at least 25%. In some embodiments, the method relates to decreasing the patient's mRAP by at least 30%. In some embodiments, the method relates to decreasing the patient's mRAP by at least 35%. In some embodiments, the method relates to decreasing the patient's mRAP by at least 40%. In some embodiments, the method relates to decreasing the patient's mRAP by at least 45%. In some embodiments, the method relates to decreasing the patient's mRAP by at least 50%. In some embodiments, the method relates to decreasing the patient's mRAP by at least 55%. In some embodiments, the method relates to decreasing the patient's mRAP by at least 60%. In some embodiments, the method relates to decreasing the patient's mRAP by at least 65%. In some embodiments, the method relates to decreasing the patient's mRAP by at least 70%. In some embodiments, the method relates to decreasing the patient's mRAP by at least 75%. In some embodiments, the method relates to decreasing the patient's mRAP by at least 80%. In some embodiments, the method relates to decreasing the patient's mRAP by at least 85%. In some embodiments, the method relates to decreasing the patient's mRAP by at least 90%. In some embodiments, the method relates to decreasing the patient's mRAP by at least 95%. In some embodiments, the method relates to decreasing the patient's mRAP by at least 100%.

### PVR

In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has a pulmonary vascular resistance (PVR) of at least 2.5 Woods Units (e.g., at least 2.5, 3, 4, 5, 6, 7, 8, 9, 10, 12, 14, 16, 18, or 20 Woods Units). In some embodiments, the method relates to patients having a PVR of at least 2.5 Woods Units. In some embodiments, the method relates to patients having a PVR of at least 3 Woods Units. In some embodiments, the method relates to patients having a PVR of at least 4 Woods Units. In some embodiments, the method relates to patients having a PVR of at least 5 Woods Units. In some embodiments, the method relates to patients having a PVR of at least 6 Woods Units. In some embodiments, the method relates to patients having a PVR of at least 7 Woods Units. In some embodiments, the method relates to patients having a PVR of at least 8 Woods Units. In some embodiments, the method relates to patients having a PVR of at least 9 Woods Units. In some embodiments, the method relates to patients having a PVR of at least 10 Woods Units. In some embodiments, the method relates to patients having a PVR of at least 12 Woods Units. In some embodiments, the method relates to patients having a PVR of at least 14 Woods Units. In some embodiments, the method relates to patients having a PVR of at least 16 Woods Units. In some embodiments, the method relates to patients having a PVR of at least 18 Woods Units. In some embodiments, the method relates to patients having a PVR of at least 20 Woods Units.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to reducing the patient's PVR. In some embodiments, the reduction in the patient's PVR is a result of a decrease in the patient's mean pulmonary artery pressure (mPAP). In some embodiments, the method relates to reducing the patient's PVR by at least 0.5 Wood Units. In some embodiments, the method relates to reducing the patient's PVR by at least 1 Wood Units. In some embodiments, the method relates to reducing the patient's PVR by at least 2 Wood Units. In some embodiments, the method relates to reducing the patient's PVR by at least 4 Wood Units. In some embodiments, the method relates to reducing the patient's PVR by at least 6 Wood Units. In some embodiments, the method relates to reducing the patient's PVR by at least 8 Wood Units. In some embodiments, the method relates to reducing the patient's PVR by at least 10 Wood Units.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to decreasing the patient's PVR by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's PVR. In some embodiments, the decrease in the patient's PVR is a result of a decrease in the patient's mean pulmonary artery pressure (nmPAP). In some embodiments, the method relates to decreasing the patient's PVR by at least 1%. In some embodiments, the method relates to decreasing the patient's PVR by at least 5%. In some embodiments, the method relates to decreasing the patient's PVR by at least 10%. In some embodiments, the method relates to decreasing the patient's PVR by at least 15%. In some embodiments, the method relates to decreasing the patient's PVR by at least 20%. In some embodiments, the method relates to decreasing the patient's PVR by at least 25%. In some embodiments, the method relates to decreasing the patient's PVR by at least 30%. In some embodiments, the method relates to decreasing the patient's PVR by at least 35%. In some embodiments, the method relates to decreasing the patient's PVR by at least 40%. In some embodiments, the method relates to decreasing the patient's PVR by at least 45%. In some embodiments, the method relates to decreasing the patient's PVR by at least 50%. In some embodiments, the method relates to decreasing the patient's PVR by at least 55%. In some embodiments, the method relates to decreasing the patient's PVR by at least 60%. In some embodiments, the method relates to decreasing the patient's PVR by at least 65%. In some embodiments, the method relates to decreasing the patient's PVR by at least 70%. In some embodiments, the method relates to decreasing the patient's PVR by at least 75%. In some embodiments, the method relates to decreasing the patient's PVR by at least 80%. In some embodiments, the method relates to decreasing the patient's PVR by at least 85%. In some embodiments, the method relates to decreasing the patient's PVR by at least 90%. In some embodiments, the method relates to decreasing the patient's PVR by at least 95%. In some embodiments, the method relates to decreasing the patient's PVR by at least 100%.

In some embodiments, PVR is tested after the patient has received 4 weeks of treatment utilizing an ActRII polypeptide disclosed herein. In some embodiments, PVR is tested after the patient has received 8 weeks of treatment utilizing an ActRII polypeptide disclosed herein. In some embodiments, PVR is tested after the patient has received 12 weeks of treatment utilizing an ActRII polypeptide disclosed herein. In some embodiments, PVR is tested after the patient has received 16 weeks of treatment utilizing an ActRII polypeptide disclosed herein. In some embodiments, PVR is tested after the patient has received 20 weeks of treatment utilizing an ActRII polypeptide disclosed herein. In some embodiments, PVR is tested after the patient has received 22 weeks of treatment utilizing an ActRII polypeptide disclosed herein. In some embodiments, PVR is tested after the patient has received 24 weeks of treatment utilizing an ActRII polypeptide disclosed herein. In some embodiments, PVR is tested after the patient has received 26 weeks of treatment utilizing an ActRII polypeptide disclosed herein. In some embodiments, PVR is tested after the patient has received 28 weeks of treatment utilizing an ActRII polypeptide disclosed herein. In some embodiments, PVR is tested after the patient has received 48 weeks of treatment utilizing an ActRII polypeptide disclosed herein.

### BNP

Both BNP and NT-proBNP are markers of atrial and ventricular distension due to increased intracardiac pressure. The New York Heart Association (NYHA) developed a 4-stage functional classification system for congestive heart failure (CHF) based on the severity of symptoms. Studies have demonstrated that the measured concentrations of circulating BNP and NT-proBNP increase with the severity of CHF based on the NYHA classification. In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has a brain natriuretic peptide (BNP) level of at least 100 pg/mL (e.g., at least 100, 150, 200, 300, 400, 500, 600, 700, 800, 900, 1000, 3000, 5000, 10,000, 15,000, or 20,000 pg/mL). In some embodiments, the method relates to patient's having a BNP level of at least 100 pg/mL. In some embodiments, the method relates to patient's having a BNP level of at least 150 pg/mL. In some embodiments, the method relates to patient's having a BNP level of at least 200 pg/mL. In some embodiments, the method relates to patient's having a BNP level of at least 300 pg/mL. In some embodiments, the method relates to patient's having a BNP level of at least 400 pg/mL. In some embodiments, the method relates to patient's having a BNP level of at least 500 pg/mL. In some embodiments, the method relates to patient's having a BNP level of at least 600 pg/mL. In some embodiments, the method relates to patient's having a BNP level of at least 700 pg/mL. In some embodiments, the method relates to patient's having a BNP level of at least 800 pg/mL. In some embodiments, the method relates to patient's having a BNP level of at least 900 pg/mL. In some embodiments, the method relates to patient's having a BNP level of at least 1000 pg/mL. In some embodiments, the method relates to patient's having a BNP level of at least 3000 pg/mL. In some embodiments, the method relates to patient's having a BNP level of at least 5000 pg/mL. In some embodiments, the method relates to patient's having a BNP level of at least 10,000 pg/mL. In some embodiments, the method relates to patient's having a BNP level of at least 15,000 pg/mL. In some embodiments, the method relates to patient's having a BNP level of at least 20,000 pg/mL. In some embodiments, the method relates to treatment of a patient who has elevated BNP levels as compared to a healthy patient.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to reducing the patient's BNP levels by at least 10 pg/mL. In some embodiments, the method relates to reducing the patient's BNP levels by at least 50 pg/mL. In some embodiments, the method relates to reducing the patient's BNP levels by at least 100 pg/mL. In some embodiments, the method relates to reducing the patient's BNP levels by at least 200 pg/mL. In some embodiments, the method relates to reducing the patient's BNP levels by at least 300 pg/mL. In some embodiments, the method relates to reducing the patient's BNP levels by at least 400 pg/mL. In some embodiments, the method relates to reducing the patient's BNP levels by at least 500 pg/mL. In some embodiments, the method relates to reducing the patient's BNP levels by at least 600 pg/mL. In some embodiments, the method relates to reducing the patient's BNP levels by at least 700 pg/mL. In some embodiments, the method relates to reducing the patient's BNP levels by at least 800 pg/mL. In some embodiments, the method relates to reducing the patient's BNP levels by at least 900 pg/mL. In some embodiments, the method relates to reducing the patient's BNP levels by at least 1000 pg/mL. In some embodiments, the method relates to reducing the patient's BNP levels by at least 5000 pg/mL. In some embodiments, the method relates to reducing the patient's BNP levels to normal levels. In some embodiments, normal levels correspond to levels of <100 pg/mL.

In some embodiments, the method relates to reducing the patient's BNP by at least 5% (e.g., at least 5, 10, 15, 20, 25, 30, 35, 40, 45, 50, 55, 60, 65, 70, 75, 80, 85, 90, 95, or 100%). In some embodiments, the method relates to reducing the patient's BNP by at least 5%. In some embodiments, the method relates to reducing the patient's BNP by at least 10%. In some embodiments, the method relates to reducing the patient's BNP by at least 15%. In some embodiments, the method relates to reducing the patient's BNP by at least 20%. In some embodiments, the method relates to reducing the patient's BNP by at least 25%. In some embodiments, the method relates to reducing the patient's BNP by at least 30%. In some embodiments, the method relates to reducing the patient's BNP by at least 35%. In some embodiments, the method relates to reducing the patient's BNP by at least 40%. In some embodiments, the method relates to reducing the patient's BNP by at least 45%. In some embodiments, the method relates to reducing the patient's BNP by at least 50%. In some embodiments, the method relates to reducing the patient's BNP by at least 55%. In some embodiments, the method relates to reducing the patient's BNP by at least 60%. In some embodiments, the method relates to reducing the patient's BNP by at least 65%. In some embodiments, the method relates to reducing the patient's BNP by at least 70%. In some embodiments, the method relates to reducing the patient's BNP by at least 75%. In some embodiments, the method relates to reducing the patient's BNP by at least 80%. In some embodiments, the method relates to reducing the patient's BNP by at least 85%. In some embodiments, the method relates to reducing the patient's BNP by at least 90%. In some embodiments, the method relates to reducing the patient's BNP by at least 95%. In some embodiments, the method relates to reducing the patient's BNP by at least 100%.

### NT-proBNP

In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has a NT-proBNP level of at least 100 pg/mL (e.g., at least 100, 150, 200, 300, 400, 500, 600, 700, 800, 900, 1000, 3000, 5000, 10,000, 15,000, 20,000, 25,000, or 30,000 pg/mL). In some embodiments, the method relates to patient's having a NT-proBNP level of at least 100 pg/mL. In some embodiments, the method relates to patient's having a NT-proBNP level of at least 150 pg/mL. In some embodiments, the method relates to patient's having a NT-proBNP level of at least 200 pg/mL. In some embodiments, the method relates to patient's having a NT-proBNP level of at least 300 pg/mL. In some embodiments, the method relates to patient's having a NT-proBNP level of at least 400 pg/mL. In some embodiments, the method relates to patient's having a NT-proBNP level of at least 500 pg/mL. In some embodiments, the method relates to patient's having a NT-proBNP level of at least 600 pg/mL. In some embodiments, the method relates to patient's having a NT-proBNP level of at least 700 pg/mL. In some embodiments, the method relates to patient's having a NT-proBNP level of at least 800 pg/mL. In some embodiments, the method relates to patient's having a NT-proBNP level of at least 900 pg/mL. In some embodiments, the method relates to patient's having a NT-proBNP level of at least 1000 pg/mL. In some embodiments, the method relates to patient's having a NT-proBNP level of at least 3000 pg/mL. In some embodiments, the method relates to patient's having a NT-proBNP level of at least 5000 μg/mL. In some embodiments, the method relates to patient's having a NT-proBNP level of at least 10,000 pg/mL. In some embodiments, the method relates to patient's having a NT-proBNP level of at least 15,000 pg/mL. In some embodiments, the method relates to patient's having a NT-proBNP level of at least 20,000 pg/mL. In some embodiments, the method relates to patient's having a NT-proBNP level of at least 25,000 pg/mL. In some embodiments, the method relates to patient's having a NT-proBNP level of at least 30,000 pg/mL. In some embodiments, the method relates to treatment of a patient who has elevated NT-proBNP levels as compared to a healthy patient.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to decreasing the patient's NT-proBNP levels. In some embodiments, the method relates to decreasing the patient's NT-proBNP by at least 10 pg/mL. In some embodiments, the method relates to decreasing the patient's NT-proBNP by at least 50 pg/mL. In some embodiments, the method relates to decreasing the patient's NT-proBNP by at least 100 pg/mL. In some embodiments, the method relates to decreasing the patient's NT-proBNP by at least 200 pg/mL. In some embodiments, the method relates to decreasing the patient's NT-proBNP by at least 300 pg/mL. In some embodiments, the method relates to decreasing the patient's NT-proBNP by at least 400 pg/mL. In some embodiments, the method relates to decreasing the patient's NT-proBNP by at least 500 pg/mL. In some embodiments, the method relates to decreasing the patient's NT-proBNP by at least 600 pg/mL. In some embodiments, the method relates to decreasing the patient's NT-proBNP by at least 700 pg/mL. In some embodiments, the method relates to decreasing the patient's NT-proBNP by at least 800 pg/mL. In some embodiments, the method relates to decreasing the patient's NT-proBNP by at least 900 pg/mL. In some embodiments, the method relates to decreasing the patient's NT-proBNP by at least 1000 pg/mL. In some embodiments, the method relates to decreasing the patient's NT-proBNP by at least 5000 pg/mL. In some embodiments, the method relates to decreasing the patient's NT-proBNP by at least 10,000 pg/mL. In some embodiments, the method relates to decreasing the patient's NT-proBNP by at least 15,000 pg/mL. In some embodiments, the method relates to decreasing the patient's NT-proBNP by at least 20,000 pg/mL. In some embodiments, the method relates to decreasing the patient's NT-proBNP by at least 25,000 pg/mL.

In some embodiments, the method relates to decreasing the patient's NT-proBNP levels to a normal level and maintain their normal NT-proBNP levels. In some embodiments, the disclosure relates to methods of maintaining one or more hemodynamic parameters in the PAH patient at a normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to maintaining the patient's NT-proBNP levels at a normal level. In some embodiments, the method relates to maintaining the patient's NT-proBNP level at less than 100 pg/mL. In some embodiments, the method relates to maintaining the patient's NT-proBNP level at less than 200 pg/mL. In some embodiments, the method relates to maintaining the patient's NT-proBNP level at less than 300 pg/mL. In some embodiments, the method relates to maintaining the patient's NT-proBNP level at less than 400 pg/mL.

In some embodiments, the method relates to decreasing the patient's NT-proBNP by at least 5% (e.g., at least 5, 10, 15, 20, 25, 30, 35, 40, 45, 50, 55, 60, 65, 70, 75, 80, 85, 90, 95, or 100%). In some embodiments, the method relates to decreasing the patient's NT-proBNP by at least 5%. In some embodiments, the method relates to decreasing the patient's NT-proBNP by at least 10%. In some embodiments, the method relates to decreasing the patient's NT-proBNP by at least 15%. In some embodiments, the method relates to decreasing the patient's NT-proBNP by at least 20%. In some embodiments, the method relates to decreasing the patient's NT-proBNP by at least 25%. In some embodiments, the method relates to decreasing the patient's NT-proBNP by at least 30%. In some embodiments, the method relates to decreasing the patient's NT-proBNP by at least 35%. In some embodiments, the method relates to decreasing the patient's NT-proBNP by at least 40%. In some embodiments, the method relates to decreasing the patient's NT-proBNP by at least 45%. In some embodiments, the method relates to decreasing the patient's NT-proBNP by at least 50%. In some embodiments, the method relates to decreasing the patient's NT-proBNP by at least 55%. In some embodiments, the method relates to decreasing the patient's NT-proBNP by at least 60%. In some embodiments, the method relates to decreasing the patient's NT-proBNP by at least 65%. In some embodiments, the method relates to decreasing the patient's NT-proBNP by at least 70%. In some embodiments, the method relates to decreasing the patient's NT-proBNP by at least 75%. In some embodiments, the method relates to decreasing the patient's NT-proBNP by at least 80%. In some embodiments, the method relates to decreasing the patient's NT-proBNP by at least 85%. In some embodiments, the method relates to decreasing the patient's NT-proBNP by at least 90%. In some embodiments, the method relates to decreasing the patient's NT-proBNP by at least 95%. In some embodiments, the method relates to decreasing the patient's NT-proBNP by at least 100%. In some embodiments, the method relates to decreasing the patient's NT-proBNP levels to normal levels. In some embodiments, normal levels of NT-proBNP is <100 μg/ml. In some embodiments, the method relates to decreasing the patient's NT-proBNP levels to less than 300 ng/L.

### Smooth Muscle Hypertrophy

In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has smooth muscle hypertrophy. In some embodiments, the disclosure relates to methods of adjusting one or more parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to decreasing smooth muscle hypertrophy in the patient. In some embodiments, the method relates to decreasing the patient's smooth muscle hypertrophy by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's smooth muscle hypertrophy by at least 1%. In some embodiments, the method relates to decreasing the patient's smooth muscle hypertrophy by at least 5%. In some embodiments, the method relates to decreasing the patient's smooth muscle hypertrophy by at least 10%. In some embodiments, the method relates to decreasing the patient's smooth muscle hypertrophy by at least 15%. In some embodiments, the method relates to decreasing the patient's smooth muscle hypertrophy by at least 20%. In some embodiments, the method relates to decreasing the patient's smooth muscle hypertrophy by at least 25%. In some embodiments, the method relates to decreasing the patient's smooth muscle hypertrophy by at least 30%. In some embodiments, the method relates to decreasing the patient's smooth muscle hypertrophy by at least 35%. In some embodiments, the method relates to decreasing the patient's smooth muscle hypertrophy by at least 40%. In some embodiments, the method relates to decreasing the patient's smooth muscle hypertrophy by at least 45%. In some embodiments, the method relates to decreasing the patient's smooth muscle hypertrophy by at least 50%. In some embodiments, the method relates to decreasing the patient's smooth muscle hypertrophy by at least 55%. In some embodiments, the method relates to decreasing the patient's smooth muscle hypertrophy by at least 60%. In some embodiments, the method relates to decreasing the patient's smooth muscle hypertrophy by at least 65%. In some embodiments, the method relates to decreasing the patient's smooth muscle hypertrophy by at least 70%. In some embodiments, the method relates to decreasing the patient's smooth muscle hypertrophy by at least 75%. In some embodiments, the method relates to decreasing the patient's smooth muscle hypertrophy by at least 80%. In some embodiments, the method relates to decreasing the patient's smooth muscle hypertrophy by at least 85%. In some embodiments, the method relates to decreasing the patient's smooth muscle hypertrophy by at least 90%. In some embodiments, the method relates to decreasing the patient's smooth muscle hypertrophy by at least 95%. In some embodiments, the method relates to decreasing the patient's smooth muscle hypertrophy by at least 100%.

### Pulmonary Arteriole Muscularity

In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has increased pulmonary arteriole muscularity. In some embodiments, the disclosure relates to methods of adjusting one or more parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to decreasing pulmonary arteriole muscularity in the patient. In some embodiments, the method relates to decreasing the patient's pulmonary arteriole muscularity by least 1% (e.g., 1, 5, 10, 15, 20, 25, 30, 35, 40, 45, 50, 55, 60, 65, 70, 75, 80, 85, 90, 95, or 100%). In some embodiments, the method relates to decreasing the patient's pulmonary arteriole muscularity by at least 1%. In some embodiments, the method relates to decreasing the patient's pulmonary arteriole muscularity by at least 5%. In some embodiments, the method relates to decreasing the patient's pulmonary arteriole muscularity by at least 10%. In some embodiments, the method relates to decreasing the patient's pulmonary arteriole muscularity by at least 15%. In some embodiments, the method relates to decreasing the patient's pulmonary arteriole muscularity by at least 20%. In some embodiments, the method relates to decreasing the patient's pulmonary arteriole muscularity by at least 25%. In some embodiments, the method relates to decreasing the patient's pulmonary arteriole muscularity by at least 30%. In some embodiments, the method relates to decreasing the patient's pulmonary arteriole muscularity by at least 35%. In some embodiments, the method relates to decreasing the patient's pulmonary arteriole muscularity by at least 40%. In some embodiments, the method relates to decreasing the patient's pulmonary arteriole muscularity by at least 45%. In some embodiments, the method relates to decreasing the patient's pulmonary arteriole muscularity by at least 50%. In some embodiments, the method relates to decreasing the patient's pulmonary arteriole muscularity by at least 55%. In some embodiments, the method relates to decreasing the patient's pulmonary arteriole muscularity by at least 60%. In some embodiments, the method relates to decreasing the patient's pulmonary arteriole muscularity by at least 65%. In some embodiments, the method relates to decreasing the patient's pulmonary arteriole muscularity by at least 70%. In some embodiments, the method relates to decreasing the patient's pulmonary arteriole muscularity by at least 75%. In some embodiments, the method relates to decreasing the patient's pulmonary arteriole muscularity by at least 80%. In some embodiments, the method relates to decreasing the patient's pulmonary arteriole muscularity by at least 85%. In some embodiments, the method relates to decreasing the patient's pulmonary arteriole muscularity by at least 90%. In some embodiments, the method relates to decreasing the patient's pulmonary arteriole muscularity by at least 95%. In some embodiments, the method relates to decreasing the patient's pulmonary arteriole muscularity by at least 100%.

### Rate of Hospitalization

In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the method reduces the patient's hospitalization rate by at least 1% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the method relates to reducing the patient's hospitalization rate by at least 1%. In some embodiments, the method relates to reducing the patient's hospitalization rate by at least 2%. In some embodiments, the method relates to reducing the patient's hospitalization rate by at least 3%. In some embodiments, the method relates to reducing the patient's hospitalization rate by at least 4%. In some embodiments, the method relates to reducing the patient's hospitalization rate by at least 5%. In some embodiments, the method relates to reducing the patient's hospitalization rate by at least 10%. In some embodiments, the method relates to reducing the patient's hospitalization rate by at least 15%. In some embodiments, the method relates to reducing the patient's hospitalization rate by at least 20%. In some embodiments, the method relates to reducing the patient's hospitalization rate by at least 25%. In some embodiments, the method relates to reducing the patient's hospitalization rate by at least 30%. In some embodiments, the method relates to reducing the patient's hospitalization rate by at least 35%. In some embodiments, the method relates to reducing the patient's hospitalization rate by at least 40%. In some embodiments, the method relates to reducing the patient's hospitalization rate by at least 45%. In some embodiments, the method relates to reducing the patient's hospitalization rate by at least 50%. In some embodiments, the method relates to reducing the patient's hospitalization rate by at least 55%. In some embodiments, the method relates to reducing the patient's hospitalization rate by at least 60%. In some embodiments, the method relates to reducing the patient's hospitalization rate by at least 65%. In some embodiments, the method relates to reducing the patient's hospitalization rate by at least 70%. In some embodiments, the method relates to reducing the patient's hospitalization rate by at least 75%. In some embodiments, the method relates to reducing the patient's hospitalization rate by at least 80%. In some embodiments, the method relates to reducing the patient's hospitalization rate by at least 85%. In some embodiments, the method relates to reducing the patient's hospitalization rate by at least 90%. In some embodiments, the method relates to reducing the patient's hospitalization rate by at least 95%. In some embodiments, the method relates to reducing the patient's hospitalization rate by at least 100%. In some embodiments, the method reduces the risk of hospitalization for one or more complications associated with PAH.

### Quality of Life

In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the method increases the patient's quality of life by at least 1% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the method relates to increasing the patient's quality of life by at least 1%. In some embodiments, the method relates to increasing the patient's quality of life by at least 2%. In some embodiments, the method relates to increasing the patient's quality of life by at least 3%. In some embodiments, the method relates to increasing the patient's quality of life by at least 4%. In some embodiments, the method relates to increasing the patient's quality of life by at least 5%. In some embodiments, the method relates to increasing the patient's quality of life by at least 10%. In some embodiments, the method relates to increasing the patient's quality of life by at least 15%. In some embodiments, the method relates to increasing the patient's quality of life by at least 20%. In some embodiments, the method relates to increasing the patient's quality of life by at least 25%. In some embodiments, the method relates to increasing the patient's quality of life by at least 30%. In some embodiments, the method relates to increasing the patient's quality of life by at least 35%. In some embodiments, the method relates to increasing the patient's quality of life by at least 40%. In some embodiments, the method relates to increasing the patient's quality of life by at least 45%. In some embodiments, the method relates to increasing the patient's quality of life by at least 50%. In some embodiments, the method relates to increasing the patient's quality of life by at least 55%. In some embodiments, the method relates to increasing the patient's quality of life by at least 60%. In some embodiments, the method relates to increasing the patient's quality of life by at least 65%. In some embodiments, the method relates to increasing the patient's quality of life by at least 70%. In some embodiments, the method relates to increasing the patient's quality of life by at least 75%. In some embodiments, the method relates to increasing the patient's quality of life by at least 80%. In some embodiments, the method relates to increasing the patient's quality of life by at least 85%. In some embodiments, the method relates to increasing the patient's quality of life by at least 90%. In some embodiments, the method relates to increasing the patient's quality of life by at least 95%. In some embodiments, the method relates to increasing the patient's quality of life by at least 100%.

In some embodiments, the patient's quality of life is measured using the Cambridge Pulmonary Hypertension Outcome Review (CAMPHOR). In some embodiments, the patient's quality of life is measured using PAH-SYMPACT®. In some embodiments, the patient's quality of life is measured using the Medical Outcomes Survey Short Form-36 (SF-36). In some embodiments, the patient's quality of life is measured using the Euro Quality of Life (EuroQol). In some embodiments, the patient's quality of life is measured using the Euro Quality of Life—5 dimensions (EQ-5D). In some embodiments, the patient's quality of life is measured using the Euro Quality of Life—5 dimensions 5-levels (EQ-5D-5L). In some embodiments, the patient's quality of life is measured using the Kansas City Cardiomyopathy Questionnaire (KCCQ).

### Ejection Fraction

In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has an ejection fraction of less than 10% (e.g., less than 10, 15, 20, 25, 30, 35, 40, 45, 50, or 55%). In some embodiments, the method relates to patient's having an ejection fraction of less than 10%. In some embodiments, the method relates to patient's having an ejection fraction of less than 15%. In some embodiments, the method relates to patient's having an ejection fraction of less than 20%. In some embodiments, the method relates to patient's having an ejection fraction of less than 25%. In some embodiments, the method relates to patient's having an ejection fraction of less than 30%. In some embodiments, the method relates to patient's having an ejection fraction of less than 35%. In some embodiments, the method relates to patient's having an ejection fraction of less than 40%. In some embodiments, the method relates to patient's having an ejection fraction of less than 45%. In some embodiments, the method relates to patient's having an ejection fraction of less than 50%. In some embodiments, the method relates to patient's having an ejection fraction of less than 55%. In some embodiments, the ejection fraction is the right ventricular ejection fraction. In some embodiments, the ejection fraction is the left ventricular ejection fraction. In some embodiments, the ejection fraction is measured using an echocardiogram. In some embodiments, the patient has a preserved left ventricular ejection fraction.

In some embodiments, the disclosure relates to methods of adjusting one or more hemodynamic parameters in the PAH patient toward a more normal level (e.g., >50% ejection fraction), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to increasing the patient's ejection fraction by least 1%. In some embodiments, the method relates to increasing the patient's ejection fraction by at least 5%. In some embodiments, the method relates to increasing the patient's ejection fraction by at least 10%. In some embodiments, the method relates to increasing the patient's ejection fraction by at least 15%. In some embodiments, the method relates to increasing the patient's ejection fraction by at least 20%. In some embodiments, the method relates to increasing the patient's ejection fraction by at least 25%. In some embodiments, the method relates to increasing the patient's ejection fraction by at least 30%. In some embodiments, the method relates to increasing the patient's ejection fraction by at least 35%. In some embodiments, the method relates to increasing the patient's ejection fraction by at least 40%. In some embodiments, the method relates to increasing the patient's ejection fraction by at least 45%. In some embodiments, the method relates to increasing the patient's ejection fraction by at least 50%. In some embodiments, the method relates to increasing the patient's ejection fraction by at least 55%. In some embodiments, the method relates to increasing the patient's ejection fraction by at least 60%. In some embodiments, the method relates to increasing the patient's ejection fraction by at least 65%. In some embodiments, the method relates to increasing the patient's ejection fraction by at least 70%. In some embodiments, the method relates to increasing the patient's ejection fraction by at least 75%. In some embodiments, the method relates to increasing the patient's ejection fraction by at least 80%. In some embodiments, the method relates to increasing the patient's ejection fraction by at least 85%. In some embodiments, the method relates to increasing the patient's ejection fraction by at least 90%. In some embodiments, the method relates to increasing the patient's ejection fraction by at least 95%. In some embodiments, the method relates to increasing the patient's ejection fraction by at least 100%.

### Right Ventricular Function

In certain aspects, the disclosure relates to methods of improving or maintaining right ventricular function in PAH comprising administering to a patient in need thereof an effective amount of an ActRII polypeptide (e.g., an amino acid sequence that is at least 90% identical to an amino acid sequence corresponding to residues 30-110 of SEQ ID NO: 1). Improvement or maintenance of right ventricular function can be assessed by many echocardiographic measurements. One such quantitative approach to assess right ventricular function is the measurement of the tricuspid annular plane systolic excursion (TAPSE). The TAPSE estimates RV systolic function by measuring the level of systolic excursion of the lateral tricuspid valve annulus towards the apex. Other echocardiographic measurements that may be used to assess maintenance and/or improvements in right ventricular function include, but are not limited to, right ventricular fractional area change (RVFAC), right ventricular end-diastolic area (RVEDA), right ventricular end-systolic area (RVESA), right ventricular ejection fraction (RVEF), right ventricular-pulmonary artery (RV-PA) coupling, pulmonary arterial systolic pressure (PASP), tricuspid regurgitation velocity (TRV), and right ventricular hypertrophy.

### TAPSE

The tricuspid annular plane systolic excursion (TAPSE) can be obtained using echocardiography and represents a measure of RV longitudinal function. The TAPSE has previously been shown to have good correlations with parameters estimating RV global systolic function. A TAPSE <17 mm is highly suggestive of RV systolic dysfunction. In some embodiments, an improvement or maintenance of right ventricular function in a PAH patient is measured as an increase in TAPSE. In some embodiments, a PAH patient with an improvement or maintenance of right ventricular function has a TAPSE between 20 mm-28 mm. In some embodiments, a PAH patient with an improvement or maintenance of right ventricular function has a TAPSE of at least 20 mm. In some embodiments, a PAH patient with an improvement or maintenance of right ventricular function has a TAPSE of at least 22 mm. In some embodiments, a PAH patient with an improvement or maintenance of right ventricular function has a TAPSE of at least 24 mm. In some embodiments, a PAH patient with an improvement or maintenance of right ventricular function has a TAPSE of at least 26 mm. In some embodiments, a PAH patient with an improvement or maintenance of right ventricular function has a TAPSE of at least 28 mm. In some embodiments, the TAPSE is measured using echocardiography.

In some embodiments, a PAH patient with an improvement or maintenance of right ventricular function has a TAPSE between 16 mm-30 mm. In some embodiments, a PAH patient with an improvement or maintenance of right ventricular function has a TAPSE between 18 mm-28 mm. In some embodiments, a PAH patient with an improvement or maintenance of right ventricular function has a TAPSE of at least 18 mm. In some embodiments, the TAPSE is measured using echocardiography.

### PASP

In certain aspects, the disclosure relates to methods of treating, preventing, or reducing the progression rate and/or severity of pulmonary arterial hypertension (PAH) comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has a pulmonary arterial systolic pressure (PASP) of at least 30 mmHg (e.g., at least 30, 35, 40, 45, 50, 55, 60, 65, 70, 75, or 80 mmHg). In some embodiments, the method relates to patients having a PASP of at least 30 mmHg. In some embodiments, the method relates to patients having a PASP of at least 35 mmHg. In some embodiments, the method relates to patients having a PASP of at least 40 mmHg. In some embodiments, the method relates to patients having a PASP of at least 45 mmHg. In some embodiments, the method relates to patients having a PASP of at least 50 mmHg. In some embodiments, the method relates to patients having a PASP of at least 55 mmHg. In some embodiments, the method relates to patients having a PASP of at least 60 mmHg. In some embodiments, the method relates to patients having a PASP of at least 65 mmHg. In some embodiments, the method relates to patients having a PASP of at least 70 mmHg. In some embodiments, the method relates to patients having a PASP of at least 75 mmHg. In some embodiments, the method relates to patients having a PASP of at least 80 mmHg. In some embodiments, the PASP is a resting PASP. In some embodiments, the PASP is determined using the tricuspid regurgitation velocity (TRV) and right arterial (RA) pressure. In some embodiments, the PASP is determined using the following formula:

$$PASP = {TRV}^{2} \times 4 + RA pressure$$

TRV has been shown to correlate with PASP at rest and with exercise. The pressure gradient between the right ventricle and the right atrium can be calculated using the modified Bernoulli equation (Δp=4V²).

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to improving the pulmonary arterial systolic pressure (PASP) in the patient. In some embodiments, the method relates to reducing PASP. In some embodiments, the method relates to reducing the patient's PASP by at least 1 mmHg (e.g., at least 1, 2, 3, 5, 7, 10, 12, 15, 20, 25, 30, or 35 mmHg). In some embodiments, the method relates to reducing the patient's PASP by at least 2 mmHg. In some embodiments, the method relates to reducing the patient's PASP by at least 3 mmHg. In certain embodiments, the method relates to reducing the patient's PASP by at least 5 mmHg. In certain embodiments, the method relates to reducing the patient's PASP by at least 7 mmHg. In certain embodiments, the method relates to reducing the patient's PASP by at least 10 mmHg. In certain embodiments, the method relates to reducing the patient's PASP by at least 12 mmHg. In certain embodiments, the method relates to reducing the patient's PASP by at least 15 mmHg. In certain embodiments, the method relates to reducing the patient's PASP by at least 20 mmHg. In certain embodiments, the method relates to reducing the patient's PASP by at least 25 mmHg. In certain embodiments, the method relates to reducing the patient's PASP by at least 30 mmHg. In certain embodiments, the method relates to reducing the patient's PASP by at least 35 mmHg.

In some embodiments, the disclosure relates to methods of adjusting one or more hemodynamic parameters in the PH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to reducing the patient's PASP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to reducing the patient's PASP by at least 1%. In some embodiments, the method relates to reducing the patient's PASP by at least 5%. In some embodiments, the method relates to reducing the patient's PASP by at least 10%. In some embodiments, the method relates to reducing the patient's PASP by at least 15%. In some embodiments, the method relates to reducing the patient's PASP by at least 20%. In some embodiments, the method relates to reducing the patient's PASP by at least 25%. In some embodiments, the method relates to reducing the patient's PASP by at least 30%. In some embodiments, the method relates to reducing the patient's PASP by at least 35%. In some embodiments, the method relates to reducing the patient's PASP by at least 40%. In some embodiments, the method relates to reducing the patient's PASP by at least 45%. In some embodiments, the method relates to reducing the patient's PASP by at least 50%. In some embodiments, the method relates to reducing the patient's PASP by at least 55%. In some embodiments, the method relates to reducing the patient's PASP by at least 60%. In some embodiments, the method relates to reducing the patient's PASP by at least 65%. In some embodiments, the method relates to reducing the patient's PASP by at least 70%. In some embodiments, the method relates to reducing the patient's PASP by at least 75%. In some embodiments, the method relates to reducing the patient's PASP by at least 80%. In some embodiments, the method relates to reducing the patient's PASP by at least 85%. In some embodiments, the method relates to reducing the patient's PASP by at least 90%. In some embodiments, the method relates to reducing the patient's PASP by at least 95%. In some embodiments, the method relates to reducing the patient's PASP by at least 100%.

### RV-PA Coupling

Right ventricular dysfunction is a central feature of PAH and the main factor affecting prognosis. Energy transfer between ventricle contractility and arterial afterload is termed coupling. Energy transfer specifically between the right ventricle (RV) and pulmonary artery is termed right ventricle-pulmonary artery (RV-PA) coupling. In some embodiments, right ventricular dysfunction is due to a decrease in RV-PA coupling. RV-PA coupling can be estimated non-invasively as a ratio of TAPSE/PASP values. In some embodiments, a TAPSE/PASP ratio of >0.31 mm/mm Hg may be associated with a better prognosis and reduced risk of clinical worsening. In some embodiments, the improvement in RV-PA coupling is due to an improvement in PASP. In some embodiments, the calculation of RV-PA coupling is dependent upon paired results for three parameters (e.g., TRV, RAP, and TAPSE).

In certain aspects, the disclosure relates to methods of treating, preventing, or reducing the progression rate and/or severity of pulmonary arterial hypertension (PAH) comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has a TAPSE/PASP ratio less than 0.31 mm/mmHg (e.g., less than 0.3, 0.25, 0.2, 0.15, or 0.1 mm/mmHg). In some embodiments, the method relates to patients having a TAPSE/PASP ratio less than 0.31 mm/mmHg. In some embodiments, the method relates to patients having a TAPSE/PASP ratio less than 0.3 mm/mmHg. In some embodiments, the method relates to patients having a TAPSE/PASP ratio less than 0.25 mm/mmHg. In some embodiments, the method relates to patients having a TAPSE/PASP ratio less than 0.2 mm/mmHg. In some embodiments, the method relates to patients having a TAPSE/PASP ratio less than 0.15 mm/mmHg. In some embodiments, the method relates to patients having a TAPSE/PASP ratio less than 0.1 mm/mmHg. In some embodiments, the method relates to patients having a decreased TAPSE/PASP ratio as compared to a normal TAPSE/PASP ratio.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to improving or maintaining the right ventricular function in the patient. In some embodiments, a PAH patient with an improvement or maintenance of right ventricular function has a TAPSE/PASP ratio greater than 0.3 mm/mmHg (e.g., greater than 0.31, 0.32, 0.33, 0.34, or 0.35 mm/mmHg). In some embodiments, a PAH patient with an improvement or maintenance of right ventricular function has a TAPSE/PASP ratio greater than 0.31 mm/mmHg. In some embodiments, a PAH patient with an improvement or maintenance of right ventricular function has a TAPSE/PASP ratio greater than 0.32 mm/mmHg. In some embodiments, a PAH patient with an improvement or maintenance of right ventricular function has a TAPSE/PASP ratio greater than 0.33 mm/mmHg. In some embodiments, a PAH patient with an improvement or maintenance of right ventricular function has a TAPSE/PASP ratio greater than 0.34 mm/mmHg. In some embodiments, a PAH patient with an improvement or maintenance of right ventricular function has a TAPSE/PASP ratio greater than 0.35 mm/mmHg. In some embodiments, the improvement in right ventricular function is an increase in TAPSE/PASP ratio. In some embodiments, the method relates to increasing the TAPSE/PASP ratio. In some embodiments, the method relates to increasing the patient's TAPSE/PASP ratio by at least 0.05 mm/mmHg. In some embodiments, the method relates to increasing the patient's TAPSE/PASP ratio by at least 0.07 mm/mmHg. In some embodiments, the method relates to increasing the patient's TAPSE/PASP ratio by at least 0.10 mm/mmHg. In some embodiments, the method relates to increasing the patient's TAPSE/PASP ratio by at least 0.12 mm/mmHg. In some embodiments, the method relates to increasing the patient's TAPSE/PASP ratio by at least 0.15 mm/mmHg. In some embodiments, the method relates to increasing the patient's TAPSE/PASP ratio by at least 0.18 mm/mmHg. In some embodiments, the method relates to increasing the patient's TAPSE/PASP ratio by at least 0.20 mm/mmHg.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to increasing the patient's TAPSE/PASP ratio by at least 1% (e.g., at least 1, 5, 10, 15, 20, 25, 30, 35, 40, 45, 50, 55, 60, 65, 70, 75, 80, 85, 90, 95, or 100%). In some embodiments, the method relates to increasing the patient's TAPSE/PASP ratio by at least 5%. In some embodiments, the method relates to increasing the patient's TAPSE/PASP ratio by at least 10%. In some embodiments, the method relates to increasing the patient's TAPSE/PASP ratio by at least 15%. In some embodiments, the method relates to increasing the patient's TAPSE/PASP ratio by at least 20%. In some embodiments, the method relates to increasing the patient's TAPSE/PASP ratio by at least 25%. In some embodiments, the method relates to increasing the patient's TAPSE/PASP ratio by at least 30%. In some embodiments, the method relates to increasing the patient's TAPSE/PASP ratio by at least 35%. In some embodiments, the method relates to increasing the patient's TAPSE/PASP ratio by at least 40%. In some embodiments, the method relates to increasing the patient's TAPSE/PASP ratio by at least 45%. In some embodiments, the method relates to increasing the patient's TAPSE/PASP ratio by at least 50%. In some embodiments, the method relates to increasing the patient's TAPSE/PASP ratio by at least 55%. In some embodiments, the method relates to increasing the patient's TAPSE/PASP ratio by at least 60%. In some embodiments, the method relates to increasing the patient's TAPSE/PASP ratio by at least 65%. In some embodiments, the method relates to increasing the patient's TAPSE/PASP ratio by at least 70%. In some embodiments, the method relates to increasing the patient's TAPSE/PASP ratio by at least 75%. In some embodiments, the method relates to increasing the patient's TAPSE/PASP ratio by at least 80%. In some embodiments, the method relates to increasing the patient's TAPSE/PASP ratio by at least 85%. In some embodiments, the method relates to increasing the patient's TAPSE/PASP ratio by at least 90%. In some embodiments, the method relates to increasing the patient's TAPSE/PASP ratio by at least 95%. In some embodiments, the method relates to increasing the patient's TAPSE/PASP ratio by at least 100%.

### RVFAC, RVEDA, and RVESA

Right ventricular fractional area change (RVFAC) is a non-invasive quantitative measure of right ventricular function. RVFAC can be calculated using the formula [(RVEDA-RVESA)/RVEDA]*100. In some embodiments, the RVFAC is measured using echocardiography. In some embodiments, normal RVFAC is approximately 47.5±8.6% in men and approximately 50.9±8.0% in women. See, e.g., Kou S, et al. European Heart Journal—Cardiovascular Imaging. 2014 Jun. 1; 15(6):680-90. In some embodiments, PAH patients have a decrease in RVFAC.

In certain aspects, the disclosure relates to methods of treating, preventing, or reducing the progression rate and/or severity of pulmonary arterial hypertension (PAH) comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has a RVFAC of less than 20% (e.g., less than 20, 25, 30, 35, or 40%). In some embodiments, the method relates to patients having a RVFAC of less than 25%. In some embodiments, the method relates to patients having a RVFAC of less than 30%. In some embodiments, the method relates to patients having a RVFAC of less than 35%. In some embodiments, the method relates to patients having a RVFAC of less than 40%.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to improving or maintaining the right ventricular function in the patient. In some embodiments, the improvement or maintenance of right ventricular function is due to an increase in right ventricular fractional area change (RVFAC). In some embodiments, a PAH patient with an improvement or maintenance of right ventricular function has a RVFAC between 32-56%. In some embodiments, a PAH patient with an improvement or maintenance of right ventricular function has a RVFAC of at least 32%. In some embodiments, a PAH patient with an improvement or maintenance of right ventricular function has a RVFAC of at least 34%. In some embodiments, a PAH patient with an improvement or maintenance of right ventricular function has a RVFAC of at least 35%. In some embodiments, a PAH patient with an improvement or maintenance of right ventricular function has a RVFAC of at least 36%. In some embodiments, a PAH patient with an improvement or maintenance of right ventricular function has a RVFAC of at least 38%. In some embodiments, a PAH patient with an improvement or maintenance of right ventricular function has a RVFAC of at least 40%. In some embodiments, a PAH patient with an improvement or maintenance of right ventricular function has a RVFAC of at least 42%. In some embodiments, a PAH patient with an improvement or maintenance of right ventricular function has a RVFAC of at least 44%. In some embodiments, a PAH patient with an improvement or maintenance of right ventricular function has a RVFAC of at least 46%. In some embodiments, a PAH patient with an improvement or maintenance of right ventricular function has a RVFAC of at least 48%. In some embodiments, a PAH patient with an improvement or maintenance of right ventricular function has a RVFAC of at least 50%. In some embodiments, a PAH patient with an improvement or maintenance of right ventricular function has a RVFAC of at least 52%. In some embodiments, a PAH patient with an improvement or maintenance of right ventricular function has a RVFAC of at least 54%. In some embodiments, a PAH patient with an improvement or maintenance of right ventricular function has a RVFAC of at least 56%.

In some embodiments, the disclosure relates to methods of adjusting one or more echocardiogram parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to decreasing the patient's RVEDA by least 1% (e.g., at least 1, 2, 3, 4, 5, 6, 7, 8, 9, 10, 12, 14, 16, 18, or 20%). In some embodiments, the method relates to increasing the patient's RVFAC by least 2%. In some embodiments, the method relates to increasing the patient's RVFAC by least 3%. In some embodiments, the method relates to increasing the patient's RVFAC by least 4%. In some embodiments, the method relates to increasing the patient's RVFAC by least 5%. In some embodiments, the method relates to increasing the patient's RVFAC by least 6%. In some embodiments, the method relates to increasing the patient's RVFAC by least 7%. In some embodiments, the method relates to increasing the patient's RVFAC by least 8%. In some embodiments, the method relates to increasing the patient's RVFAC by least 9%. In some embodiments, the method relates to increasing the patient's RVFAC by least 10%. In some embodiments, the method relates to increasing the patient's RVFAC by least 12%. In some embodiments, the method relates to increasing the patient's RVFAC by least 14%. In some embodiments, the method relates to increasing the patient's RVFAC by least 16%. In some embodiments, the method relates to increasing the patient's RVFAC by least 18%. In some embodiments, the method relates to increasing the patient's RVFAC by least 20%.

In some embodiments, the improvement in right ventricular function is due to an increase in ejection fraction. In some embodiments, the improvement in right ventricular function is due to an increase in ejection fraction and an increase in the patient's RVFAC.

The right ventricular end-diastolic area (RVEDA) can be measured using echocardiography. Nonnal RVEDA is approximately 18.2±4.3 cm² in men and approximately 14.8±3.5 cm² in women. See, e.g., Kou S, et al. European Heart Journal—Cardiovascular Imaging. 2014 Jun. 1; 15(6):680-90.

In certain aspects, the disclosure relates to methods of treating, preventing, or reducing the progression rate and/or severity of pulmonary arterial hypertension (PAH) comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has a RVEDA of at least 22 cm² (e.g., at least 22, 24, 26, 28, 30, 32, or 34 cm²). In some embodiments, the method relates to patients having a RVEDA of at least 24 cm². In some embodiments, the method relates to patients having a RVEDA of at least 26 cm². In some embodiments, the method relates to patients having a RVEDA of at least 28 cm². In some embodiments, the method relates to patients having a RVEDA of at least 30 cm². In some embodiments, the method relates to patients having a RVEDA of at least 32 cm². In some embodiments, the method relates to patients having a RVEDA of at least 34 cm². In some embodiments, the method relates to patients having increased RVEDA as compared to normal RVEDA.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to improving or maintaining the right ventricular function in the patient. In some embodiments, a PAH patient with an improvement or maintenance of right ventricular function has a RVEDA between 14-22 cm². In some embodiments, the improvement in right ventricular function is a reduction in RVEDA. In some embodiments, the method relates to reducing the RVEDA. In some embodiments, the method relates to reducing the patients RVEDA by at least 1 cm². In some embodiments, the method relates to reducing the patients RVEDA by at least 2 cm². In some embodiments, the method relates to reducing the patients RVEDA by at least 3 cm². In some embodiments, the method relates to reducing the patients RVEDA by at least 4 cm². In some embodiments, the method relates to reducing the patients RVEDA by at least 5 cm². In some embodiments, the method relates to reducing the patients RVEDA by at least 6 cm². In some embodiments, the method relates to reducing the patients RVEDA by at least 7 cm². In some embodiments, the method relates to reducing the patients RVEDA by at least 8 cm². In some embodiments, the method relates to reducing the patients RVEDA by at least 9 cm². In some embodiments, the method relates to reducing the patients RVEDA by at least 10 cm².

In some embodiments, the disclosure relates to methods of adjusting one or more echocardiogram parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to decreasing the patient's RVEDA by least 1% (e.g., at least 1, 5, 10, 15, 20, 25, 30, 35, or 40%). In some embodiments, the method relates to decreasing the patient's RVEDA by at least 5%. In some embodiments, the method relates to decreasing the patient's RVEDA by at least 10%. In some embodiments, the method relates to decreasing the patient's RVEDA by at least 15%. In some embodiments, the method relates to decreasing the patient's RVEDA by at least 20%. In some embodiments, the method relates to decreasing the patient's RVEDA by at least 25%. In some embodiments, the method relates to decreasing the patient's RVEDA by at least 30%. In some embodiments, the method relates to decreasing the patient's RVEDA by at least 35%. In some embodiments, the method relates to decreasing the patient's RVEDA by at least 40%.

The right ventricular end-systolic area (RVESA) can be measured using echocardiography. Normal RVESA is approximately 9.6±2.8 cm² in men and approximately 7.3±2.3 cm² in women. See, e.g., Kou S, et al. European Heart Journal—Cardiovascular Imaging. 2014 Jun. 1; 15(6):680-90.

In certain aspects, the disclosure relates to methods of treating, preventing, or reducing the progression rate and/or severity of pulmonary arterial hypertension (PAH) comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has a RVESA of at least 12 cm² (e.g., at least 12, 14, 16, 18, 20, 22, 24, 26, 28, 30, or 32 cm²). In some embodiments, the method relates to patients having a RVESA of at least 14 cm². In some embodiments, the method relates to patients having a RVESA of at least 16 cm². In some embodiments, the method relates to patients having a RVESA of at least 18 cm². In some embodiments, the method relates to patients having a RVESA of at least 20 cm². In some embodiments, the method relates to patients having a RVESA of at least 22 cm². In some embodiments, the method relates to patients having a RVESA of at least 24 cm². In some embodiments, the method relates to patients having a RVESA of at least 26 cm². In some embodiments, the method relates to patients having a RVESA of at least 28 cm². In some embodiments, the method relates to patients having a RVESA of at least 30 cm². In some embodiments, the method relates to patients having a RVESA of at least 32 cm². In some embodiments, the method relates to patients having increased RVESA as compared to normal RVESA.

In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to improving or maintaining the right ventricular function in the patient. In some embodiments, a PAH patient with an improvement or maintenance of right ventricular function has a RVESA of 7-20 cm². In some embodiments, the improvement in right ventricular function is a reduction in RVESA. In some embodiments, the method relates to reducing the RVESA. In some embodiments, the method relates to reducing the patient's RVESA by at least 1 cm². In some embodiments, the method relates to reducing the patient's RVESA by at least 2 cm². In some embodiments, the method relates to reducing the patient's RVESA by at least 3 cm². In some embodiments, the method relates to reducing the patient's RVESA by at least 4 cm². In some embodiments, the method relates to reducing the patient's RVESA by at least 5 cm². In some embodiments, the method relates to reducing the patient's RVESA by at least 6 cm². In some embodiments, the method relates to reducing the patient's RVESA by at least 7 cm². In some embodiments, the method relates to reducing the patient's RVESA by at least 8 cm². In some embodiments, the method relates to reducing the patient's RVESA by at least 9 cm². In some embodiments, the method relates to reducing the patient's RVESA by at least 10 cm².

In some embodiments, the disclosure relates to methods of adjusting one or more echocardiogram parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to decreasing the patient's RVESA by least 1% (e.g., at least 1, 2, 3, 4, 5, 10, 15, 20, 25, 30, 35, or 40%). In some embodiments, the method relates to decreasing the patient's RVESA by at least 2%. In some embodiments, the method relates to decreasing the patient's RVESA by at least 3%. In some embodiments, the method relates to decreasing the patient's RVESA by at least 4%. In some embodiments, the method relates to decreasing the patient's RVESA by at least 5%. In some embodiments, the method relates to decreasing the patient's RVESA by at least 10%. In some embodiments, the method relates to decreasing the patient's RVESA by at least 15%. In some embodiments, the method relates to decreasing the patient's RVESA by at least 20%. In some embodiments, the method relates to decreasing the patient's RVESA by at least 25%. In some embodiments, the method relates to decreasing the patient's RVESA by at least 30%. In some embodiments, the method relates to decreasing the patient's RVESA by at least 35%. In some embodiments, the method relates to decreasing the patient's RVESA by at least 40%.

### RVEF

Right ventricular ejection fraction is a global measure of RV systolic performance. RVEF can be calculated using the RV end-diastolic volume (RVEDV) and RV end systolic volume (RVESV). Specifically, RVEF can be calculated using the following formula: RVEF (%)=((RVEDV−RVESV)/RVEDV)*100. Normal RVEF is approximately 56-65% in men and 60-71% in women. See, e.g., Lang RM, J Am Soc Echocardiogr. 2015; 28(1):1-39.e14. In some embodiments, the RVEF is measured using echocardiography. In some embodiments, the disclosure relates to methods of adjusting one or more hemodynamic parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to improving or maintaining the right ventricular function in the patient. In some embodiments, a PAH patient with an improvement or maintenance of right ventricular function has a RVEF of 45-71%. In some embodiments, a PAH patient with an improvement or maintenance of right ventricular function has a RVEF of 45%. In some embodiments, a PAH patient with an improvement or maintenance of right ventricular function has a RVEF of 50%. In some embodiments, a PAH patient with an improvement or maintenance of right ventricular function has a RVEF of 55%. In some embodiments, a PAH patient with an improvement or maintenance of right ventricular function has a RVEF of 60%. In some embodiments, a PAH patient with an improvement or maintenance of right ventricular function has a RVEF of 65%. In some embodiments, a PAH patient with an improvement or maintenance of right ventricular function has a RVEF of 70%.

In some embodiments, the disclosure relates to methods of adjusting one or more echocardiogram parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to increasing the patient's RVEF by least 2%. In some embodiments, the method relates to increasing the patient's RVEF by least 3%. In some embodiments, the method relates to increasing the patient's RVEF by least 4%. In some embodiments, the method relates to increasing the patient's RVEF by least 5%. In some embodiments, the method relates to increasing the patient's RVEF by least 6%. In some embodiments, the method relates to increasing the patient's RVEF by least 7%. In some embodiments, the method relates to increasing the patient's RVEF by least 8%. In some embodiments, the method relates to increasing the patient's RVEF by least 9%. In some embodiments, the method relates to increasing the patient's RVEF by least 10%. In some embodiments, the method relates to increasing the patient's RVEF by least 11%. In some embodiments, the method relates to increasing the patient's RVEF by least 12%. In some embodiments, the method relates to increasing the patient's RVEF by least 13%. In some embodiments, the method relates to increasing the patient's RVEF by least 14%. In some embodiments, the method relates to increasing the patient's RVEF by least 15%. In some embodiments, the method relates to increasing the patient's RVEF to a normal value (e.g., between 56-65% in men and 60-71% in women).

### Right Ventricular Hypertrophy

In certain aspects, the improvement in right ventricular function is measured as a decrease in right ventricular hypertrophy. In some embodiments, the right ventricular hypertrophy is measured using the Fulton index (RV/(LV+S)).

In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has right ventricular hypertrophy. In some embodiments, the disclosure relates to methods of adjusting one or more parameters in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to decreasing right ventricular hypertrophy in the patient. In some embodiments, the method relates to decreasing the patient's right ventricular hypertrophy by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's right ventricular hypertrophy by at least 1%. In some embodiments, the method relates to decreasing the patient's right ventricular hypertrophy by at least 5%. In some embodiments, the method relates to decreasing the patient's right ventricular hypertrophy by at least 10%. In some embodiments, the method relates to decreasing the patient's right ventricular hypertrophy by at least 15%. In some embodiments, the method relates to decreasing the patient's right ventricular hypertrophy by at least 20%. In some embodiments, the method relates to decreasing the patient's right ventricular hypertrophy by at least 25%. In some embodiments, the method relates to decreasing the patient's right ventricular hypertrophy by at least 30%. In some embodiments, the method relates to decreasing the patient's right ventricular hypertrophy by at least 35%. In some embodiments, the method relates to decreasing the patient's right ventricular hypertrophy by at least 40%. In some embodiments, the method relates to decreasing the patient's right ventricular hypertrophy by at least 45%. In some embodiments, the method relates to decreasing the patient's right ventricular hypertrophy by at least 50%. In some embodiments, the method relates to decreasing the patient's right ventricular hypertrophy by at least 55%. In some embodiments, the method relates to decreasing the patient's right ventricular hypertrophy by at least 60%. In some embodiments, the method relates to decreasing the patient's right ventricular hypertrophy by at least 65%. In some embodiments, the method relates to decreasing the patient's right ventricular hypertrophy by at least 70%. In some embodiments, the method relates to decreasing the patient's right ventricular hypertrophy by at least 75%. In some embodiments, the method relates to decreasing the patient's right ventricular hypertrophy by at least 80%. In some embodiments, the method relates to decreasing the patient's right ventricular hypertrophy by at least 85%. In some embodiments, the method relates to decreasing the patient's right ventricular hypertrophy by at least 90%. In some embodiments, the method relates to decreasing the patient's right ventricular hypertrophy by at least 95%. In some embodiments, the method relates to decreasing the patient's right ventricular hypertrophy by at least 100%.

### Cardiac Output

Cardiac output is the volume of blood the heart pumps per minute. Cardiac output is calculated by multiplying the stroke volume by the heart rate. In general, normal cardiac output at rest is about 4 to 8 L/min. The cardiac index is an assessment of the cardiac output value based on the patient's size. To find the cardiac index, the cardiac output is divided by the person's body surface area (BSA). The normal range for CI is 2.5 to 4 L/min/m². Cardiac can decline by almost 40% without deviating from the normal limits. A low cardiac index of less than about 2.5 L/min/m² usually indicates a disturbance in cardiovascular performance. The cardiac output can be utilized to calculate the cardiac index (e.g., cardiac index=cardiac output/body surface area). The cardiac output can be also utilized to calculate the stroke volume (e.g., stroke volume=CO/heart rate). In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the method increases the patient's cardiac output by at least 5% (e.g., at least 5, 10, 15, 20, 25, 30, 35, 40, 45, 50, 55, 60, 65, 70, 75, 80, 85, 90, 95, or 100%). In some embodiments, the method relates to increasing the patient's cardiac output by at least 5%. In some embodiments, the method relates to increasing the patient's cardiac output by at least 10%. In some embodiments, the method relates to increasing the patient's cardiac output by at least 15%. In some embodiments, the method relates to increasing the patient's cardiac output by at least 20%. In some embodiments, the method relates to increasing the patient's cardiac output by at least 25%. In some embodiments, the method relates to increasing the patient's cardiac output by at least 30%. In some embodiments, the method relates to increasing the patient's cardiac output by at least 35%. In some embodiments, the method relates to increasing the patient's cardiac output by at least 40%. In some embodiments, the method relates to increasing the patient's cardiac output by at least 45%. In some embodiments, the method relates to increasing the patient's cardiac output by at least 50%. In some embodiments, the method relates to increasing the patient's cardiac output by at least 55%. In some embodiments, the method relates to increasing the patient's cardiac output by at least 60%. In some embodiments, the method relates to increasing the patient's cardiac output by at least 65%. In some embodiments, the method relates to increasing the patient's cardiac output by at least 70%. In some embodiments, the method relates to increasing the patient's cardiac output by at least 75%. In some embodiments, the method relates to increasing the patient's cardiac output by at least 80%. In some embodiments, the method relates to increasing the patient's cardiac output by at least 85%. In some embodiments, the method relates to increasing the patient's cardiac output by at least 90%. In some embodiments, the method relates to increasing the patient's cardiac output by at least 95%. In some embodiments, the method relates to increasing the patient's cardiac output by at least 100%. In some embodiments, the method relates to increasing the patient's cardiac index to at least 4.2 L/min/m². In some embodiments, the cardiac index is measured at rest. In some embodiments, the method relates to increasing the patient's cardiac output to at least 4 L/min. In some embodiments, the cardiac output is measured at rest. In some embodiments, the cardiac output is measured using a right heart catheter. In some embodiments, cardiac output is measured by thermodilution. In some embodiments, cardiac output is measured using the Fick method.

### Exercise Capacity (6 MWD and BDI)

In certain aspects, the disclosure relates to methods of increasing exercise capacity in a patient having PAH comprising administering to a patient in need thereof an effective amount of an ActRII polypeptide (e.g., an amino acid sequence that is at least 90% identical to an amino acid sequence corresponding to residues 30-110 of SEQ ID NO: 1). Any suitable measure of exercise capacity can be used. For example, exercise capacity in a 6-minute walk test (6MWT), which measures how far the subject can walk in 6 minutes, i.e., the 6-minute walk distance (6 MWD), is frequently used to assess pulmonary hypertension severity and disease progression. In certain aspects, the Borg dyspnea index (BDI) may be used to measure exercise capacity. The BDI is a numerical scale for assessing perceived dyspnea (breathing discomfort). It measures the degree of breathlessness, for example, after completion of the 6MWT, where a BDI of 0 indicates no breathlessness and 10 indicates maximum breathlessness. In some embodiments, the BDI is measured using the BORG CR10 scale.

In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has a 6 MWD of less than 550 meters (e.g., a 6 MWD of less than 550, 500, 450, 440, 400, 380, 350, 300, 250, 200, or 150 meters). In some embodiments, the method relates to patient's having a 6 MWD of between 150 to 550 meters. In some embodiments, the method relates to patient's having a 6 MWD of between 100 to 500 meters. In some embodiments, the method relates to patient's having a 6 MWD of between 150 to 500 meters. In some embodiments, the method relates to patient's having a 6 MWD of at least 100 meters. In some embodiments, the method relates to patient's having a 6 MWD of at least 150 meters. In some embodiments, the method relates to patient's having a 6 MWD of less than 550 meters. In some embodiments, the method relates to patient's having a 6 MWD of less than 500 meters. In some embodiments, the method relates to patient's having a 6 MWD of less than 450 meters. In some embodiments, the method relates to patient's having a 6 MWD of less than 440 meters. In some embodiments, the method relates to patient's having a 6 MWD of less than 400 meters. In some embodiments, the method relates to patient's having a 6 MWD of less than 380 meters. In some embodiments, the method relates to patient's having a 6 MWD of less than 350 meters. In some embodiments, the method relates to patient's having a 6 MWD of less than 300 meters. In some embodiments, the method relates to patient's having a 6 MWD of less than 250 meters. In some embodiments, the method relates to patient's having a 6 MWD of less than 200 meters. In some embodiments, the method relates to patient's having a 6 MWD of less than 150 meters. In some embodiments, the method relates to increasing the patient's 6 MWD to >380 meters. In some embodiments, the method relates to increasing the patient's 6 MWD to >440 meters. In some embodiments, the method relates to increasing the patient's 6 MWD to >500 meters. See, e.g., Galie N., et al Euro Heart J. (2016) 37, 67-119.

In some embodiments, the disclosure relates to methods of adjusting one or more measurements of exercise capacity in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to increasing the patient's 6 MWD by at least 10 meters. In some embodiments, the method relates to increasing the patient's 6 MWD by at least 20 meters. In some embodiments, the method relates to increasing the patient's 6 MWD by at least 25 meters. In some embodiments, the method relates to increasing the patient's 6 MWD by at least 30 meters. In some embodiments, the method relates to increasing the patient's 6 MWD by at least 40 meters. In some embodiments, the method relates to increasing the patient's 6 MWD by at least 50 meters. In some embodiments, the method relates to increasing the patient's 6 MWD by at least 60 meters. In some embodiments, the method relates to increasing the patient's 6 MWD by at least 70 meters. In some embodiments, the method relates to increasing the patient's 6 MWD by at least 80 meters. In some embodiments, the method relates to increasing the patient's 6 MWD by at least 90 meters. In some embodiments, the method relates to increasing the patient's 6 MWD by at least 100 meters. In some embodiments, the method relates to increasing the patient's 6 MWD by at least 125 meters. In some embodiments, the method relates to increasing the patient's 6 MWD by at least 150 meters. In some embodiments, the method relates to increasing the patient's 6 MWD by at least 175 meters. In some embodiments, the method relates to increasing the patient's 6 MWD by at least 200 meters. In some embodiments, the method relates to increasing the patient's 6 MWD by at least 250 meters. In some embodiments, the method relates to increasing the patient's 6 MWD by at least 300 meters. In some embodiments, the method relates to increasing the patient's 6 MWD by at least 400 meters. In some embodiments, the 6 MWD is tested after the patient has received 4 weeks of treatment utilizing an ActRII polypeptide disclosed herein. In some embodiments, the 6 MWD is tested after the patient has received 8 weeks of treatment utilizing an ActRII polypeptide disclosed herein. In some embodiments, the 6 MWD is tested after the patient has received 12 weeks of treatment utilizing an ActRII polypeptide disclosed herein. In some embodiments, the 6 MWD is tested after the patient has received 16 weeks of treatment utilizing an ActRII polypeptide disclosed herein. In some embodiments, the 6 MWD is tested after the patient has received 20 weeks of treatment utilizing an ActRII polypeptide disclosed herein. In some embodiments, the 6 MWD is tested after the patient has received 22 weeks of treatment utilizing an ActRII polypeptide disclosed herein. In some embodiments, the 6 MWD is tested after the patient has received 24 weeks of treatment utilizing an ActRII polypeptide disclosed herein. In some embodiments, the 6 MWD is tested after the patient has received 26 weeks of treatment utilizing an ActRII polypeptide disclosed herein. In some embodiments, the 6 MWD is tested after the patient has received 28 weeks of treatment utilizing an ActRII polypeptide disclosed herein. In some embodiments, the 6 MWD is tested after the patient has received 48 weeks of treatment utilizing an ActRII polypeptide disclosed herein.

In some embodiments, the disclosure relates to methods of adjusting one or more measurements of exercise capacity (e.g., BDI) in the PAH patient toward a more normal level (e.g., normal as compared to healthy people of similar age and sex), comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to reducing the patient's BDI. In some embodiments, the method relates to lowering the patient's BDI by at least 0.5 index points. In some embodiments, the method relates to lowering the patient's BDI by at least 1 index points. In some embodiments, the method relates to lowering the patient's BDI by at least 1.5 index points. In some embodiments, the method relates to lowering the patient's BDI by at least 2 index points. In some embodiments, the method relates to lowering the patient's BDI by at least 2.5 index points. In some embodiments, the method relates to lowering the patient's BDI by at least 3 index points. In some embodiments, the method relates to lowering the patient's BDI by at least 3.5 index points. In some embodiments, the method relates to lowering the patient's BDI by at least 4 index points. In some embodiments, the method relates to lowering the patient's BDI by at least 4.5 index points. In some embodiments, the method relates to lowering the patient's BDI by at least 5 index points. In some embodiments, the method relates to lowering the patient's BDI by at least 5.5 index points. In some embodiments, the method relates to lowering the patient's BDI by at least 6 index points. In some embodiments, the method relates to lowering the patient's BDI by at least 6.5 index points. In some embodiments, the method relates to lowering the patient's BDI by at least 7 index points. In some embodiments, the method relates to lowering the patient's BDI by at least 7.5 index points. In some embodiments, the method relates to lowering the patient's BDI by at least 8 index points. In some embodiments, the method relates to lowering the patient's BDI by at least 8.5 index points. In some embodiments, the method relates to lowering the patient's BDI by at least 9 index points. In some embodiments, the method relates to lowering the patient's BDI by at least 9.5 index points. In some embodiments, the method relates to lowering the patient's BDI by at least 10 index points.

### Echocardiography

There are numerous clinical presentation factors, echocardiography features, and other features that could be indicative of PAH. In patients suspected of having PAH, an echocardiogram may be used to measure the chamber sizes, particularly of the right atrium and right ventricle area, the magnitude of tricuspid regurgitation, the left ventricle eccentricity index and right ventricle contractility. The right ventricle contractility can be determined using several variables, such as the right ventricle longitudinal systolic strain/strain rate and right ventricle fractional area change. Tei index, and tricuspid annular plane systolic excursion. See, e.g., Galic N., et al Euro Heart J. (2016) 37, 67-119.

In a patient that has symptoms of PAH, an echocardiogram may be performed to evaluate various parameters. For instance, in some embodiments, an echocardiogram may be utilized to measure the tricuspid annular plane systolic excursion (TAPSE). In some embodiments, an echocardiogram may be utilized to measure the pulmonary arterial systolic pressure (PASP). In some embodiments, an echocardiogram may be utilized to measure the tricuspid regurgitation velocity (TRV). In some embodiments, an echocardiogram may be utilized to measure the right ventricular fractional area change (RVFAC). In some embodiments, an echocardiogram may be utilized to measure the right ventricular end-systolic area (RVESA). In some embodiments, an echocardiogram may be utilized to measure the right ventricular end-diastolic area (RVEDA). In some embodiments, an echocardiogram may be utilized to measure the right ventricular ejection fraction (RVEF). In some embodiments, an echocardiogram may be utilized to measure the right ventricular stroke volume (RVSV). In some embodiments, an echocardiogram may be utilized to measure the left ventricular ejection fraction (LVEF).

### Complications of PAH

In certain aspects, the disclosure relates to methods of treating, preventing, or reducing the progression rate and/or severity of one or more complications of PAH comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method relates to treating, preventing, or reducing the progression rate and/or severity of cell proliferation in the pulmonary artery of a PAH patient. In some embodiments, the method relates to treating, preventing, or reducing the progression rate and/or severity of smooth muscle and/or endothelial cells proliferation in the pulmonary artery of a PAH patient. In some embodiments, the method relates to treating, preventing, or reducing the progression rate and/or severity of angiogenesis in the pulmonary artery of a PAH patient. In some embodiments, the method relates to increasing physical activity of a patient having PAH. In some embodiments, the method relates to treating, preventing, or reducing the progression rate and/or severity of dyspnea in a PAH patient. In some embodiments, the method relates to treating, preventing, or reducing the progression rate and/or severity of chest pain in a PAH patient. In some embodiments, the method relates to treating, preventing, or reducing the progression rate and/or severity of fatigue in a PAH patient. In some embodiments, the method relates to treating, preventing, or reducing the progression rate and/or severity of pulmonary fibrosis in a PAH patient. In some embodiments, the method relates to treating, preventing, or reducing the progression rate and/or severity of fibrosis in a PAH patient. In some embodiments, the method relates to treating, preventing, or reducing the progression rate and/or severity of pulmonary vascular remodeling in a PAH patient. In some embodiments, the method relates to treating, preventing, or reducing the progression rate and/or severity of cardiac remodeling in a PAH patient. In some embodiments, the method relates to treating, preventing, or reducing the progression rate and/or severity of right ventricular hypertrophy in a PAH patient. In some embodiments, the method relates to treating, preventing, or reducing the progression rate and/or severity of metabolic syndrome in a PAH patient.

### Complications or Comorbidities

In some embodiments, the disclosure contemplates methods of treating one or more complications of PAH (e.g., smooth muscle and/or endothelial cell proliferation in the pulmonary artery, angiogenesis in the pulmonary artery, dyspnea, chest pain, pulmonary vascular remodeling, cardiac remodeling, right ventricular hypertrophy, pulmonary fibrosis, need for lung and/or heart transplant, and need for atrial septostomy) comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure contemplates methods of preventing one or more complications of PAH comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure contemplates methods of reducing the progression rate of one or more complications of PAH comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure contemplates methods of reducing the severity of one or more complications of PAH comprising administering to a patient in need thereof an effective amount of an ActRII polypeptide (e.g., an amino acid sequence that is at least 90% identical to an amino acid sequence corresponding to residues 30-110 of SEQ ID NO: 1).

In some embodiments, the disclosure contemplates methods of treating one or more comorbidities of PAH (e.g., systemic hypertension, decreased renal function, diabetes mellitus, obesity, coronary artery disease (CAD), heart failure, and anemia) comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the method results in the improvement of one or more comorbidities of PAH (e.g., systemic hypertension, decreased renal function, diabetes mellitus, obesity, coronary artery disease (CAD), heart failure, and anemia). In some embodiments, the one or more comorbidities of PAH are improved indirectly (e.g., due to an improvement in the patient's PH).

In some embodiments, the disclosure contemplates methods of reducing the progression rate of PAH comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure contemplates methods of reducing the severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure contemplates method of reducing the need to initiate treatment with a known treatment for PAH comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure contemplates method of reducing the need to increase the dose of prostacyclin in a patient (e.g., increasing the dose by at least 10%) comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure contemplates a method of reducing the need for PAH-specific hospitalization comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, PAH-specific hospitalization is hospitalization of patient for at least 24 hours. In some embodiments, the disclosure contemplates a method of reducing the deterioration of PAH comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, deterioration of PAH comprises worsening in WHO functional class and/or a decrease of at least 15% in the 6 MWD of the patient.

In some embodiments, a patient receiving one or more ActRII polypeptides disclosed herein (e.g., an amino acid sequence that is at least 90% identical to an amino acid sequence corresponding to residues 30-110 of SEQ ID NO: 1) will require a lower dosage or termination of the one or more therapies of PAH being co-administered with the one or more ActRII polypeptides. For example, if a patient is receiving one or more ActRII polypeptides in combination with one or more therapies for PAH (e.g., prostacyclin), the patient may require a decreased dosage of the one or more therapies for PAH (e.g., prostacyclin) if the patient is exhibiting signs of overdose of the one or more therapies for PAH (e.g., prostacyclin). For instance, prostacyclin dilates the systemic circulation as well as the pulmonary circulation and unneeded vasodilation may be detrimental to the patient. Patients who overdose on prostacyclin typically show excessively high rest cardiac outputs. In some embodiments, the dose of the one or more therapies for PAH (e.g., prostacyclin) will be reduced based on repeat cardiac output and hemodynamic measurements until the patient reaches a rest cardiac index of less than 4 L/m/m². For example, if a patient being treated with one or more ActRII polypeptides and one or more therapies for PAH (e.g., prostacyclin) shows a symptom of overdose (e.g., excessively high rest cardiac output), then dosing with the one or more therapies for PAH may be reduced (e.g., in amount and/or frequency) or dosing with the one or more therapies for PAH may be terminated.

In some embodiments, the disclosure contemplates a method of reducing the necessary dose of one or more therapies for PAH in a patient (e.g., decreasing the patient's dose by at least 10%) comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the disclosure contemplates method of reducing the necessary dose of prostacyclin in a patient (e.g., decreasing the patient's dose by at least 10%) comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the necessary dose of one or more therapies for PAH in the patient receiving an effective amount of an ActRII polypeptide is reduced by at least 10% (e.g., at least 10%, 20%, 30%, 40%, 50%, 60%, 70%, 80%, 90%, or 100%). In some embodiments, the necessary dose of one or more therapies for PAH in the patient receiving an effective amount of an ActRII polypeptide is reduced by at least 20%. In some embodiments, the necessary dose of one or more therapies for PAH in the patient receiving an effective amount of an ActRII polypeptide is reduced by at least 30%. In some embodiments, the necessary dose of one or more therapies for PAH in the patient receiving an effective amount of an ActRII polypeptide is reduced by at least 40%. In some embodiments, the necessary dose of one or more therapies for PAH in the patient receiving an effective amount of an ActRII polypeptide is reduced by at least 50%. In some embodiments, the necessary dose of one or more therapies for PAH in the patient receiving an effective amount of an ActRH polypeptide is reduced by at least 60%. In some embodiments, the necessary dose of one or more therapies for PAH in the patient receiving an effective amount of an ActRII polypeptide is reduced by at least 70%. In some embodiments, the necessary dose of one or more therapies for PAH in the patient receiving an effective amount of an ActRII polypeptide is reduced by at least 80%. In some embodiments, the necessary dose of one or more therapies for PAH in the patient receiving an effective amount of an ActRII polypeptide is reduced by at least 90%. In some embodiments, the necessary dose of one or more therapies for PAH in the patient receiving an effective amount of an ActRII polypeptide is reduced by 100%.

In some embodiments, the one or more therapies for PAH are one or more therapies for PAH disclosed herein. In some embodiments, the one or more therapies for PAH is selected from the group consisting of: phosphodiesterase type 5 inhibitors, soluble guanylate cyclase stimulators, prostacyclin receptor agonist, and endothelin receptor antagonists. In some embodiments, the one or more therapies for PAH is selected from the group consisting of: bosentan, sildenafil, beraprost, macitentan, selexipag, epoprostenol, treprostinil, iloprost, ambrisentan, and tadalafil.

### Transplant Free Survival

Lung and/or heart transplantation is a surgical treatment option for patients with PAH, and is often recommended for patients who don't respond to less invasive therapies (e.g., vasodilator therapy). Generally, PAH patients who receive lung and/or heart transplantation have Functional Class III or Class IV pulmonary hypertension in accordance with the World Health Organization's functional classification system for pulmonary hypertension.

In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the method increases the patient's transplant free survival by at least 1% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the method relates to increasing the patient's transplant free survival by at least 1%. In some embodiments, the method relates to increasing the patient's transplant free survival by at least 2%. In some embodiments, the method relates to increasing the patient's transplant free survival by at least 3%. In some embodiments, the method relates to increasing the patient's transplant free survival by at least 4%. In some embodiments, the method relates to increasing the patient's transplant free survival by at least 5%. In some embodiments, the method relates to increasing the patient's transplant free survival by at least 10%. In some embodiments, the method relates to increasing the patient's transplant free survival by at least 15%. In some embodiments, the method relates to increasing the patient's transplant free survival by at least 20%. In some embodiments, the method relates to increasing the patient's transplant free survival by at least 25%. In some embodiments, the method relates to increasing the patient's transplant free survival by at least 30%. In some embodiments, the method relates to increasing the patient's transplant free survival by at least 35%. In some embodiments, the method relates to increasing the patient's transplant free survival by at least 40%. In some embodiments, the method relates to increasing the patient's transplant free survival by at least 45%. In some embodiments, the method relates to increasing the patient's transplant free survival by at least 50%. In some embodiments, the method relates to increasing the patient's transplant free survival by at least 55%. In some embodiments, the method relates to increasing the patient's transplant free survival by at least 60%. In some embodiments, the method relates to increasing the patient's transplant free survival by at least 65%. In some embodiments, the method relates to increasing the patient's transplant free survival by at least 70%. In some embodiments, the method relates to increasing the patient's transplant free survival by at least 75%. In some embodiments, the method relates to increasing the patient's transplant free survival by at least 80%. In some embodiments, the method relates to increasing the patient's transplant free survival by at least 85%. In some embodiments, the method relates to increasing the patient's transplant free survival by at least 90%. In some embodiments, the method relates to increasing the patient's transplant free survival by at least 95%. In some embodiments, the method relates to increasing the patient's transplant free survival by at least 100%. In some embodiments, the method relates to increasing the patient's transplant free survival as compared to controls over 1 year. In some embodiments, the method relates to increasing the patient's transplant free survival as compared to controls over 2 years. In some embodiments, the method relates to increasing the patient's transplant free survival as compared to controls over 3 years. In some embodiments, the method relates to increasing the patient's transplant free survival as compared to controls over 4 years. In some embodiments, the method relates to increasing the patient's transplant free survival as compared to controls over 5 years. In some embodiments, the method relates to increasing the patient's transplant free survival as compared to controls over 6 years. In some embodiments, the method relates to increasing the patient's transplant free survival as compared to controls over 7 years.

### Death

In certain aspects, the disclosure relates to methods of reducing the risk of death in patients with PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the method reduces the patient's risk of death by at least 1% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the method relates to reducing the patient's risk of death by at least 1%. In some embodiments, the method relates to reducing the patient's risk of death by at least 2%. In some embodiments, the method relates to reducing the patient's risk of death by at least 3%. In some embodiments, the method relates to reducing the patient's risk of death by at least 4%. In some embodiments, the method relates to reducing the patient's risk of death by at least 5%. In some embodiments, the method relates to reducing the patient's risk of death by at least 10%. In some embodiments, the method relates to reducing the patient's risk of death by at least 15%. In some embodiments, the method relates to reducing the patient's risk of death by at least 20%. In some embodiments, the method relates to reducing the patient's risk of death by at least 25%. In some embodiments, the method relates to reducing the patient's risk of death by at least 30%. In some embodiments, the method relates to reducing the patient's risk of death by at least 35%. In some embodiments, the method relates to reducing the patient's risk of death by at least 40%. In some embodiments, the method relates to reducing the patient's risk of death by at least 45%. In some embodiments, the method relates to reducing the patient's risk of death by at least 50%. In some embodiments, the method relates to reducing the patient's risk of death by at least 55%. In some embodiments, the method relates to reducing the patient's risk of death by at least 60%. In some embodiments, the method relates to reducing the patient's risk of death by at least 65%. In some embodiments, the method relates to reducing the patient's risk of death by at least 70%. In some embodiments, the method relates to reducing the patient's risk of death by at least 75%. In some embodiments, the method relates to reducing the patient's risk of death by at least 80%. In some embodiments, the method relates to reducing the patient's risk of death by at least 85%. In some embodiments, the method relates to reducing the patient's risk of death by at least 90%. In some embodiments, the method relates to reducing the patient's risk of death by at least 95%. In some embodiments, the method relates to reducing the patient's risk of death by at least 100%. In some embodiments, the method reduces the risk of hospitalization for one or more complications associated with PAH.

### Combination Therapies

Optionally, methods disclosed herein for treating, preventing, or reducing the progression rate and/or severity of PAH, particularly treating, preventing, or reducing the progression rate and/or severity of one or more complications of PAH, may further comprise administering to the patient one or more supportive therapies or additional active agents for treating PAH. For example, the patient also may be administered one or more supportive therapies or active agents selected from the group consisting of: nitrates, hydralazine, prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, darusentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat, vericiguat, and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Tm); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1->3)-beta-D-glucopyranosyl]oleanolic acid; 3-O-[beta-D-glucopyranosyl-(1->2)-beta-D-glucopyranosyl]oleanolic acid; 3-0-[beta-D-glucopyranosyl-(1->3)-beta-D-glucopyranosyl]oleanolic acid 28-O-beta-D-glucopyranosyl ester; 3-O-[beta-D-glucopyranosyl-(1->2)-beta-D-glucopyranosyl]oleanolic acid 28-O-beta-D-glucopyranosyl ester; 3-O-[a-L-rhamnopyranosyl-(1->3)-beta-D-glucuronopyranosyl]oleanolic acid; 3-O-[alpha-L-rhamnopyranosyl-(1->3)-beta-D-glucuronopyranosyl]oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1--3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate), lung and/or heart transplantation. In some embodiments, the methods described herein may further comprise administering to the patient parental prostacyclin. In some embodiments, the methods described herein may further comprise administering to the patient one additional supportive therapy or additional active agent (i.e., double therapy) for treating PAH. In some embodiments, the methods described herein may further comprise administering to the patient two additional supportive therapies or additional active agents (i.e., triple therapy) for treating PAH. In some embodiments, the methods described herein may further comprise administering to the patient three additional supportive therapies or additional active agents (i.e., quadruple therapy) for treating PAH.

In some embodiments, the methods described herein may further comprise administering to the patient an angiotensin antagonist (e.g., angiotensin receptor blocker, ARB). In some embodiments, a patient is further administered one or more ARBs selected from the group consisting of losartan, irbesartan, olmesartan, candesartan, valsartan, fimasartan, azilsartan, salprisartan, and telmisartan. In some embodiments, a patient is administered losartan. In some embodiments, a patient is administered irbesartan. In some embodiments, a patient is administered olmesartan. In some embodiments, a patient is administered candesartan. In some embodiments, a patient is administered valsartan. In some embodiments, a patient is administered fimasartan. In some embodiments, a patient is administered azilsartan. In some embodiments, a patient is administered salprisartan. In some embodiments, a patient is administered telmisartan.

In some embodiments, the methods described herein may further comprise administering to the patient one or more ACE inhibitors. In some embodiments, the one or more ACE inhibitors are selected from the group consisting of benazepril, captopril, enalapril, lisinopril, perindopril, ramipril (e.g., ramipen), trandolapril, and zofenopril. In some embodiments, a patient is administered benazepril. In some embodiments, a patient is administered captopril. In some embodiments, a patient is administered enalapril. In some embodiments, a patient is administered lisinopril. In some embodiments, a patient is administered perindopril. In some embodiments, a patient is administered ramipril. In some embodiments, a patient is administered trandolapril. In some embodiments, a patient is administered zofenopril. In some embodiments, the methods described herein may further comprise administering to the patient an ARB and an ACE inhibitor. In some embodiments, an alternative approach to angiotensin antagonism is to combine an ACE inhibitor and/or ARB with an aldosterone antagonist.

In some embodiments, the one or more supportive therapies or additional active agents for treating PAH are administered prior to administration of the ActRII polypeptide. In some embodiments, the one or more supportive therapies or additional active agents for treating PAH are administered in combination with the ActRII polypeptide. In some embodiments, the one or more supportive therapies or additional active agents for treating PAH are administered after the administration of the ActRII polypeptide. As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

### Functional Classes

PAH at baseline can be mild, moderate or severe, as measured for example by World Health Organization (WHO) functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system: Functional Class I: pulmonary hypertension without resulting limitation of physical activity; ordinary physical activity does not cause undue dyspnea or fatigue, chest pain or near syncope; Functional Class II: pulmonary hypertension resulting in slight limitation of physical activity; patient comfortable at rest; ordinary physical activity causes undue dyspnea or fatigue, chest pain or near syncope; Functional Class III: pulmonary hypertension resulting in marked limitation of physical activity; patient comfortable at rest; less than ordinary activity causes undue dyspnea or fatigue, chest pain or near syncope; Functional Class IV: pulmonary hypertension resulting in inability to carry out any physical activity without symptoms; patient manifests signs of right-heart failure; dyspnea and/or fatigue may be present even at rest; discomfort is increased by any physical activity.

In certain aspects, the disclosure relates to methods of treating, preventing, or reducing the progression rate and/or severity of PAH (e.g., treating, preventing, or reducing the progression rate and/or severity of one or more complications of PH in WHO Group 1) comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has Functional Class I, Functional Class II, Functional Class III, or Functional Class IV pulmonary hypertension as recognized by the WHO. In some embodiments, the patient has Functional Class II pulmonary hypertension as recognized by the WHO. In some embodiments, the patient has Functional Class III pulmonary hypertension as recognized by the WHO. In some embodiments, the patient has Functional Class IV pulmonary hypertension as recognized by the WHO. In some embodiments, the patient has Functional Class II or Class III pulmonary hypertension as recognized by the WHO. In some embodiments, the patient has Functional Class II, Class III, or Class IV pulmonary hypertension as recognized by the WHO. In some embodiments, the patient has Functional Class I, Class II, Class III, or Class IV pulmonary hypertension as recognized by the WHO. In some embodiments, the method delays clinical worsening of PAH. In some embodiments, the method delays clinical worsening of PAH in accordance with the WHO's functional classification system for pulmonary hypertension.

In some embodiments, the disclosure relates to methods of preventing or reducing pulmonary hypertension Functional Class progression comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the reduction in Functional Class progression is a delay in Functional Class progression. In some embodiments, the method relates to preventing or decreasing pulmonary hypertension functional class progression as recognized by the WHO. In some embodiments, the method relates to a patient that has Functional Class I pulmonary hypertension as recognized by the WHO. In some embodiments, the method relates to preventing or reducing patient progression from Functional Class I pulmonary hypertension to Functional Class II pulmonary hypertension as recognized by the WHO. In some embodiments, the method relates to a patient that has Functional Class II pulmonary hypertension as recognized by the WHO. In some embodiments, the method relates to preventing or reducing patient progression from Functional Class II pulmonary hypertension to Functional Class III pulmonary hypertension as recognized by the WHO. In some embodiments, the method relates to a patient that has Functional Class III pulmonary hypertension as recognized by the WHO. In some embodiments, the method relates to preventing or reducing patient progression from Functional Class III pulmonary hypertension to Functional Class IV pulmonary hypertension as recognized by the WHO.

In certain aspects, the disclosure relates to methods of promoting or increasing pulmonary hypertension Functional Class regression in a PAH patient comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has Functional Class I, Functional Class II, Functional Class III, or Functional Class IV pulmonary hypertension as recognized by the WHO. In some embodiments, the method relates to a patient that has Functional Class II pulmonary hypertension as recognized by the WHO. In some embodiments, the method relates to promoting patient regression from Functional Class II pulmonary hypertension to Functional Class Ipulmonary hypertension as recognized by the WHO. In some embodiments, the method relates to a patient that has Functional Class III pulmonary hypertension as recognized by the WHO. In some embodiments, the method relates to promoting patient regression from Functional Class III pulmonary hypertension to Functional Class II pulmonary hypertension as recognized by the WHO. In some embodiments, the method relates to promoting patient regression from Functional Class III pulmonary hypertension to Functional Class I pulmonary hypertension as recognized by the WHO. In some embodiments, the method relates to a patient that has Functional Class IV pulmonary hypertension as recognized by the WHO. In some embodiments, the method relates to promoting patient regression from Functional Class IV pulmonary hypertension to Functional Class III pulmonary hypertension as recognized by the WHO. In some embodiments, the method relates to promoting patient regression from Functional Class IV pulmonary hypertension to Functional Class II pulmonary hypertension as recognized by the WHO. In some embodiments, the method relates to promoting patient regression from Functional Class IV pulmonary hypertension to Functional Class I pulmonary hypertension as recognized by the WHO.

The New York Heart Association (NYHA) functional classification (Table 9) has been used to describe the severity of symptoms and exercise intolerance in patients with pulmonary hypertension. The NYHA functional classification system provides a rapid assessment of patients' functional status in everyday clinical practice and is a well-established means of predicting prognosis. The four functional classes recognized by the NYHA functional classification system are shown in Table 9.

TABLE 9
New York Heart Association (NYHA) functional classification
of pulmonary hypertension based on severity of symptoms and
physical activity

| | |
|---|---|
| Class I | No limitation of physical activity. Ordinary physical activity |
| | does not cause undue breathlessness, fatigue, or palpitations. |
| Class II | Slight limitation of physical activity. Comfortable at rest, but |
| | ordinary physical activity results in undue breathlessness, |
| | fatigue, or palpitations. |
| Class III | Marked limitation of physical activity. Comfortable at rest, but |
| | less than ordinary physical activity results in undue |
| | breathlessness, fatigue, or palpitations. |
| Class IV | Unable to carry on any physical activity without discomfort. |
| | Symptoms at rest can be present If any physical activity is |
| | undertaken, discomfort is increased. |

In certain aspects, the disclosure relates to methods of treating, preventing, or reducing the progression rate and/or severity of PAH (e.g., treating, preventing, or reducing the progression rate and/or severity of one or more complications of PH in WHO Group 1) comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has Functional Class I, Functional Class II, Functional Class III, or Functional Class IV pulmonary hypertension as recognized by the NYHA.

In some embodiments, the method relates to a patient that has Functional Class I pulmonary hypertension as recognized by the NYHA. In some embodiments, a patient with Functional Class I pulmonary hypertension as recognized by the NYHA has no limitation of physical activity. In some embodiments, a patient with Functional Class I pulmonary hypertension as recognized by the NYHA experiences physical activity that does not cause undue breathlessness, fatigue, and/or palpitations. In some embodiments, the method relates to a patient that has Functional Class II pulmonary hypertension as recognized by the NYHA. In some embodiments, a patient with Functional Class II pulmonary hypertension as recognized by the NYHA has slight limitation of physical activity. In some embodiments, a patient with Functional Class II pulmonary hypertension as recognized by the NYHA experiences ordinary physical activity resulting in undue breathlessness, fatigue, or palpitations. In some embodiments, the method relates to a patient that has Functional Class III pulmonary hypertension as recognized by the NYHA. In some embodiments, a patient with Functional Class III pulmonary hypertension as recognized by the NYHA has marked limitation of physical activity. In some embodiments, a patient with Functional Class III pulmonary hypertension as recognized by the NYHA experiences less than ordinary physical activity resulting in undue breathlessness, fatigue, or palpitations. In some embodiments, the method relates to a patient that has Functional Class IV pulmonary hypertension as recognized by the NYHA. In some embodiments, a patient with Functional Class IV pulmonary hypertension as recognized by the NYHA is unable to carry on any physical activity without discomfort. In some embodiments, a patient with Functional Class IV pulmonary hypertension as recognized by the NYHA experiences symptoms at rest, as well as when any physical activity is undertaken, discomfort is increased. In some embodiments, the method relates to patients having Functional Class II or Class III pulmonary hypertension as recognized by the NYHA. In some embodiments, the method relates to patients having Functional Class II, Class III, or Class IV pulmonary hypertension as recognized by the NYHA. In some embodiments, the method relates to patients having Functional Class I, Class II, Class III, or Class IV pulmonary hypertension as recognized by the NYHA. In some embodiments, the method delays clinical worsening of PAH. In some embodiments, the method delays clinical worsening of PAH in accordance with the NYHA's functional classification system for pulmonary hypertension.

In some embodiments, the disclosure relates to methods of preventing or reducing pulmonary hypertension Functional Class progression comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the reduction in Functional Class progression is a delay in Functional Class progression. In some embodiments, the method relates to preventing or decreasing pulmonary hypertension functional class progression as recognized by the NYHA. In some embodiments, the disclosure relates to methods of promoting or increasing pulmonary hypertension Functional Class regression in a PAH patient comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the patient has Functional Class I, Functional Class II, Functional Class III, or Functional Class IV pulmonary hypertension as recognized by the NYHA. In some embodiments, the method relates to preventing or delaying patient progression from Functional Class Ipulmonary hypertension to Functional Class II pulmonary hypertension as recognized by the NYHA. In some embodiments, the method relates to promoting patient regression from Functional Class II pulmonary hypertension to Functional Class I pulmonary hypertension as recognized by the NYHA. In some embodiments, the method relates to preventing or delaying patient progression from Functional Class II pulmonary hypertension to Functional Class III pulmonary hypertension as recognized by the NYHA. In some embodiments, the method relates to promoting patient regression from Functional Class III pulmonary hypertension to Functional Class II pulmonary hypertension as recognized by the NYHA. In some embodiments, the method relates to promoting patient regression from Functional Class III pulmonary hypertension to Functional Class I pulmonary hypertension as recognized by the NYHA. In some embodiments, the method relates to preventing or delaying patient progression from Functional Class III pulmonary hypertension to Functional Class IV pulmonary hypertension as recognized by the NYHA. In some embodiments, the method relates to promoting patient regression from Functional Class IV pulmonary hypertension to Functional Class III pulmonary hypertension as recognized by the NYHA. In some embodiments, the method relates to promoting patient regression from Functional Class IV pulmonary hypertension to Functional Class II pulmonary hypertension as recognized by the NYHA. In some embodiments, the method relates to promoting patient regression from Functional Class IV pulmonary hypertension to Functional Class I pulmonary hypertension as recognized by the NYHA.

In some embodiments, functional class regression is tested after the patient has received 4 weeks of treatment utilizing an ActRII polypeptide disclosed herein. In some embodiments, functional class regression is tested after the patient has received 8 weeks of treatment utilizing an ActRII polypeptide disclosed herein. In some embodiments, functional class regression is tested after the patient has received 12 weeks of treatment utilizing an ActRII polypeptide disclosed herein. In some embodiments, functional class regression is tested after the patient has received 16 weeks of treatment utilizing an ActRII polypeptide disclosed herein. In some embodiments, functional class regression is tested after the patient has received 20 weeks of treatment utilizing an ActRII polypeptide disclosed herein. In some embodiments, functional class regression is tested after the patient has received 22 weeks of treatment utilizing an ActRII polypeptide disclosed herein. In some embodiments, functional class regression is tested after the patient has received 24 weeks of treatment utilizing an ActRII polypeptide disclosed herein. In some embodiments, functional class regression is tested after the patient has received 26 weeks of treatment utilizing an ActRII polypeptide disclosed herein. In some embodiments, functional class regression is tested after the patient has received 28 weeks of treatment utilizing an ActRII polypeptide disclosed herein. In some embodiments, functional class regression is tested after the patient has received 48 weeks of treatment utilizing an ActRII polypeptide disclosed herein.

### Immune Cell Infiltrates

In some embodiments, patients with PAH show signs of chronic inflammation and maladaptive fibrosis. In some embodiments, histologic examinations of lung tissue from patients show the presence of immune cell infiltrates, composed of lymphocytes, macrophages, dendritic cells, and mast cells in pulmonary vascular lesions. The presence of these immune cell infiltrates suggests that PAH is, in part, an inflammatory disease.

In certain aspects, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the method decreases macrophage infiltration in the patient's lung by at least 10% (e.g., at least 10%, 20%, 30%, 40%, 50%, 60%, 70%, 80%, 90%, or 100%). In some embodiments, the method relates to decreasing macrophage infiltration in the patient's lung.

In some embodiments, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the method decreases lymphocyte infiltration in the patient's lung by at least 10% (e.g., at least 10%, 20%, 30%, 40%, 50%, 60%, 70%, 80%, 90%, or 100%). In some embodiments, the method relates to decreasing lymphocyte infiltration in the patient's lung.

In some embodiments, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the method decreases dendritic cell infiltration in the patient's lung by at least 10% (e.g., at least 10%, 20%, 30%, 40%, 50%, 60%, 70%, 80%, 90%, or 100%). In some embodiments, the method relates to decreasing dendritic cell infiltration in the patient's lung.

In some embodiments, the disclosure relates to methods of treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1), wherein the method decreases mast cell infiltration in the patient's lung by at least 10% (e.g., at least 10%, 20%, 30%, 40%, 50%, 60%, 70%, 80%, 90%, or 100%). In some embodiments, the method relates to decreasing mast cell infiltration in the patient's lung.

### Sustained Therapeutic Effect

In certain aspects, the disclosure relates to methods of treating, preventing, or reducing the progression rate and/or severity of PAH in a sustained manner comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the sustained manner comprises a persistent therapeutic effect following the reduction in administration of an ActRII polypeptide described herein. In some embodiments, the sustained manner comprises a persistent therapeutic effect following the withdrawal of administration of an ActRII polypeptide described herein. In some embodiments, the persistent therapeutic effect relates to maintaining functional or hematologic measurements over time. In some embodiments, the persistent therapeutic effect is measured as a sustained reduction in PVR. In some embodiments, the patient's PVR level does not increase for at least 1 week to at least 12 weeks following withdrawal of an ActRII polypeptide treatment described herein. In some embodiments, the patient's PVR level does not increase for at least 1 week following withdrawal of an ActRII polypeptide treatment described herein. In some embodiments, the patient's PVR level does not increase for at least 2 weeks following withdrawal of an ActRII polypeptide treatment described herein. In some embodiments, the patient's PVR level does not increase for at least 3 weeks following withdrawal of an ActRII polypeptide treatment described herein. In some embodiments, the patient's PVR level does not increase for at least 4 weeks following withdrawal of an ActRII polypeptide treatment described herein. In some embodiments, the patient's PVR level does not increase for at least 5 weeks following withdrawal of an ActRII polypeptide treatment described herein. In some embodiments, the patient's PVR level does not increase for at least 6 weeks following withdrawal of an ActRII polypeptide treatment described herein. In some embodiments, the patient's PVR level does not increase for at least 1 month to at least 6 months following withdrawal of an ActRII polypeptide treatment described herein.

In certain aspects, the disclosure relates to methods of treating or preventing cardiopulmonary remodeling associated with pulmonary arterial hypertension in a patient, comprising administering to a patient in need thereof an effective amount of an ActRII polypeptide (e.g., an amino acid sequence that is at least 90% identical to an amino acid sequence corresponding to residues 30-110 of SEQ ID NO: 1), wherein said method slows down cardiac remodeling and/or reverses cardiac remodeling. In some embodiments, the reversal is a sustained reversal. In some embodiments, the cardiac remodeling is ventricle remodeling. In some embodiments, the ventricle remodeling is left ventricular remodeling. In some embodiments, the ventricle remodeling is right ventricular remodeling. In some embodiments, the cardiac remodeling is ventricular dilation. In some embodiments, the method decreases interventricular septal end diastole. In some embodiments, the method decreases posterior wall end diastole.

In some embodiments, echocardiographic measurements may be used to assess the persistent therapeutic effect. In some embodiments, the echocardiographic measurements include, but are not limited to, RV fractional area change (RVFAC), sPAP, tricuspid annular systolic velocity (TASV), and Tei index. In some embodiments, a patient treated with an ActRII polypeptide disclosed herein shows a persistent therapeutic effect. In some embodiments, the persistent therapeutic effect results in decreased intrusion of the ventral wall into the left ventricle. In some embodiments, the persistent therapeutic effect results in an increase in right ventricular fractional area change (RVFAC).

### Known Treatments for PAH

There is no known cure for PAH; current methods of treatment focus on prolonging patient lifespan and enhancing patient quality of life. This is usually associated with good exercise capacity, good right ventricle function, and a low mortality risk (e.g., bring and/or keeping the patient in WHO Functional Class I or Functional Class II). Current methods of treatment of PAH may include administration of: vasodilators such as prostacyclin, epoprostenol, and sildenafil; endothelin receptor antagonists such as bosentan; calcium channel blockers such as amlodipine, diltiazem, and nifedipine; anticoagulants such as warfarin; and diuretics. Treatment of PAH has also been carried out using oxygen therapy, atrial septostomy, pulmonary thromboendarterectomy, and lung and/or heart transplantation. Each of these methods, however, suffers from one or multiple drawbacks which may include lack of effectiveness, serious side effects, low patient compliance, and high cost. In certain aspects, the method relate to treating, preventing, or reducing the progression rate and/or severity of PAH comprising administering to a patient in need thereof an effective amount of an ActRII polypeptide (e.g., an amino acid sequence that is at least 90% identical to an amino acid sequence corresponding to residues 30-110 of SEQ ID NO: 1) in combination with one or more additional active agents and/or supportive therapies for treating PAH (e.g., vasodilators such as prostacyclin, epoprostenol, and sildenafil; endothelin receptor antagonists such as bosentan; calcium channel blockers such as amlodipine, diltiazem, and nifedipine; anticoagulants such as warfarin; diuretics; oxygen therapy; atrial septostomy; pulmonary thromboendarterectomy; and lung and/or heart transplantation); bardoxolone methyl or a derivative thereof; oleanolic acid or derivative thereof.

### Measuring Hematologic Parameters in a Patient

In certain embodiments, the present disclosure provides methods for managing a patient that has been treated with, or is a candidate to be treated with, one or more one or more ActRII polypeptides of the disclosure (e.g., an amino acid sequence that is at least 90% identical to an amino acid sequence corresponding to residues 30-110 of SEQ ID NO: 1) by measuring one or more hematologic parameters in the patient. The hematologic parameters may be used to evaluate appropriate dosing for a patient who is a candidate to be treated with one or more ActRII polypeptides of the present disclosure, to monitor the hematologic parameters during treatment, to evaluate whether to adjust the dosage during treatment with one or more ActRII polypeptides of the disclosure, and/or to evaluate an appropriate maintenance dose of one or more ActRII polypeptides of the disclosure. If one or more of the hematologic parameters are outside the normal level, dosing with one or more ActRII polypeptides may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art recognized methods. In other embodiments, hematologic parameters such as white blood cell levels, platelet levels, and neutrophil levels may be measured using art recognized methods. Such parameters may be determined using a blood sample from a patient. Increases in red blood cell levels, hemoglobin levels, and/or hematocrit levels may cause increases in blood pressure. Decreases in white blood cell levels, platelet levels, and/or neutrophil levels may indicate a need to decrease, delay, or discontinue treatment of the administration of one or more ActRII polypeptides of the disclosure.

In one embodiment, if one or more hematologic parameters are outside the normal range or on the high side of normal in a patient who is a candidate to be treated with one or more ActRII polypeptides, then onset of administration of the one or more ActRII polypeptides of the disclosure may be delayed until the hematologic parameters have returned to a normal or acceptable level either naturally or via therapeutic intervention. For example, if a candidate patient is hypertensive or pre-hypertensive, then the patient may be treated with a blood pressure lowering agent in order to reduce the patient's blood pressure. Any blood pressure lowering agent appropriate for the individual patient's condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient's iron stores have returned to a normal or acceptable level. For patients having higher than normal red blood cell levels and/or hemoglobin levels (e.g., hemoglobin levels >16.0 g/dL or hemoglobin levels >18.0 g/dL), then administration of the one or more ActRII polypeptides of the disclosure may be delayed or reduced until the levels have returned to a normal or acceptable level. In some embodiments, a normal or acceptable level of hemoglobin includes patients with hemoglobin levels between 8-15 g/dl. In some embodiments, a normal or acceptable level of hemoglobin includes patients with hemoglobin levels of <18 g/dl. In some embodiments, a normal or acceptable level of hemoglobin increase over time includes patients whose hemoglobin levels increase less than 2 g/dL over the first period of time in treatment. In some embodiments, the first period of time is 3 weeks. For patients having lower than normal white blood cell counts (e.g., leukopenia; white blood cell count <3000/mm³ or <3.0×10⁹/L (Grade 2)), then administration of the one or more ActRII polypeptides of the disclosure may be delayed or reduced until the levels have returned to a normal or acceptable level. For patients having lower than normal white blood cell counts (e.g., leukopenia; white blood cell count <2000/mm³ or <2.0×10⁹/L (Grade 3)), then administration of the one or more ActRII polypeptides of the disclosure may be delayed or reduced until the levels have returned to a normal or acceptable level. For patients having lower than normal platelet counts (e.g., thrombocytopenia; platelet count <75,000/mm³ or <75.0×10⁹/L (Grade 2)), then administration of the one or more ActRII polypeptides of the disclosure may be delayed or reduced until the levels have returned to a normal or acceptable level. For patients having lower than normal platelet counts (e.g., thrombocytopenia; platelet count <50,000/mm³ or <50.0×10⁹/L (Grade 3)), then administration of the one or more ActRII polypeptides of the disclosure may be delayed or reduced until the levels have returned to a normal or acceptable level. For patients having lower than normal neutrophil counts (e.g., neutropenia; neutrophil count <1500/mm³ or <1.5×10⁹/L (Grade 2)), then administration of the one or more ActRII polypeptides of the disclosure may be delayed or reduced until the levels have returned to a normal or acceptable level. For patients having lower than normal neutrophil counts (e.g., neutropenia; neutrophil count <1000/mm³ or <1.0×10⁹/L (Grade 3)), then administration of the one or more ActRII polypeptides of the disclosure may be delayed or reduced until the levels have returned to a normal or acceptable level.

In certain embodiments, if one or more hematologic parameters are outside the normal range or on the high side of normal in a patient who is a candidate to be treated with one or more ActRII polypeptides, then the onset of administration may not be delayed. However, the dosage amount or frequency of dosing of the one or more ActRII polypeptides of the disclosure may be set at an amount that would reduce the risk of an unacceptable increase in the hematologic parameters arising upon administration of the one or more ActRII polypeptides of the disclosure. Alternatively, a therapeutic regimen may be developed for the patient that combines one or more ActRII polypeptides with a therapeutic agent that addresses the undesirable level of the hematologic parameter. For example, if the patient has elevated blood pressure, then a therapeutic regimen may be designed involving administration of one or more ActRII polypeptides and a blood pressure lowering agent. For a patient having lower than desired iron stores, a therapeutic regimen may be developed involving one or more ActRII polypeptides of the disclosure and iron supplementation.

In one embodiment, baseline parameter(s) for one or more hematologic parameters may be established for a patient who is a candidate to be treated with one or more ActRII polypeptides of the disclosure and an appropriate dosing regimen established for that patient based on the baseline value(s). Alternatively, established baseline parameters based on a patient's medical history could be used to inform an appropriate ActRII polypeptide dosing regimen for a patient. For example, if a healthy patient has an established baseline blood pressure reading that is above the defined normal range it may not be necessary to bring the patient's blood pressure into the range that is considered normal for the general population prior to treatment with the one or more ActRII polypeptides of the disclosure. A patient's baseline values for one or more hematologic parameters prior to treatment with one or more ActRII polypeptides of the disclosure may also be used as the relevant comparative values for monitoring any changes to the hematologic parameters during treatment with the one or more ActRII polypeptides of the disclosure.

In certain embodiments, one or more hematologic parameters are measured in patients who are being treated with one or more ActRII polypeptides. The hematologic parameters may be used to monitor the patient during treatment and permit adjustment or termination of the dosing with the one or more ActRII polypeptides of the disclosure or additional dosing with another therapeutic agent. For example, if administration of one or more ActRII polypeptides results in an increase in blood pressure, red blood cell level, or hemoglobin level, or a reduction in iron stores, white blood cell count, platelet count, or absolute neutrophil count, then the dose of the one or more ActRII polypeptides of the disclosure may be reduced in amount or frequency in order to decrease the effects of the one or more ActRII polypeptides of the disclosure on the one or more hematologic parameters. If administration of one or more ActRII polypeptides results in a change in one or more hematologic parameters that is adverse to the patient, then the dosing of the one or more ActRII polypeptides of the disclosure may be terminated either temporarily, until the hematologic parameter(s) return to an acceptable level, or permanently. Similarly, if one or more hematologic parameters are not brought within an acceptable range after reducing the dose or frequency of administration of the one or more ActRII polypeptides of the disclosure, then the dosing may be terminated. As an alternative, or in addition to, reducing or terminating the dosing with the one or more ActRII polypeptides of the disclosure, the patient may be dosed with an additional therapeutic agent that addresses the undesirable level in the hematologic parameter(s), such as, for example, a blood pressure lowering agent or an iron supplement. For example, if a patient being treated with one or more ActRII polypeptides has elevated blood pressure, then dosing with the one or more ActRII polypeptides of the disclosure may continue at the same level and a blood-pressure-lowering agent is added to the treatment regimen, dosing with the one or more antagonist of the disclosure may be reduced (e.g., in amount and/or frequency) and a blood-pressure-lowering agent is added to the treatment regimen, or dosing with the one or more antagonist of the disclosure may be terminated and the patient may be treated with a blood-pressure-lowering agent.

### Measuring Various Parameters Over Time

In certain embodiments, one or more of the measurements of pulmonary hypertension (e.g., pulmonary arterial hypertension) described herein can be measured over various periods of treatment time. In some embodiments, one or more of the measurements of pulmonary hypertension described herein is measured after the patient has received 4 weeks of treatment utilizing an ActRII polypeptide disclosed herein. In some embodiments, one or more of the measurements of pulmonary hypertension described herein is measured after the patient has received 8 weeks of treatment utilizing an ActRII polypeptide disclosed herein. In some embodiments, one or more of the measurements of pulmonary hypertension described herein is measured after the patient has received 12 weeks of treatment utilizing an ActRII polypeptide disclosed herein. In some embodiments, one or more of the measurements of pulmonary hypertension described herein is measured after the patient has received 16 weeks of treatment utilizing an ActRII polypeptide disclosed herein. In some embodiments, one or more of the measurements of pulmonary hypertension described herein is measured after the patient has received 20 weeks of treatment utilizing an ActRII polypeptide disclosed herein. In some embodiments, one or more of the measurements of pulmonary hypertension described herein is measured after the patient has received 22 weeks of treatment utilizing an ActRII polypeptide disclosed herein. In some embodiments, one or more of the measurements of pulmonary hypertension described herein is measured after the patient has received 24 weeks of treatment utilizing an ActRII polypeptide disclosed herein. In some embodiments, one or more of the measurements of pulmonary hypertension described herein is measured after the patient has received 26 weeks of treatment utilizing an ActRII polypeptide disclosed herein. In some embodiments, one or more of the measurements of pulmonary hypertension described herein is measured after the patient has received 28 weeks of treatment utilizing an ActRII polypeptide disclosed herein. In some embodiments, one or more of the measurements of pulmonary hypertension described herein is measured after the patient has received 48 weeks of treatment utilizing an ActRII polypeptide disclosed herein.

### 5. Pharmaceutical Compositions & Modes of Administration

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than the ActRII polypeptides which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more ActRII polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind described herein.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Further, the composition may be encapsulated or injected in a form for delivery to a target tissue site. In certain embodiments, compositions of the present invention may include a matrix capable of delivering one or more therapeutic compounds (e.g., ActRII polypeptides) to a target tissue site, providing a structure for the developing tissue and optimally capable of being resorbed into the body. For example, the matrix may provide slow release of the ActRII polypeptide. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments, methods of the invention can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., ActRII polypeptides). The various factors include, but are not limited to, the patient's age, sex, and diet, the severity disease, time of administration, and other clinical factors. Optionally, the dosage may vary with the type of matrix used in the reconstitution and the types of compounds in the composition. In some embodiments, a patient's hematologic parameters can be monitored by periodic assessments in order to determine if they have higher than normal red blood cell levels and/or hemoglobin levels (e.g., hemoglobin levels >16.0 g/dL or hemoglobin levels >18.0 g/dL). In some embodiments, a patient having higher than normal red blood cell levels and/or hemoglobin levels may receive a delayed or reduced dose until the levels have returned to a normal or acceptable level.

The probability of a patient having hemoglobin levels greater than 18 g/dL or increases in hemoglobin of greater than 2 g/dL may be higher during initial treatment with an ActRII polypeptide. In certain embodiments, a dosing regimen can be used to prevent, ameliorate, or decrease the adverse changes in hemoglobin levels. In some embodiments, ActRII polypeptides of the disclosure are administered using a dosing regimen. In some embodiments, the method comprises administering a dosing regimen of a therapeutically effective amount of an ActRII polypeptide as disclosed herein to a patient, comprising a first dose of between 0.1 mg/kg and 1.0 mg/kg of said polypeptide for a first period of time, and a second dose of between 0.1 mg/kg and 1.0 mg/kg of said polypeptide subsequently administered for a second period of time. In some embodiments, the method comprises administering a dosing regimen of therapeutically effective amount of an ActRII polypeptide as disclosed herein to a patient, comprising a first dose of between 0.1 mg/kg and 1.0 mg/kg of said polypeptide for a first period of time, a second dose of between 0.1 mg/kg and 1.0 mg/kg of said polypeptide administered for a second period of time, and a third dose of between 0.1 mg/kg and 1.0 mg/kg of said polypeptide subsequently administered for a third period of time. In some embodiments, the first dose of ActRII polypeptide is administered to a patient in an amount from about 0.2 mg/kg to about 0.4 mg/kg. In some embodiments, the first dose of ActRII polypeptide is administered to a patient at a dose of 0.3 mg/kg. In some embodiments, the second dose of ActRII polypeptide is administered to a patient in an amount from about 0.5 mg/kg to about 0.8 mg/kg. In some embodiments, the second dose of ActRII polypeptide is administered to a patient at a dose of 0.7 mg/kg. In some embodiments, the third dose of ActRII polypeptide is administered to a patient in an amount from about 0.2 mg/kg to about 0.4 mg/kg. In some embodiments, the third dose of ActRII polypeptide is administered to a patient at a dose of 0.3 mg/kg.

In some embodiments, the dosing regimen comprises administering a first dose of ActRII polypeptide to a patient in an amount of 0.3 mg/kg followed by administration of a second dose of ActRII polypeptide to the patient in an amount of 0.7 mg/kg. In some embodiments, the dosing regimen comprises administering a first dose of ActRII polypeptide to a patient in an amount of 0.3 mg/kg, administering a second dose of ActRII polypeptide to the patient in an amount of 0.7 mg/kg, and administering a third dose of ActRII polypeptide to the patient in an amount of 0.3 mg/kg. In some embodiments, the second dose exceeds the first dose. In some embodiments, the first dose exceeds the second dose. In some embodiments, the third dose exceeds the second dose. In some embodiments, the second dose exceeds the third dose. In some embodiments, the first period of time is at least 3 weeks. In some embodiments, the second period of time is at least 3 weeks. In some embodiments, the third period of time is at least 3 weeks. In some embodiments, the second period of time is at least 21 weeks. In some embodiments, the second period of time is at least 45 weeks. In some embodiments, the second period of time exceeds the first period of time. In some embodiments, the third period of time exceeds the first period of time. In some embodiments, the third period of time exceeds the second period of time.

In some embodiments, the change in dosing between the first dose and the second dose is determined by the attending physician considering various factors (e.g., hemoglobin levels). In some embodiments, the change in dosing between the second dose and the third dose is determined by the attending physician considering various factors (e.g., hemoglobin levels). In some embodiments, the various factors include, but are not limited to, the patient's change in hematologic parameters over a period of time. In some embodiments, a patient's hematologic parameters are monitored in order to determine if they have higher than normal red blood cell levels and/or hemoglobin levels (e.g., hemoglobin levels >16.0 g/dL or hemoglobin levels >18.0 g/dL). In some embodiments, a patient's hematologic parameters are monitored in order to determine if they have a higher than normal increase in hemoglobin levels over a period of time (e.g., hemoglobin level increase of >2 g/dL in less than 3 weeks). In some embodiments, the patient's dose of an ActRII polypeptide as disclosed herein will be decreased (e.g., decrease in dose from 0.7 mg/kg to 0.3 mg/kg) if one or more of the patient's hematologic parameters before or during treatment is abnormal. In some embodiments, the patient's dose of an ActRII polypeptide as disclosed herein will be maintained (e.g., maintained at 0.3 mg/kg or 0.7 mg/kg) if one or more of the patient's hematologic parameters before or during treatment is abnormal.

In some embodiments, the dosing regimen prevents, ameliorates, or decreases adverse effects of the ActRII polypeptide. In some embodiments, administration of an ActRII polypeptide in accordance with the dosage regimen as provided herein results in decreased adverse side effects. In some embodiments, administration of an ActRII polypeptide in accordance with the dosage regimen as provided herein decreases the probability of having hemoglobin levels greater than 18 g/dL during the first period of time. In some embodiments, administration of an ActRII polypeptide in accordance with the dosage regimen as provided herein decreases the probability of having hemoglobin levels greater than 18 g/dL in the first 3 weeks of treatment. In some embodiments, administration of an ActRII polypeptide in accordance with the dosage regimen as provided herein decreases the probability of increasing hemoglobin levels by greater than 2 g/dL during the first period of time. In some embodiments, administration of an ActRII polypeptide in accordance with the dosage regimen as provided herein decreases the probability of increasing hemoglobin levels by greater than 2 g/dL in the first 3 weeks of treatment.

In some embodiments, ActRII polypeptides of the disclosure are administered at a dosing range of 0.1 mg/kg to 2.0 mg/kg. In some embodiments, ActRII polypeptides of the disclosure are administered at 0.1 mg/kg. In some embodiments, ActRII polypeptides of the disclosure are administered at 0.2 mg/kg. In some embodiments, ActRII polypeptides of the disclosure are administered at 0.3 mg/kg. In some embodiments, ActRII polypeptides of the disclosure are administered at 0.4 mg/kg. In some embodiments, ActRII polypeptides of the disclosure are administered at 0.5 mg/kg. In some embodiments, ActRII polypeptides of the disclosure are administered at 0.6 mg/kg. In some embodiments, ActRII polypeptides of the disclosure are administered at 0.7 mg/kg. In some embodiments, ActRII polypeptides of the disclosure are administered at 0.8 mg/kg. In some embodiments, ActRII polypeptides of the disclosure are administered at 0.9 mg/kg. In some embodiments, ActRII polypeptides of the disclosure are administered at 1.0 mg/kg. In some embodiments, ActRII polypeptides of the disclosure are administered at 1.1 mg/kg. In some embodiments, ActRII polypeptides of the disclosure are administered at 1.2 mg/kg. In some embodiments, ActRII polypeptides of the disclosure are administered at 1.3 mg/kg. In some embodiments, ActRII polypeptides of the disclosure are administered at 1.4 mg/kg. In some embodiments, ActRII polypeptides of the disclosure are administered at 1.5 mg/kg. In some embodiments, ActRII polypeptides of the disclosure are administered at 1.6 mg/kg. In some embodiments, ActRII polypeptides of the disclosure are administered at 1.7 mg/kg. In some embodiments, ActRII polypeptides of the disclosure are administered at 1.8 mg/kg. In some embodiments, ActRII polypeptides of the disclosure are administered at 1.9 mg/kg. In some embodiments, ActRII polypeptides of the disclosure are administered at 2.0 mg/kg.

In certain embodiments, ActRII polypeptides of the disclosure are administered once a day. In certain embodiments, ActRII polypeptides of the disclosure are administered twice a day. In certain embodiments, ActRII polypeptides of the disclosure are administered once a week. In certain embodiments, ActRII polypeptides of the disclosure are administered twice a week. In certain embodiments. ActRII polypeptides of the disclosure are administered three times a week. In certain embodiments, ActRII polypeptides of the disclosure are administered every two weeks. In certain embodiments, ActRII polypeptides of the disclosure are administered every three weeks. In certain embodiments, ActRII polypeptides of the disclosure are administered every four weeks. In certain embodiments, ActRII polypeptides of the disclosure are administered every month.

In certain embodiments, the present invention also provides gene therapy for the in vivo production of ActRII polypeptides. Such therapy would achieve its therapeutic effect by introduction of the ActRII polypeptide polynucleotide sequences into cells or tissues having the disorders as listed above. Delivery of ActRII polypeptide polynucleotide sequences can be achieved using a recombinant expression vector such as a chimeric virus or a colloidal dispersion system. Preferred for therapeutic delivery of ActRII polypeptide polynucleotide sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the ActRII polypeptide. In a preferred embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for ActRII polypeptide polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### 6. Kits

The present disclosure provides a kit comprising a lyophilized polypeptide and an injection device. In certain embodiments, the lyophilized polypeptide comprises an ActRII polypeptide (e.g., a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1), or fragments, functional variants, or modified forms thereof. In certain embodiments, the lyophilized polypeptide is capable of binding to one or more ligands selected from the group consisting of activin A, activin B, and GDF11. In certain such embodiments, the lyophilized polypeptide is capable of binding to one or more ligands selected from the group consisting of BMP10, GDF8, and BMP6. In certain embodiments, the lyophilized polypeptide is capable of binding to activin and/or GDF11.

In some embodiments, the lyophilized polypeptide comprises a polypeptide that comprises, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of a polypeptide beginning at a residue corresponding to any one of amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 1. In certain such embodiments, the polypeptide comprises an amino acid sequence that is least 90%, 95%, or 99% identical to an amino acid sequence corresponding to residues 30-110 of SEQ ID NO: 1, wherein the polypeptide binds to activin and/or GDF11. In certain embodiments, the polypeptide comprises the amino acid sequence corresponding to residues 30-110 of SEQ ID NO: 1. In other embodiments, the polypeptide consists of the amino acid sequence corresponding to residues 30-110 of SEQ ID NO: 1. In certain embodiments, the polypeptide is a polypeptide comprising an amino acid sequence that is at least 90%, 95%, or 99% identical to the amino acid sequence corresponding to residues 21-135 of SEQ ID NO: 1. In certain embodiments, the polypeptide comprises the amino acid sequence corresponding to residues 21-135 of SEQ ID NO: 1. In other embodiments, the polypeptide consists of the amino acid sequence corresponding to residues 21-135 of SEQ ID NO: 1.

In some embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 2. In certain embodiments, the polypeptide consists essentially of the amino acid sequence of SEQ ID NO: 2. In other embodiments, the polypeptide consists of the amino acid sequence of SEQ ID NO: 2.

In some embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3. In certain embodiments, the polypeptide consists of the amino acid sequence of SEQ ID NO: 3. In other embodiments, the polypeptide consists of the amino acid sequence of SEQ ID NO: 3.

In certain embodiments of the foregoing, the lyophilized polypeptide comprises a fusion protein further comprising an Fc domain of an immunoglobulin. In certain such embodiments, the Fc domain of the immunoglobulin is an Fc domain of an IgG1 immunoglobulin. In other embodiments, the fusion protein further comprises a linker domain positioned between the polypeptide domain and the Fc domain of the immunoglobulin. In certain embodiments, the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21). In certain embodiments, the linker domain comprises TGGG (SEQ ID NO: 20).

In certain embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 23. In certain embodiments, the polypeptide consists of the amino acid sequence of SEQ ID NO: 23. In other embodiments, the polypeptide consists of the amino acid sequence of SEQ ID NO: 23.

In certain embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 30. In certain embodiments, the polypeptide consists of the amino acid sequence of SEQ ID NO: 30. In other embodiments, the polypeptide consists of the amino acid sequence of SEQ ID NO: 30.

In certain embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 41. In certain embodiments, the polypeptide consists of the amino acid sequence of SEQ ID NO: 41. In other embodiments, the polypeptide consists of the amino acid sequence of SEQ ID NO: 41.

In certain embodiments, the lyophilized polypeptide comprises one or more ActRII polypeptides as those disclosed herein. In certain embodiments, the lyophilized polypeptide comprises an ActRII polypeptide disclosed herein.

In certain embodiments, the lyophilized polypeptide is part of a homodimer protein complex. In certain embodiments, the polypeptide is glycosylated.

The present disclosure provides a kit comprising a sterile powder comprising a lyophilized polypeptide as disclosed herein and an injection device. In some embodiments of the kits disclosed herein, the sterile powder comprising a lyophilized polypeptide is pre-filled in one or more containers, such as one or more vials [FIG. 19 (1)].

In certain embodiments, the pH range for the sterile powder comprising a lyophilized polypeptide is from 7 to 8. In some embodiments, the sterile powder comprising a lyophilized polypeptide further comprises a buffering agent. In some embodiments, the buffering agent may be added in an amount of at least 10 mM. In some embodiments, the buffering agent may be added in an amount in the range of between about 10 mM to about 200 mM. In some embodiments, the buffering agent comprises citric acid monohydrate and/or trisodium citrate dehydrate.

In some embodiments, the sterile powder comprising a lyophilized polypeptide further comprises a surfactant. In some embodiments, the surfactant comprises a polysorbate. In some embodiments, the surfactant comprises polysorbate 80.

In some embodiments, the sterile powder comprising a lyophilized polypeptide further comprises a lyoprotectant. In some embodiments, the lyoprotectant comprises a sugar, such as disaccharides (e.g, sucrose). In some embodiments, the lyoprotectant comprises sucrose, trehalose, mannitol, polyvinylpyrrolidone (PVP), dextrose, and/or glycine. In some embodiments, the lyoprotectant comprises sucrose. In some embodiments, the sterile powder comprises the lyoprotectant and lyophilized polypeptide in a weight ratio of at least 1:1 lyophilized polypeptide to lyoprotectant. In some embodiments, the sterile powder comprises the lyoprotectant and lyophilized polypeptide in a weight ratio of from 1:1 to 1:10 lyophilized polypeptide to lyoprotectant. In some embodiments, the sterile powder comprises the lyoprotectant and lyophilized polypeptide in a weight ratio of 1:1, 1:2, 1:3, 1:4, 1:5, 1:6, 1:7, 1:8, 1:9, or 1:10 lyophilized polypeptide to lyoprotectant. In some embodiments, the sterile powder comprises the lyoprotectant and lyophilized polypeptide in a weight ratio of 1:6 lyophilized polypeptide to lyoprotectant. In certain embodiments of the foregoing, the sterile powder comprises lyoprotectant in an amount sufficient to stabilize the lyophilized polypeptide.

In certain embodiments of the kits disclosed herein, the injection device comprises a syringe [FIG. 19 (2)]. In some embodiments, the syringe is a 2 mL syringe. In some embodiments, the syringe is a 3 mL syringe. In certain such embodiments, the syringe is pre-filled with a reconstitution solution. In some embodiments, the reconstitution solution comprises a pharmaceutically acceptable carrier and/or excipient. In some embodiments, the pharmaceutically acceptable carrier comprises aqueous solutions such as water, physiologically buffered saline, or other solvents or vehicles such as glycols, glycerol, oils or injectable organic esters. In some embodiments, the pharmaceutically acceptable excipient comprises a pharmaceutically acceptable excipient selected from calcium phosphates, calcium carbonates, calcium sulfates, halites, metallic oxides, sugars, sugar alcohols, starch, glycols, povidones, mineral hydrocarbons, acrylic polymers, fatty alcohols, mineral stearates, glycerin, and/or lipids. In certain embodiments, the reconstitution solution comprises pharmaceutically acceptable sterile isotonic aqueous or nonaqueous solutions, dispersions, suspensions or emulsions. In certain such embodiments, the reconstitution solution comprises antioxidants, buffers, bacteriostats, and/or solutes which render the formulation isotonic with the blood of the intended recipient. In other embodiments, the reconstitution solution comprises suspending or thickening agents.

In certain embodiments of the kits disclosed herein, the kit further comprises a vial adapter [FIG. 19 (3)]. In some embodiments, the vial pre-filled with sterile powder comprising a lyophilized polypeptide attaches to one end of the vial adapter. In some embodiments, the syringe pre-filled with a reconstitution solution as disclosed herein attaches to an end of the vial adapter. In some embodiments, the syringe pre-filled with a reconstitution solution as disclosed herein and the vial pre-filled with sterile powder comprising a lyophilized polypeptide are attached to opposite ends of the vial adapter. In some embodiments, the reconstitution solution is transferred from the pre-filled syringe to the vial. In some embodiments, transfer of the reconstitution solution to the vial pre-filled with sterile powder comprising a lyophilized polypeptide reconstitutes the lyophilized polypeptide into a sterile injectable solution. In some embodiments, the lyophilized polypeptide is reconstituted into a sterile injectable solution. In some embodiments, the lyophilized polypeptide is reconstituted into a sterile injectable solution prior to use.

In other embodiments of the kits disclosed herein, the kit further comprises a pump apparatus. In certain embodiments, the pump apparatus comprises an electromechanical pumping assembly. In certain embodiments, the pump apparatus comprises a reservoir for holding a sterile injectable solution. In certain embodiments, the reservoir holds 1 mL of sterile injectable solution. In certain embodiments, the pump apparatus comprises one or more vials or cartridges comprising a sterile injectable solution. In certain embodiments, the vials or cartridges are prefilled with sterile injectable solution. In certain embodiments, the vials or cartridges comprise sterile injectable solution reconstituted from a lyophilized polypeptide. In certain embodiments, the reservoir is coupled to the vial or cartridge. In certain embodiments, the vial or cartridge holds 1-20 mL of sterile injectable solution. In certain embodiments, the electromechanical pumping assembly comprises a pump chamber. In certain embodiments, the electromechanical pumping assembly is coupled to the reservoir. In certain embodiments, the sterile injectable solution is received from the reservoir into the pump chamber. In some embodiments, the electromechanical pumping assembly comprises a plunger that is disposed such that sterile injectable solution in the pump chamber is in direct contact with the plunger. In certain embodiments, a sterile injectable solution is received from the reservoir into the pump chamber during a first pumping phase, and is delivered from the pump chamber to a subject during a second pumping phase. In certain embodiments, the electromechanical pumping assembly comprises control circuitry. In certain embodiments, control circuitry drives the plunger to (a) draw the sterile injectable solution into the pump chamber during the first pumping phase and (b) deliver the sterile injectable solution from the pump chamber in a plurality of discrete motions of the plunger during the second pumping phase, thereby delivering the therapeutic substance to the subject in a plurality of controlled and discrete dosages throughout the second pumping phase. In certain embodiments, a cycle of alternating the first and second pumping phases may be repeated until a desired dose is administered. In certain embodiments, the pump apparatus is coupled to a wearable patch. In certain embodiments, the pump apparatus is a wearable pump apparatus. In some embodiments, the pump apparatus administers a dose every 3 weeks. In some embodiments, the pump apparatus administers the dose via subcutaneous injection.

The present disclosure provides a kit used for reconstituting a lyophilized polypeptide into a sterile injectable solution. In certain embodiments, the resulting sterile injectable solution is useful in the methods disclosed herein.

In certain embodiments of the kits disclosed herein, the kit further comprises an injectable device for use in administering the sterile injectable solution parenterally [FIG. 19 (1, 2, 3, 4, and 5)]. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose. In some embodiments, the therapeutically effective dose comprises a weight based dose. In some embodiments, the weight based dose is 0.3 mg/kg. In some embodiments, the weight based dose is 0.7 mg/kg.

In some embodiments of the kits disclosed herein, the kit further comprises one or more vials or cartridges containing the lyophilized polypeptide. In some embodiments, the kit comprises at least two vials or cartridges containing the lyophilized polypeptide. In some embodiments, the kit comprises at least three vials or cartridges containing the lyophilized polypeptide. In some embodiments, the two vials can contain the same or different amounts of the lyophilized polypeptide. In some embodiments, the vials or cartridges comprise a vial or cartridge containing between 25 mg to 60 mg of lyophilized polypeptide. In some embodiments, at least one of the vials or cartridges comprise a vial or cartridge containing 60 mg of lyophilized polypeptide. In some embodiments, at least one of the vials or cartridges comprise a vial or cartridge containing 45 mg of lyophilized polypeptide. In some embodiments, at least one of the vials or cartridges comprise a vial or cartridge containing 30 mg of lyophilized polypeptide. In some embodiments, at least one of the vials or cartridges comprise a vial or cartridge containing 25 mg of lyophilized polypeptide. In some embodiments, a first vial or cartridge contains 45 mg of lyophilized polypeptide and a second vial or cartridge contains 60 mg of lyophilized polypeptide. In some embodiments, a first vial or cartridge contains 30 mg of lyophilized polypeptide and a second vial or cartridge contains 60 mg of lyophilized polypeptide. In some embodiments, a first vial or cartridge contains 30 mg of lyophilized polypeptide, a second vial or cartridge contains 45 mg of lyophilized polypeptide, and a third vial or cartridge contains 60 mg of lyophilized polypeptide. In some embodiments, a first vial or cartridge contains 25 mg of lyophilized polypeptide, a second vial or cartridge contains 45 mg of lyophilized polypeptide, and a third vial or cartridge contains 60 mg of lyophilized polypeptide. In some embodiments, the one or more vials or cartridges are refrigerated at 2-8° C.

### 7. Exemplification

The disclosure above will be more readily understood by reference to the following examples, which are included merely for purposes of illustration of certain embodiments of the present invention, and are not intended to limiting.

### Example 1: ActRIIA-Fc Fusion Proteins

A soluble ActRIIA fusion protein was constructed that has the extracellular domain of human ActRIIa fused to a human or mouse Fc domain with a minimal linker in between. The constructs are referred to as ActRIIA-hFc and ActRIIA-mFc, respectively.

ActRIIA-hFc is shown below as purified from CHO cell lines (SEQ ID NO: 23):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K

An additional ActRIIA-hFc lacking the C-terminal lysine is shown below as purified from CHO cell lines (SEQ ID NO: 41):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG

The ActRIIA-hFc and ActRIIA-mFc proteins were expressed in CHO cell lines. Three different leader sequences were considered:

(SEQ ID NO: 26)
(i) Honey bee mellitin (HBML): MKFLVNVALVFMVVYISYIYA (SEQ ID NO:
24)
(ii) Tissue plasminogen activator (TPA): MDAMKRGLCCVLLLCGAVFVSP (SEQ ID
NO: 25)
(iii) Native: MGAAAKLAFAVFLISCSSGA

The selected form employs the TPA leader and has the following unprocessed amino acid sequence:

(SEQ ID NO: 27)
MDAMKRGLCCVLLLCGAVFVSPGAAILGRSETQECLEFNANWEKDRTNQ
TGVEPCYGDKDKRRHCFATWKNISGSIEIVKQGCWLDDINCYDRTDCVE
KKDSPEVYFCCCEGNMCNEKFSYFPEMEVTQPTSNPVTPKPPTGGGTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPVPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK

This polypeptide is encoded by the following nucleic acid sequence:

(SEQ ID NO: 28)
ATGGATGCAATGAAGAGAGGGCTCTGCTGTGTGCTGCTGCTGTGTGGAG
CAGTCTTCGTTTCGCCCGGCGCCGCTATACTTGGTAGATCAGAAACTCA
GGAGTGTCTTTTTTTAATGCTAATTGGGAAAAAGACAGAACCAATCAAA
CTGGTGTTGAACCGTGTTATGGTGACAAAGATAAACGGCGGCATTGTTT
TGCTACCTGGAAGAATATTTCTGGTTCCATTGAATAGTGAAACAAGGTT
GTTGGCTGGATGATATCAACTGCTATGACAGGACTGATTGTGTAGAAAA
AAAAGACAGCCCTGAAGTATATTTCTGTTGCTGTGAGGGCAATATGTGT
AATGAAAAGTTTTCTTATTTTCCGGAGATGGAAGTCACACAGCCCACTT
CAAATCCAGTTACACCTAAGCCACCCACCGGTGGTGGAACTCACACATG
CCCACCGTGCCCAGCACCTGAACTCCTGGGGGGACCGTCAGTCTTCCTC
TTCCCCCCAAAACCCAAGGACACCCTCATGATCTCCCGGACCCCTGAGG
TCACATGCGTGGTGGTGGACGTGAGCCACGAAGACCCTGAGGTCAAGTT
CAACTGGTACGTGGACGGCGTGGAGGTGCATAATGCCAAGACAAAGCCG
CGGGAGGAGCAGTACAACAGCACGTACCGTGTGGTCAGCGTCCTCACCG
TCCTGCACCAGGACTGGCTGAATGGCAAGGAGTACAAGTGCAAGGTCTC
CAACAAAGCCCTCCCAGTCCCCATCGAGAAAACCATCTCCAAAGCCAAA
GGGCAGCCCCGAGAACCACAGGTGTACACCCTGCCCCCATCCCGGGAGG
AGATGACCAAGAACCAGGTCAGCCTGACCTGCCTGGTCAAAGGCTTCTA
TCCCAGCGACATCGCCGTGGAGTGGGAGAGCAATGGGCAGCCGGAGAAC
AACTACAAGACCACGCCTCCCGTGCTGGACTCCGACGGCTCCTTCTTCC
TCTATAGCAAGCTCACCGTGGACAAGAGCAGGTGGCAGCAGGGGAACGT
CTTCTCATGCTCCGTGATGCATGAGGCTCTGCACAACCACTACACGCAG
AAGAGCCTCTCCCTGTCTCCGGGTAAATGAGAATTC

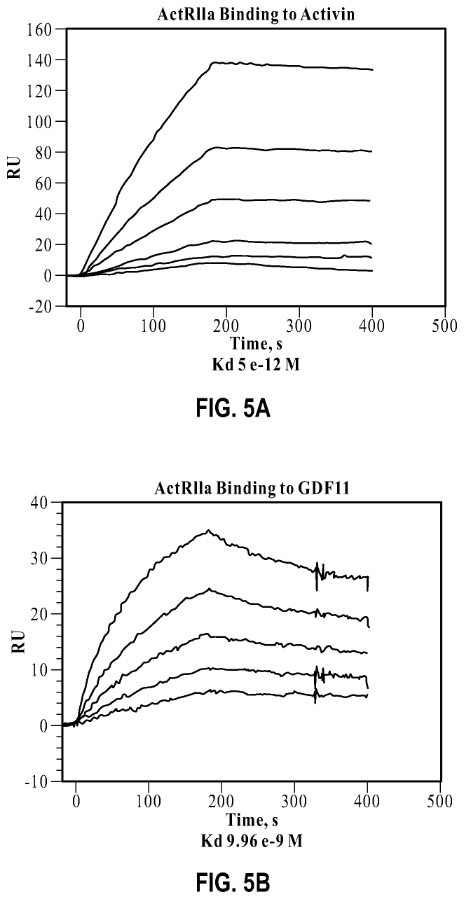

Both ActRIIA-hFc and ActRIIA-mFc were remarkably amenable to recombinant expression. As shown in FIG. 5, the protein was purified as a single, well-defined peak of protein. N-terminal sequencing revealed a single sequence of -ILGRSETQE (SEQ ID NO: 29). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIA-hFc protein was purified to a purity of >98% as determined by size exclusion chromatography and >95% as determined by SDS PAGE.

ActRIIA-hFc and ActRIIA-mFc showed a high affinity for ligands. GDF11 or activin A were immobilized on a Biacore™ CM5 chip using standard amine-coupling procedure. ActRIIA-hFc and ActRIIA-mFc proteins were loaded onto the system, and binding was measured. ActRIIA-hFc bound to activin with a dissociation constant (K_{D}) of 5×10⁻¹² and bound to GDF11 with a K_{D} of 9.96×10⁻⁹. See FIG. 5A and FIG. 5B. Using a similar binding assay, ActRIIA-hFc was determined to have high to moderate affinity for other TGF-beta superfamily ligands including, for example, activin B, GDF8, BMP6, and BMP10. ActRIIA-mFc behaved similarly.

The ActRIIA-hFc was very stable in pharmacokinetic studies. Rats were dosed with 1 mg/kg, 3 mg/kg, or 10 mg/kg of ActRIIA-hFc protein, and plasma levels of the protein were measured at 24, 48, 72, 144 and 168 hours. In a separate study, rats were dosed at 1 mg/kg, 10 mg/kg, or 30 mg/kg. In rats, ActRIIA-hFc had an 11-14 day serum half-life, and circulating levels of the drug were quite high after two weeks (11 pg/ml, 110 g/ml, or 304 g/ml for initial administrations of 1 mg/kg, 10 mg/kg, or 30 mg/kg, respectively.) In cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 2: Characterization of an ActRIIA-hFc Protein

ActRIIA-hFc fusion protein was expressed in stably transfected CHO-DUKX B11 cells from a pAID4 vector (SV40 ori/enhancer, CMV promoter), using a tissue plasminogen leader sequence of SEQ ID NO: 25. The protein, purified as described above in Example 1, had a sequence of SEQ ID NO: 23. The Fe portion is a human IgG1 Fc sequence, as shown in SEQ ID NO: 23. Protein analysis reveals that the ActRIIA-hFc fusion protein is formed as a homodimer with disulfide bonding.

The CHO-cell-expressed material has a higher affinity for activin B ligand than that reported for an ActRIIA-hFc fusion protein expressed in human 293 cells [see, del Re et al. (2004) J Biol Chem. 279(51):53126-53135]. Additionally, the use of the TPA leader sequence provided greater production than other leader sequences and, unlike ActRIIA-Fc expressed with a native leader, provided a highly pure N-terminal sequence. Use of the native leader sequence resulted in two major species of ActRIIA-Fe, each having a different N-terminal sequence.

### Example 3: Alternative ActRIIA-Fc Proteins

A variety of ActRIIA variants that may be used according to the methods described herein are described in the International Patent Application published as WO2006/012627 (see e.g., pp. 55-58), incorporated herein by reference in its entirety. An alternative construct may have a deletion of the C-terminal tail (the final 15 amino acids of the extracellular domain of ActRIIA). The sequence for such a construct is presented below (Fe portion underlined) (SEQ ID NO: 30):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMTGGGTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPVPIEKTISKAKGQPREPQVYTLPPSREEMTKNQV
SLTCLVKGFYPSDIAVEWESNGQPENNYKTTPPVLDSDGSFFLYSKLTV
DKSRWQQGNVFSCSVMHEALHNHYTQKSLSLSPGK

### Example 4: Effects of an ActRIIA Polypeptide on Pulmonary Hypertension in a Monocrotaline Rat Model

The effects of an ActRIIA-mFc fusion protein (ActRIIA-mFc homodimer as described in Example 1) and sildenafil (a phosphodiesterase-5 inhibitor approved for the treatment of PAH) were examined in a rat model of pulmonary arterial hypertension (PAH). In this model, Sprague Dawley rats received a subcutaneous injection of monocrotaline (MCT) to induce PAH 24 hours prior to start of therapy.

Rats were separated into different treatment groups (10 mice per group): 1) treatment with MCT (60 mg/kg administered i.p. as a single dose at day 1 of study) and Tris buffered saline (i.p. as 1 ml/kg, every three days) (vehicle treatment group), 2) treatment with an ActRIIA-mFe polypeptide (10 mg/kg administered i.p. every three days) and MCT (60 mg/kg administered i.p. as a single dose at day 1 of study), 3) treatment with sildenafil (30 mg/kg administered orally twice daily) and MCT (60 mg/kg administered i.p. as a single dose at day 1 of study), and 4) control rats (Tris buffered saline administered i.p. as 1 ml/kg, every three days). Rats were treated for 28 days. Body weights were recorded prior to first dose on day 1 and then weekly throughout the study.

On day 28, rats were anesthetized by an intraperitoneal injection of ketamine/xylazine (80/10 mg/kg). An incision was made in the neck, and a jugular vein was isolated and ligated anteriorly. A fluid-filled pressure catheter was introduced into the right jugular vein to measure pulmonary artery pressure (PAP). Another incision was made in the inguinal region, and femoral artery was isolated and ligated anteriorly. A Millar pressure catheter was introduced into a femoral artery to measure systolic arterial pressure, diastolic pressure, and heart rate. Mean arterial pressure and right PAP were monitored using the Notocord HEM (Croissy sur Seine, France) v3.5 data capture system for approximately 5-10 minutes until stable measurements were obtained. During the measurements, rats were maintained at approximately 37° C. on a heating pad and body temperature was monitored throughout the procedure with a rectal temperature probe. At the conclusion of the procedure, rats were euthanized, and the hearts and lungs were removed. The entire heart was weighed. Next, the atria were removed and the left ventricle with septum (LV+S) was separated from the right ventricle (RV). The ventricles were weighed separately. Hypertrophy was assessed, in part, by calculating RV/LV+S. The lungs were also weighed.

Compared to control animals, monocrotaline treated rats (vehicle treatment group) were observed to have decreased body weight, elevated PAP, right heart hypertrophy, and increased lung weight, indicating establishment of PAH. Sildenafil treated rats did not have any improvement in body weight compared to monocrotaline treated rats. However, sildenafil treatment did reduce elevated PAP by 30%, decrease right heart hypertrophy by 18.5%, and decrease lung weight by 10% compared to monocrotaline treated rats. Surprisingly, ActRIIA-mFc was found have significantly greater effects in treating PAH in this model compared to sildenafil. Specifically, while ActRIIA-mFc treatment did not show improvement in body weight, it had significant effects in treating other complications of PAH. For example, ActRIIA-Fc treatment resulted in a reduction of elevated PAP by 68%, decreased right heart hypertrophy by 47.1%, and decreased lung weight by 18.4%.

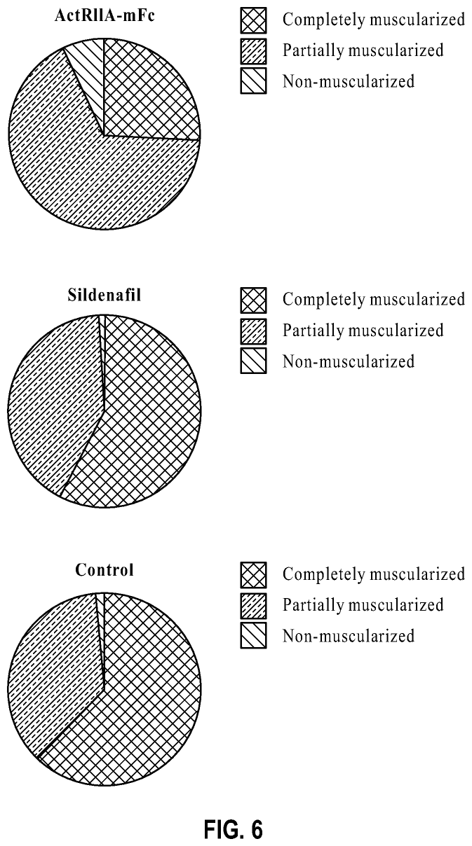

Similar trends were observed on vessel muscularity based on histopathologic scoring. After staining tissue samples to detect uSMA/elastin, 100 pulmonary arterioles, between 10 μm and 50 μm in size, per animal were categorized as non-muscularized, partially muscularized, or completely muscularized. Pulmonary arterioles from vehicle treated rats were determined to be 62.3% completely muscularized, 36.4% partially muscularized, and 1.4% non-muscularized (FIG. 6). Sildenafil treatment had only a modest effect on decreasing vessel muscularity (e.g., pulmonary arterioles being 57.9% completely muscularized, 41.6% partially muscularized, and 0.9% non-muscularized) (FIG. 6). In contrast, ActRIIA-mFc treatment resulted in significant decreases in vessel muscularity compared to sildenafil treated animals (e.g., pulmonary arterioles being 25.8% completely muscularized, 66.9% partially muscularized, and 7.3% non-muscularized compared to vehicle treated animals) (FIG. 6). Histopathological scoring of smooth muscle hypertrophy of pulmonary arterioles were also recorded as follows: 0 (normal), 1 (minimal), 2 (mild), 3 (moderate), or 4 (marked). Vehicle treated rats had an average smooth muscle hypertrophy of moderate to marked (3.8 score). Again, sildenafil treatment was observed to have a modest effect on hypertrophy with an average score of 3 (moderate). While ActRIIA-mFc treated animals were observed to have significant reduction in smooth muscle hypertrophy (average score of 1.6) compared to both vehicle and sildenafil treated animals. Overall, ActRIIA-mFc treatment significantly reduced vessel muscularity and hypertrophy in this PAH model.

Together, these data demonstrate that ActRIIA-mFc is effective in ameliorating various complications of PAH in this monocrotaline-induced model. In particular, ActRIIA-mFc had a greater effect in reducing artery pressure, right heart hypertrophy, and vascular muscularization than was observed for sildenafil, which is an approved drug for the treatment of PAH.

### Example 5: Effects of an ActRII Polypeptide on Pulmonary Hypertension in the Sugen Hypoxia Rat Model

The effects of an ActRIIA-mFc fusion protein (ActRIIA-mFc homodimer as described in Example 1 and sildenafil (a phosphodiesterase-5 inhibitor approved for the treatment of PAH) were further examined the Sugen Hypoxia model of PAH. In this model, rats receive daily doses of semaxanib and are placed in a low oxygen environment (approximately 13% oxygen) to induce PAH 24 hours prior to start of therapy.

Rats were separated into different treatment groups (10 mice per group): 1) treatment with semaxanib (200 mg/kg administered s.c. as a single dose daily)/hypoxia and Tris buffered saline (administered i.p. as 1 ml/kg, every three days) (vehicle treatment group), 2) treatment with an ActRIIA-mFc polypeptide (10 mg/kg administered i.p. every three days) and semaxanib (200 mg/kg administered s.c. as a single dose daily)/hypoxia, 3) treatment with sildenafil (30 mg/kg administered orally twice daily) and semaxanib (200 mg/kg administered s.c. as a single dose daily)/hypoxia, and 4) control rats (Tris buffered saline administered i.p. as 1 ml/kg, every three days). Rats were treated for 28 days. Body weights were recorded prior to first dose on Day 1 and then weekly throughout the study.

On day 28, rats were anesthetized by an intraperitoneal injection of ketamine/xylazine (80/10 mg/kg). An incision was made in the neck, and a jugular vein was isolated and ligated anteriorly. A fluid-filled pressure catheter was introduced into the right jugular vein to measure pulmonary artery pressure (PAP). Another incision was made in the inguinal region, and femoral artery was isolated and ligated anteriorly. A Millar pressure catheter was introduced into a femoral artery to measure systolic arterial pressure, diastolic pressure, and heart rate. Mean arterial pressure and right PAP were monitored using the Notocord HEM (Croissy sur Seine, France) v3.5 data capture system for approximately 5-10 minutes until stable measurements were obtained. During the measurements, rats were maintained at approximately 37° C. on a heating pad and body temperature was monitored throughout the procedure with a rectal temperature probe. At the conclusion of the procedure, rats were euthanized, and the hearts and lungs were removed. The entire heart was weighed. Next, the atria were removed and the left ventricle with septum (LV+S) was separated from the right ventricle (RV). The ventricles were weighed separately. Hypertrophy was assessed, in part, by calculating RV/LV+S. The lungs were also weighed.

Compared to control animals, semaxanib/hypoxia treated rats (vehicle treatment group) were observed to have decreased body weight, elevated PAP, right heart hypertrophy, and increased lung weight, indicating establishment of PAH. Sildenafil treatment reduced mean pulmonary artery pressure by 22.4% and decreased right heart hypertrophy by 10% compared to vehicle treated animals. Again, ActRIIA-mFc treatment was found have significantly greater effects in treating PAH in this model compared to sildenafil. For example, ActRIIA-mFc treatment resulted in a reduction of mean pulmonary artery pressure by 51.3% and decreased right heart hypertrophy by 53.5% compared to vehicle treated animals.

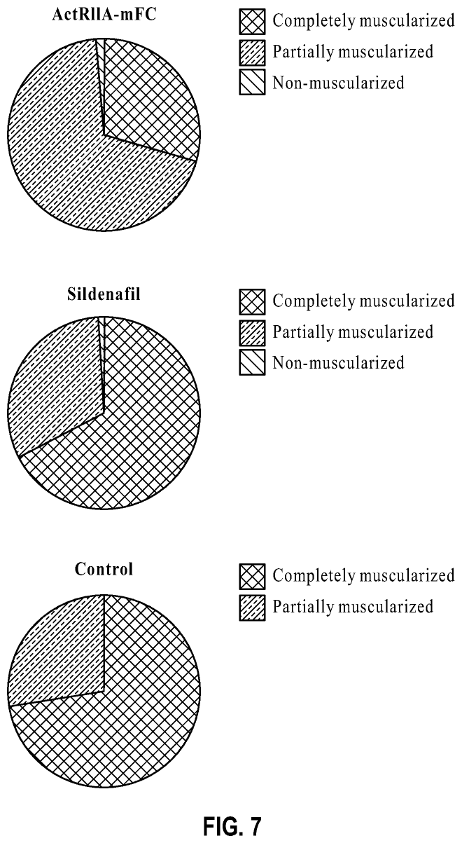

Similar trends were observed on vessel muscularity based on histopathologic scoring. After staining tissue samples to detect αSMA/elastin, 100 pulmonary arterioles, between 10 μm and 50 μm in size, per animal were categorized as non-muscularized, partially muscularized, or completely muscularized. Pulmonary arterioles from vehicle treated rats were determined to be 72.5% completely muscularized, 27.4% partially muscularized, and 0.1% non-muscularized (FIG. 7). Sildenafil treatment had only a modest effect on decreasing vessel muscularity (e.g., pulmonary arterioles being 67.4% completely muscularized, 31.6% partially muscularized, and 1.0% non-muscularized) compared to vehicle treated animals (FIG. 7). In contrast, ActRIIA-mFc treatment resulted in significant decreases in vessel muscularity compared to sildenafil treated animals (e.g., pulmonary arterioles being 29.3% completely muscularized, 69.3% partially muscularized, and 1.4% non-muscularized compared to vehicle treated animals) (FIG. 7). Histopathological scoring of smooth muscle hypertrophy of pulmonary arterioles were also recorded as follows: 0 (normal), 1 (minimal), 2 (mild), 3 (moderate), or 4 (marked). Vehicle treated rats had an average smooth muscle hypertrophy of moderate to marked (3.6 score). Again, sildenafil treatment was observed to have a modest effect on hypertrophy with an average score of 3 (moderate). While ActRIIA-mFc treated animals were observed to have significant reduction in smooth muscle hypertrophy (average score of 1.4) compared to sildenafil treated animals. Overall, ActRIIA-mFc treatment significantly reduced vessel muscularity and hypertrophy in this PAH model.

Together, these data demonstrate that ActRIIA-mFc is effective in ameliorating various complications of PAH in the Sugen Hypoxia model. In particular, ActRIIA-mFc had a greater effect in reducing artery pressure, right heart hypertrophy, and vessel muscularization than was observed for sildenafil, which is an approved drug for the treatment of PAH.

### Example 6: Methods Used in 24-Week Placebo-Controlled Trial Using an ActRII Polypeptide Treatment in Patients with Pulmonary Arterial Hypertension

The efficacy and safety of sotatercept (an ActRIIA-hFc fusion protein as described in Example 1) as a concomitant treatment in patients with pulmonary arterial hypertension on background pulmonary hypertension therapy was examined in a 24-week placebo-controlled trial. Adults receiving background therapy were randomized into one of the three following groups: (1) subcutaneous sotatercept 0.3 mg/kg; (2) subcutaneous sotatercept 0.7 mg/kg; or (3) subcutaneous placebo every 3 weeks. The primary end point was change in pulmonary vascular resistance.

### Patients

Eligible patients had confirmed pulmonary arterial hypertension (Group 1 of the updated Pulmonary Hypertension Classification) in World Health Organization (WHO) functional class II or III, excluding portopulmonary, schistosomiasis, and human immunodeficiency virus-associated subtypes. Patients were receiving stable pulmonary arterial hypertension standard of care therapy for at least 90 days before and throughout the study (standard-of-care was determined by the treating physician and not per-protocol standardized; patients were treated with mono-, double, or triple therapy with combinations of endothelin-receptor antagonists, phosphodiesterase 5 inhibitors, soluble guanylate cyclase stimulators, prostacyclin analogues, or prostacyclin receptor agonists). Sensitivity analysis was performed to account for these differences in background therapy. All patients provided informed consent.

### Trial Design and Oversight

The trial was a phase 2, double-blind, randomized, multicenter trial with a 24-week placebo-controlled treatment period, followed by an 18-month active drug extension. Here, we report the topline results of the 24-week placebo-controlled period.

Patients were randomized at 43 centers in eight countries. A steering committee designed the study in collaboration with the sponsor. An institutional review board or independent ethics committee approved the protocol at each site. An independent data monitoring committee reviewed unblinded safety data 6 weeks after the first study dose.

Initially, eligible patients were stratified according to baseline WHO functional class and randomized, using computerized Interactive Response Technology, in a 1:1:1 ratio to one of three treatment groups on top of standard of care: placebo, sotatercept 0.3 mg/kg, or sotatercept 0.7 mg/kg. During the study, this ratio was changed to 3:3:4 to increase the power for the 0.7 mg/kg sotatercept group; seven patients had been enrolled at the time of this change. Sotatercept or placebo (saline) was given by subcutaneous injection every 21 days (cumulative drug exposure detailed in Table 3). Safety and efficacy were assessed at screening and every 3 weeks for 24 weeks. Adverse events were recorded from screening until the end of primary treatment study visit, 8 weeks after the last dose of study drug. Dose modifications were planned for leukopenia, thrombocytopenia, neutropenia, and changes in blood pressure and hemoglobin levels following guidance from the European Health Authority. These adverse events had been reported in previous sotatercept trials (Table 4). Patients who discontinued or were withdrawn were asked to return for the end-of-study visit. Additionally, 2D Doppler echocardiography was performed at baseline and 24 weeks and read in a central core laboratory.

TABLE 3
Dose Modification Criteria-Delay, Reduction, and Discontinuation.

| | |
|---|---|
| Event at day of visit | Action |

Adverse Event

| | |
|---|---|
| Any related adverse event | Dose delay for 3 weeks; if not improved to Grade ≤1, |
| Grade ≥2b | discontinue treatment; if improved to Grade ≤1, |
| | restart study drug and reduce by one dose level |
| Third dose reduction required | Discontinue treatment*^{†} |
| due to adverse event^{‡} | |

Leukopenia

| | |
|---|---|
| White blood cell count | Dose delay until resolved to ≤Grade 1 (>3000/mm³) |
| <3000/mm³ | or baseline values, then reduce dose by one level |
| (<3.0 × 10⁹/L) Grade 2 | |
| White blood cell count | Dose delay for 3 weeks; if not resolved to ≤Grade 1 |
| <2000/mm³ (<2.0 × 10⁹/L) | (>3000/mm³), discontinue treatment; if resolved, |
| Grade 3 | restart dosing and reduce by one dose level |

Thrombocytopenia

| | |
|---|---|
| Platelet count <75,000/mm³ | Dose delay until resolved to >75,000/mm³ or baseline, |
| (<75.0 × 10⁹/L) | and then reduce dose by one dose level |
| Platelet count <50,000/mm³ | Dose delay for 1 cycle; if not resolved to >75,000/ |
| (<50.0 × 10⁹/L) Grade 3 | mm³, discontinue treatment; if resolved, restart |
| | dosing and reduce by one dose level |

Neutropenia

| | |
|---|---|
| Absolute neutrophil count | Dose delay until resolved to ≤Grade 1 (>1500/mm³) |
| <1500/mm³ (<1.5 × 10⁹/L) | or baseline, and then reduce dose by one dose level |
| Grade 2 | |
| Absolute neutrophil count | Discontinue treatment |
| <1000/mm³ (<1.0 × 10⁹/L) | |
| Grade 3 | |

Blood pressure

| | | |
|---|---|---|
| Systolic blood pressure (SBP) | 1) | Dose delay for 3 weeks and treat with |
| ≥140 mmHg or diastolic blood | | antihypertensive(s) until target of SBP |
| pressure (DBP) ≥90 mmHg | | <140 mmHg and DBP <90 mmHg is reached. |
| | | If target BP is achieved at next visit, restart |
| | | study drug at same dose level |
| | 2) | If target of SBP <140 mmHg and DBP <90 |
| | | mmHg is not achieved after treatment with |
| | | antihypertensive(s) and dose delay for one cycle, |
| | | dose delay for another cycle and adjust |
| | | antihypertensive regimen. If target is achieved at |
| | | next cycle, restart study drug at same dose level |
| | 3) | Discontinue study drug treatment if target is not |
| | | achieved after two dose delays |

Hemoglobin (Hgb)

| Hgb ≥18.0 g/dL at any time | Phlebotomy until Hgb <16.0 g/dL; delay dose for one | |
|---|---|---|
| | cycle; at next cycle, reduce study drug by one dose | |
| | level | |
| Hgb increase <2.0 g/dL after | Continue study drug at same dose | |
| any cycle (not influenced by | | |
| RBC transfusions) compared | | |
| with pre-dose Hgb of the | | |
| previous sotatercept | | |
| administration AND Hgb | | |
| <18.0 g/dL | | |
| Hgb increase ≥2.0 g/dL after | If Hgb is: | |
| any cycle (not influenced by | 1) | <15.0 g/dL - continue dosing as per protocol |
| RBC transfusions) compared | 2) | ≥15.0 but <16.0 g/dL - delay dose for one cycle; |
| with pre-dose Hgb of the | | restart study drug at the same dose level |
| previous study drug | 3) | ≥16.0 g/dL but <18.0 g/dL - delay dose for one |
| administration. If Hgb is | | cycle; at the next cycle, if Hgb is <16.0 g/dL, |
| <15.0 g/dL | | continue study drug at same dose level |
| | 4) | ≥16.0 g/dL, reduce study drug by one dose level |
| | | and restart study drug |

*The sponsor could terminate study treatment or a dose level after consultation with the investigator and the data monitoring committee at any time for safety or administrative reasons.
^{†}The sponsor would terminate the study if the occurrence of severe adverse events or other findings suggested unacceptable risk to the health of the participants.
^{‡}Pharmacokinetic modeling of the hemoglobin response to sotatercept 0.7 mg/kg predicted that patients were most likely to down titrate between cycles 2 and 3, which led to the definition of patients in the evaluable set requiring six administrations of the same dose.
DBP: diastolic blood pressure;
Hgb: hemoglobin;
RBC: red blood cell;
SBP: systolic blood pressure

TABLE 4
Study End Points.

| | | Results |
|---|---|---|
| | | stated in |
| | | this report |
| Type | End points | (Y/N) |
| Primary | Change in pulmonary vascular resistance | Y |
| end point | from baseline to 24 weeks | |
| Key | Change in 6-minute walk distance from | Y |
| secondary | baseline to 24 weeks, also measured at | |
| end point | weeks 3, 9, 15, and 21 | |
| Other | Change from baseline to 24 weeks in NT- | Y |
| secondary | proBNP, also measured at weeks 3, 12, and | |
| end points | 21 | |
| | Change from baseline to 24 weeks in | Y |
| | tricuspid annular plane systolic excursion | |
| | Change from baseline to 24 weeks in WHO | Y |
| | functional class, also assessed every 3 weeks | |
| | Clinical worsening from baseline to 24 | Y |
| | weeks | |
| | Change from baseline to 24 weeks in quality | Y |
| | of life CAMPHOR¹ and SF-36² | |
| | Safety and tolerability assessments based on | Y |
| | adverse events, clinical laboratory values, | |
| | vital signs, and electrocardiograms | |
| | Population pharmacokinetics | Y |
| Exploratory | Change from baseline in TGF-β ligands and | N |
| end points | other relevant serum biomarkers | |
| | Change from baseline to 24 weeks in | N |
| | echocardiographic parameters | |
| | Correlation of clinical efficacy with BMPR2 | N |
| | expression in peripheral blood mononuclear | |
| | cells | |
| | Correlation of clinical efficacy with sex | Y |
| | hormone levels in males and females | |
| | Composite end point of clinical | Y |
| | improvement - the number of patients who | |
| | exhibit at least two of the following criteria: | |
| | Any Improvement in WHO functional | |
| | class or maintenance of functional class II | |
| | Improvement in NT Pro-BNP by at least | |
| | 30% | |
| | Improvement in 6-minute walk distance | |
| | by at least 30 m | |

BMPR2: bone morphogenetic protein receptor 2;
CAMPHOR: Cambridge Pulmonary Hypertension Outcome Review;
NT-pro-BNP: N-terminal pro-brain natriuretic peptide;
SF-36: short form 36;
TGF-β: transforming growth factor beta;
WHO: World Health Organization

### End Points

The primary end point was the change in pulmonary vascular resistance from baseline to 24 weeks. The key secondary end point was the change in 6 minute walk distance from baseline to 24 weeks. Other secondary end points included: change from baseline to 24 weeks in N terminal pro brain natriuretic peptide (NT proBNP), echocardiography measures (tricuspid annular plane systolic excursion, right ventricular fractional area change), WHO functional class, and safety (full list of end points provided in Table 5).

Pulmonary vascular resistance was calculated using mean pulmonary artery pressure, pulmonary artery wedge pressure, and cardiac output, all measured by right heart catheterization at screening and at week 24 of the treatment period. Echocardiograms (2-dimentional; 2D) were acquired according to a pre-defined protocol and approved, reviewed, and analyzed by a blinded central laboratory (BioTel Research, Rockville, MD. US). End points were assessed at baseline and selected study visits. Adverse events were graded using the National Cancer Institute Common Toxicity Criteria for Adverse Events (CTCAE) system (defined in the Supplementary Appendix).

TABLE 5
Study Drug Exposure in the Placebo-Controlled Period for the Full
Analysis Set

| | | Sotatercept | Sotatercept | Sotatercept |
|---|---|---|---|---|
| | Placebo | 0.3 mg/kg | 0.7 mg/kg | all doses |
| Measure | (N = 32) | (N = 32) | (N = 42) | (N = 74) |
| Mean duration of | 165.2 | 163.4 | 160.3 | 161.6 |
| exposure* | (84, 183) | (48, 188) | (41, 184) | (41, 188) |
| Mean number of | 7.7 ± 0.7 | 7.5 ± 1.1 | 7.1 ± 1.5 | 7.3 ± 1.3 |
| treatment cycles | | | | |
| Mean of total | 0.0 | 142.2 | 343.1 | 256.2 |
| dose | | (44.0, 243.9) | (91.6, 731.1) | (44.0, 731.1) |
| administered | | | | |
| (mg) | | | | |

*Mean duration of exposure is given in days

### Statistical Analyses

A minimum required sample size of 26 per treatment group was determined based on the following: an expected baseline pulmonary vascular resistance of 800 dyn·s/cm5 (standard deviation of 400 dyn·s/cm5), a 30% reduction at 24 weeks (240 dyn·s/cm5) for sotatercept treatment groups and no change for the placebo group, with a two-sided alpha of 0.10 and 80% power. However, the results reported here represent a two-sided alpha of 0.05 with 95% power. The confidence intervals have not been adjusted for multiplicity and cannot be used to infer definitive treatment effects.

Change in pulmonary vascular resistance and 6 minute walk distance from baseline to 24 weeks were analyzed using analysis of covariance (ANCOVA), with the randomization factor and baseline value as covariates. Data normality was tested using the Shapiro-Wilk test, followed by an aligned Wilcoxon rank-sum test in the event of a significant P value, using the same covariates of baseline WHO functional class and baseline value of the analyzed end point. The median is reported alongside mean as a more representative value for any data with abnormal or borderline abnormal distribution. Other end points were summarized with descriptive statistics and tested using ANCOVA where appropriate. Sensitivity analysis was performed for the primary and key secondary end points (Table 6). Safety analysis included the full analysis set.

TABLE 6
Sensitivity Analysis for the Full Analysis Set

| | | Sotatercept 0.3 | | Sotatercept 0.7 | | Sotatercept all | |
|---|---|---|---|---|---|---|---|
| | Placebo | mg/kg (N = 32) | | mg/kg (N = 42) | | doses (N = 74) | |
| | (N = 32) | | LS mean | | LS mean | | LS mean |
| | LS Mean | LS Mean | difference | LS Mean | difference | LS Mean | difference |
| Measure | (SE) | (SE) | (95% CI) | (SE) | (95% CI) | (SE) | (95% CI) |
| Pulmonary | −30.3 | −171.1 | −140.7 | −259.4 | −229.1 | −221.1 | −190.9 |
| vascular | (36.8) | (34.5) | (−239.6, −41.9) | (30.6) | (−322.2, −136.0) | (23.2) | (−276.6, −105.1) |
| resistance - | | | | | | | |
| dyn · s/cm⁵ | | | | | | | |
| 6-Minute | 21.3 | 54.8 | 33.6 | 51.9 | 30.6 | 53.2 | 31.9 |
| Walk | (9.2) | (9.1) | (8.1, 59.0) | (8.2) | (6.4, 54.8) | (6.1) | (10.2, 53.5) |
| Distance - | | | | | | | |
| m | | | | | | | |

Worst Case Sensitivity Analysis

| | | | | | | | |
|---|---|---|---|---|---|---|---|
| Pulmonary | −15.8 ± | −162.1 ± | −146.3 | −247.8 ± | −232.0 | −210.6 ± | −194.9 |
| vascular | 32.9 | 32.8 | (−238.5, −54.1) | 28.8 | (−318.5, −145.4) | 21.9 | (−273.9, −115.8) |
| resistance - | | | | | | | |
| dyn · s/cm⁵ | | | | | | | |
| 6-Minute | 27.0 ± | 58.3 ± | 31.4 | 47.4 ± | 20.4 | 52.1 ± | 25.1 |
| Walk | 9.3 | 9.3 | (5.2, 57.6) | 8.2 | (−4.1, 45.0) | 6.1 | (3.0, 47.3) |
| Distance - | | | | | | | |
| m | | | | | | | |

### Example 7: Results from 24-Week Placebo-Controlled Trial Using an ActRII Polypeptide Treatment in Patients with Pulmonary Arterial Hypertension

### Baseline Characteristics

A total of 106 patients were randomized to receive placebo (n=32), sotatercept 0.3 mg/kg (n=32), or sotatercept 0.7 mg/kg (n=42) on top of standard of care. Baseline data were similar among groups, showing a relatively young patient population (mean age±SD; 48.3±14.3 years) with moderate to severe pulmonary arterial hypertension. Most patients were on triple therapy (55.7%), of whom almost one-third were receiving parenteral (intravenous or subcutaneous) prostacyclin analogues.

### Primary End Point

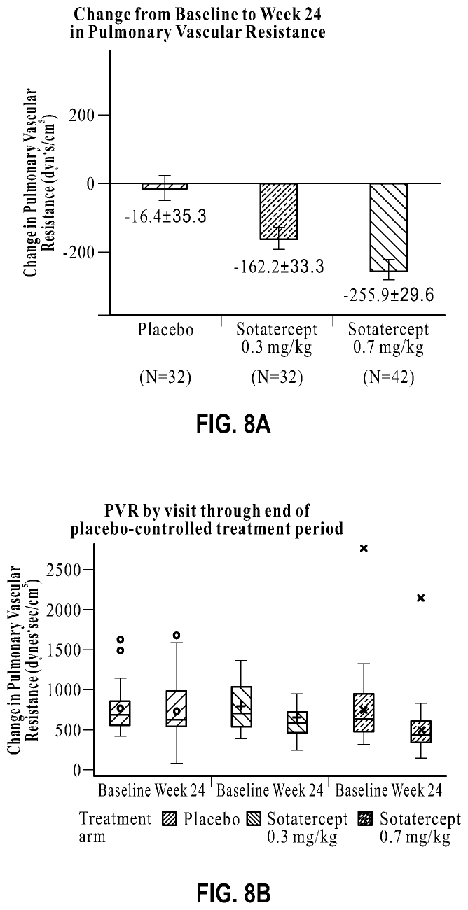

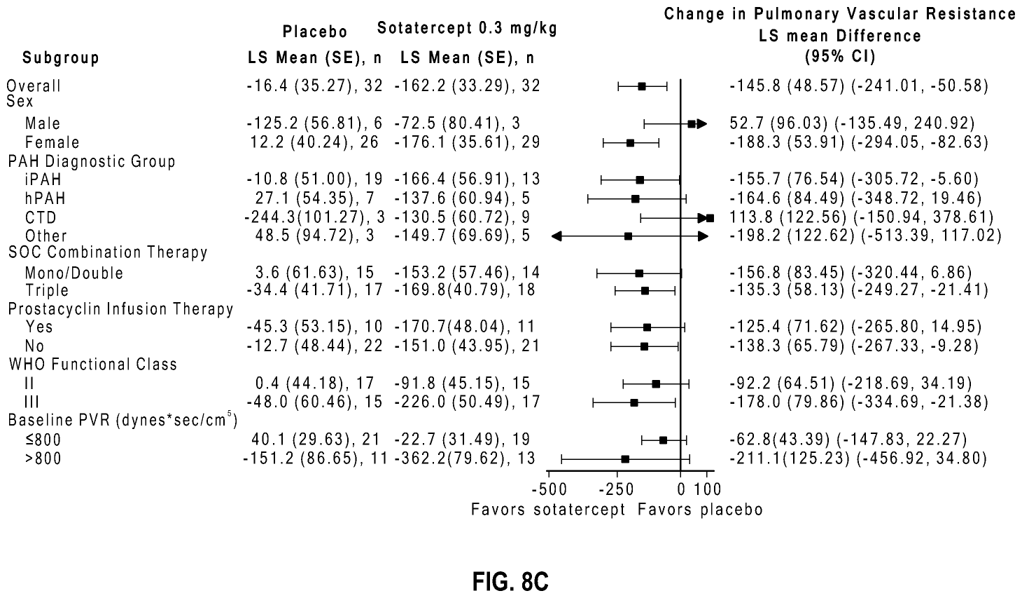

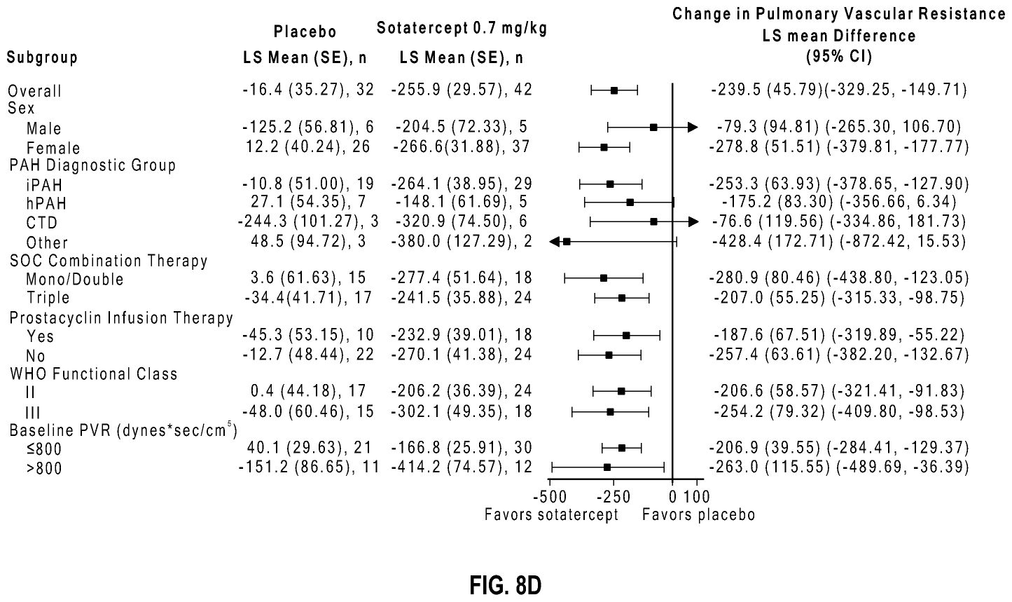

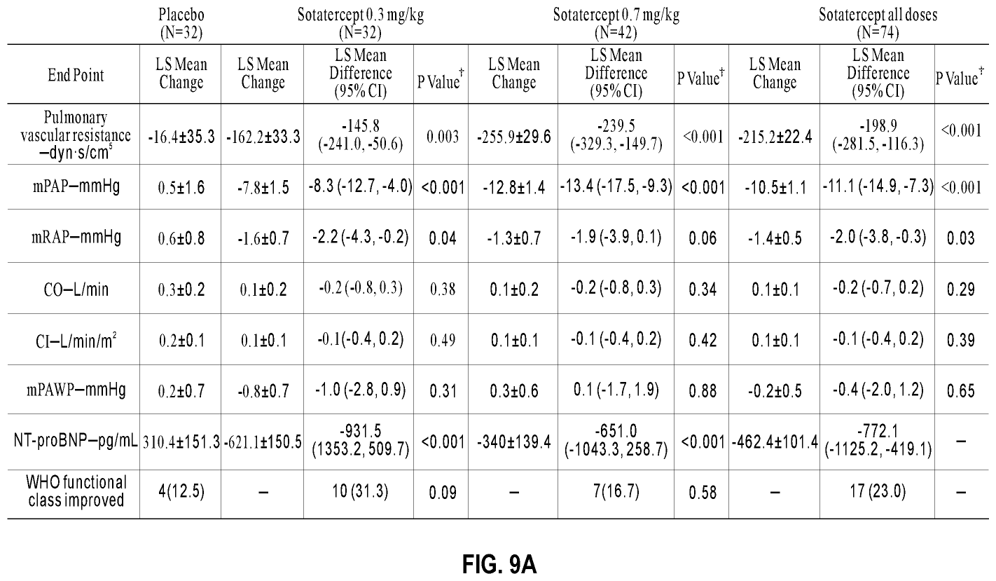

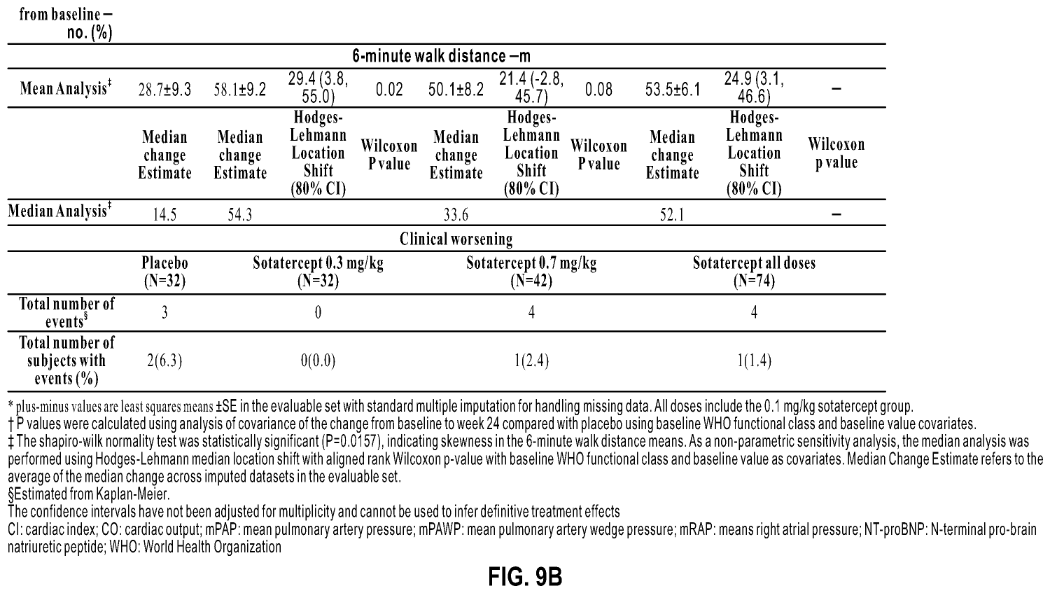

In the full analysis set at week 24, the least squares mean change from baseline in pulmonary vascular resistance was a decrease of 162.2 dyn·s/cm5 in the sotatercept 0.3 mg/kg group and 255.9 dyn·s/cm5 in the sotatercept 0.7 mg/kg group, compared with 16.4 dyn·s/cm5 for placebo (least squares mean difference, 95% confidence intervals [CI]: sotatercept 0.3 mg/kg 145.8 dyn·s/cm5, 241.0 to 50.6, P=0.003; sotatercept 0.7 mg/kg 239.5 dyn·s/cm5, −329.3 to −149.7, P<0.001; FIGS. 8A-8D, FIG. 9A, and FIG. 9B). This treatment effect remained consistent across patients receiving mono, double, or triple therapy, including parenteral prostacyclin. The reduction in pulmonary vascular resistance was driven by a decreased least squares mean difference versus placebo in mean pulmonary artery pressure of 8.3 mmHg (−12.7 to −4.0) for the sotatercept 0.3 mg/kg group and 13.4 mmHg (−17.5 to −9.3) for the sotatercept 0.7 mg/kg group (FIG. 9A and FIG. 9B). In contrast, pulmonary artery wedge pressure and cardiac output showed minimal changes across all treatment groups.

### Secondary Efficacy End Points

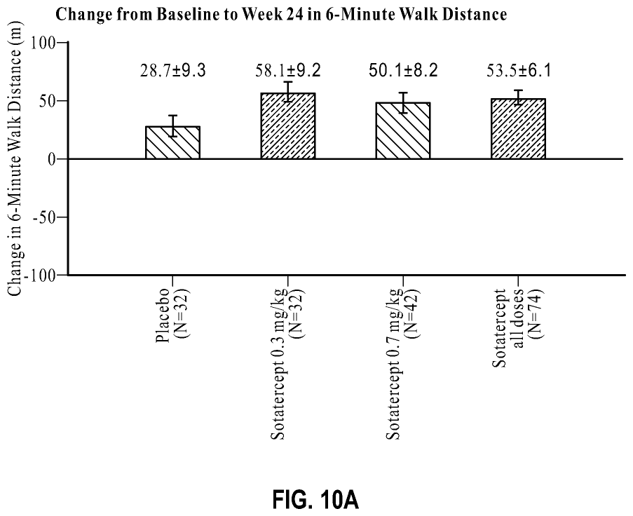

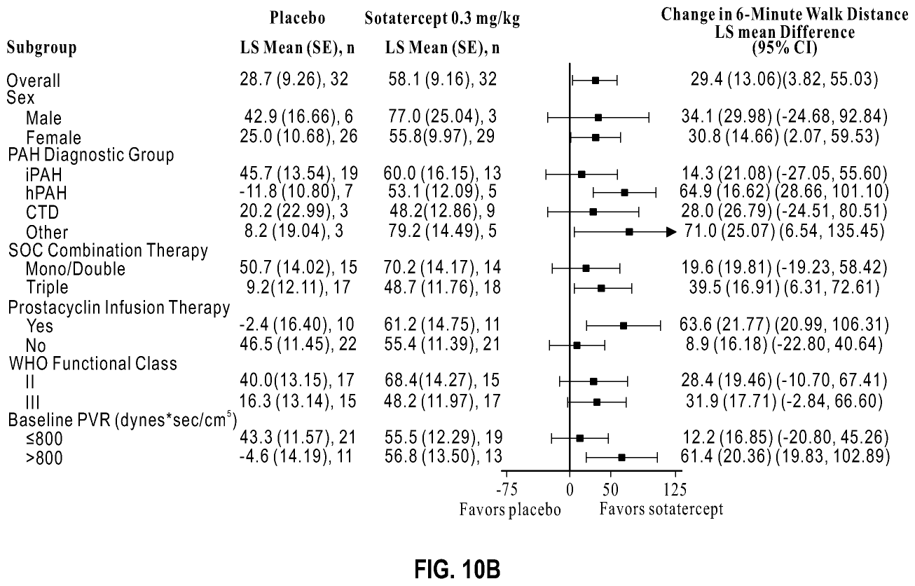

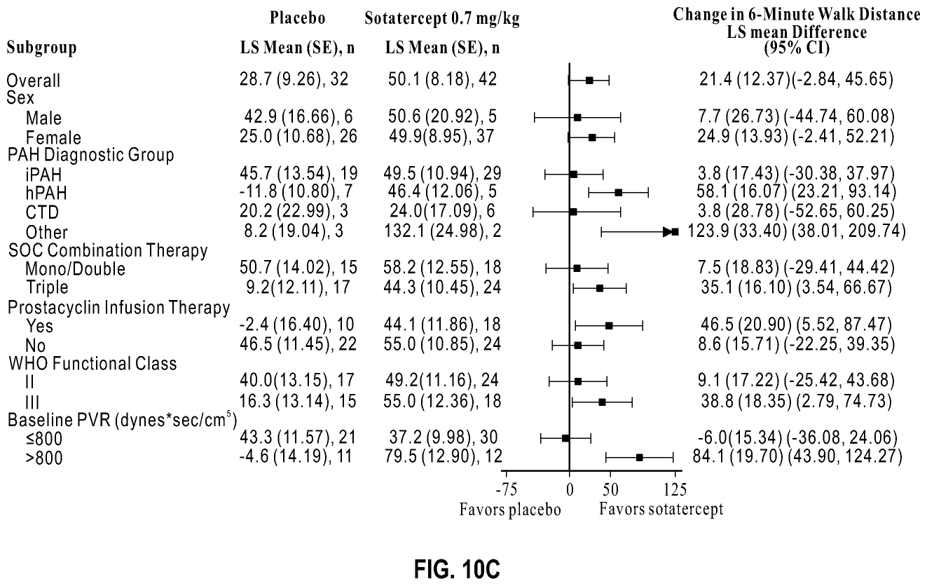

In the full analysis set, the least squares mean change from baseline in 6-minute walk distance at week 24 was a similar increase of 58.1 m in the sotatercept 0.3 mg/kg group and 50.1 m in the sotatercept 0.7 mg/kg group, compared with a 28.7 m increase in the placebo group (FIG. 9A, FIG. 9B, and FIGS. 10A-10C). In a prespecified analysis, both sotatercept dosage groups were combined and compared with placebo, producing a least squares mean difference of 24.9 m (95% CI: 3.1 to 46.6).

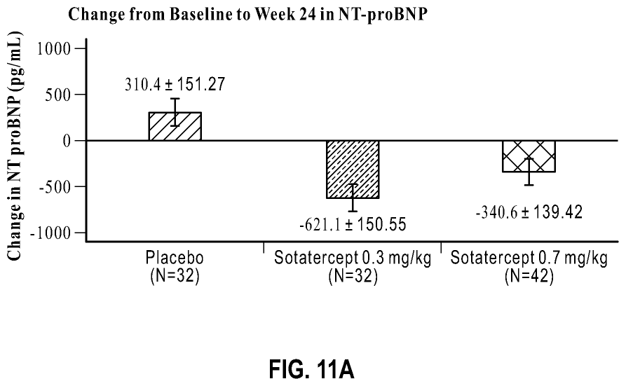

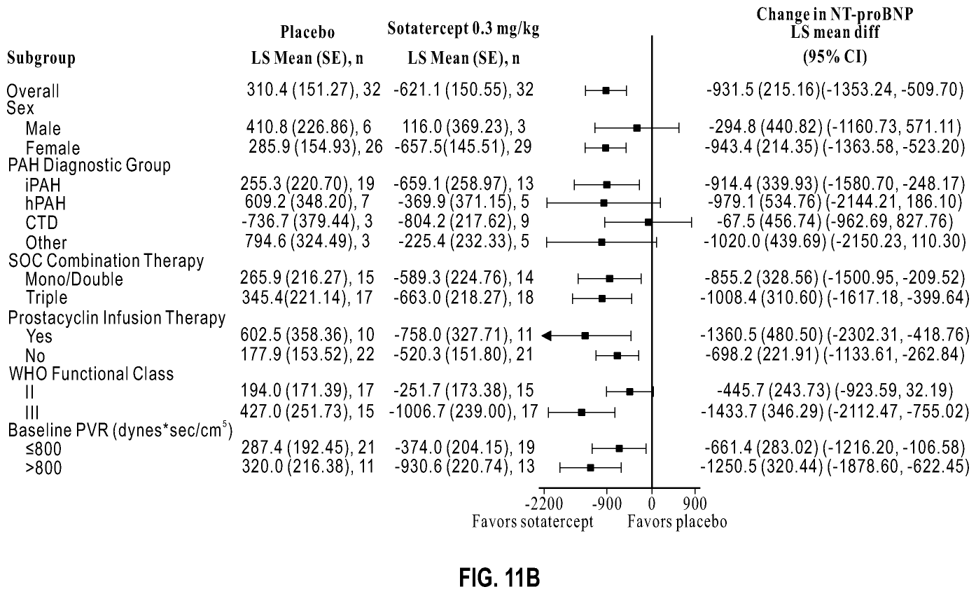

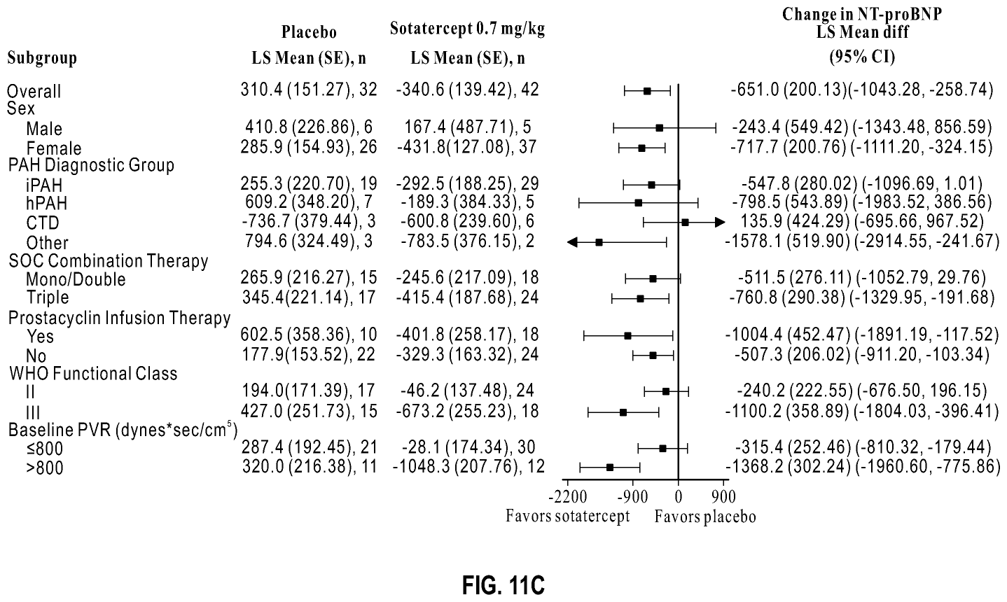

At week 24, the least squares mean levels of NT-proBNP decreased from baseline by 621.1±150.5 pg/mL in the sotatercept 0.3 mg/kg group, and 340.6±139.4 pg/mL in the sotatercept 0.7 mg/kg group, compared with an increase of 310.4±151.3 pg/mL with placebo (least squares mean difference, 95% CI: sotatercept 0.3 mg/kg −931.6, 1353.2 to 509.7; sotatercept 0.7 mg/kg 651.0, 1043.3 to 258.7; FIG. 9A, FIG. 9B, and FIGS. 11A-11C). WHO functional class improved from baseline by at least one class in four patients (13.3%) on placebo, ten patients (32.3%) on sotatercept 0.3 mg/kg, and seven patients (19.5%) on sotatercept 0.7 mg/kg (FIG. 9A and FIG. 9B).

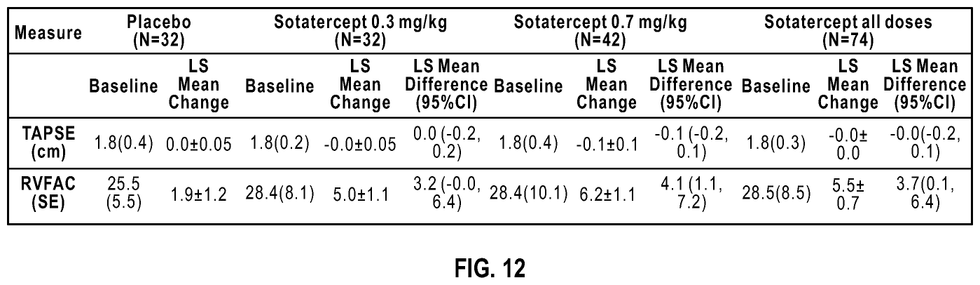

Echocardiography measures at week 24 show the least squares mean levels of right ventricular fractional area change increased from baseline in the sotatercept 0.3 mg/kg group by 5.0±1.1%, and in the sotatercept 0.7 mg/kg group by 5.9±1.0%, compared with an increase of 1.8±1.2% in the placebo group (least squares mean difference 95% CI: sotatercept 0.3 mg/kg 3.2, −0.02 to 6.4; sotatercept 0.7 mg/kg 4.1, 1.1 to 7.2). The mean change in tricuspid annular plane systolic excursion did not differ between sotatercept and placebo (FIG. 12).

### Safety

The most commonly reported adverse events throughout the study occurred to a similar degree across all treatment groups (Table 7). Serious adverse events and dose-modification criteria are detailed in Table 3 and Table 8.

Thrombocytopenia was a chosen term and events did not necessarily signify a clinical definition of concern. Thrombocytopenia was the most common adverse event of special interest, occurring in five patients (11.9%) in the sotatercept 0.7 mg/kg group. Of these, four patients had CTCAE Grade 1 platelet counts (<150×109/L) at baseline and shifted to Grade 2 (50−75×109/L) following treatment. The three remaining Grade 2 patients were managed per protocol with dose interruptions (n=2) followed by dose reduction (n=1) until their platelet counts returned to Grade 1 levels. The remaining patient had platelet counts within the normal range at baseline, which subsequently decreased to Grade 1 at the last study visit. In the sotatercept 0.3 mg/kg group, thrombocytopenia was reported for two patients (9.3%). One patient started the study with a low platelet count that declined further during treatment but increased following two dose interruptions. The second patient had normal platelet counts at baseline, decreasing to Grade 1 during treatment and rising again after dose reduction. There were no events of thrombocytopenia-related bleeding reported during the study, and no patient required platelet infusion.

Mean hemoglobin levels increased from baseline 8 days after the first study dose in both the sotatercept 0.3 mg/kg (0.9±0.7 g/dL) and sotatercept 0.7 mg/kg (1.1±0.6 g/dL) groups, while levels decreased in the placebo group (0.3±0.7 g/dL). By week 24, hemoglobin levels had not changed from baseline in the placebo group (0.0±1.1 g/dL), but had increased in the sotatercept 0.3 mg/kg (1.2±1.2 g/dL) and sotatercept 0.7 mg/kg (1.5±1.1 g/dL) groups (Table 7). Hemoglobin increase was reported as an adverse event for one patient (3.1%) in the sotatercept 0.3 mg/kg group and six patients (14.3%) in the sotatercept 0.7 mg/kg group. Grade 3 adverse events were reported for 5, 3, and 11 patients (15.6%, 9.4%, and 26.2%) in the placebo, sotatercept 0.3 mg/kg, and sotatercept 0.7 mg/kg groups, respectively.

Overall, three patients were withdrawn per protocol due to hemoglobin levels rising above 18 g/dL and undergoing protocol-mandated phlebotomy-one in the sotatercept 0.3 mg/kg group and two in the 0.7 mg/kg group. One patient discontinued due to thrombocytopenia and one withdrew consent, both in the sotatercept 0.7 mg/kg group. One patient died during the study, in the sotatercept 0.7 mg/kg group, due to a cardiac arrest. This was deemed unrelated to study treatment by the investigator, based on the individual's other ongoing health conditions.

TABLE 7
Adverse Events and Hematology Variables Through End of
Placebo-Controlled Treatment Period

| | | | Sotatercept | | Sotatercept | |
|---|---|---|---|---|---|---|
| | Placebo | | 0.3 mg/kg | | 0.7 mg/kg | |
| Variable | (N = 32) | | (N = 32) | | (N = 42) | |
| Adverse event (AE) | 28 | (87.5) | 29 | (90.6) | 34 | (81.0) |

AEs in ≥10% patients

| Headache | 5 | (15.6) | 8 | (25.0) | 6 | (14.3) |
|---|---|---|---|---|---|---|
| Diarrhea | 4 | (12.5) | 7 | (21.9) | 6 | (14.3) |
| Peripheral edema | 5 | (15.6) | 3 | (9.4) | 5 | (11.9) |
| Dizziness | 3 | (9.4) | 5 | (15.6) | 4 | (9.5) |
| Fatigue | 6 | (18.8) | 2 | (6.3) | 4 | (9.5) |
| Hypokalemia | 4 | (12.5) | 3 | (9.4) | 5 | (11.9) |
| Nausea | 4 | (12.5) | 3 | (9.4) | 5 | (11.9) |
| AE of special interest | 0 | (0.0) | 3 | (9.4) | 6 | (14.3) |
| Leukopenia | 0 | (0.0) | 1 | (3.1) | 1 | (2.4) |
| Neutropenia | 0 | (0.0) | 0 | (0.0) | 1 | (2.4) |
| Thrombocytopenia | 0 | (0.0) | 2 | (6.3) | 5 | (11.9) |
| Serious AE | 3 | (9.4) | 2 | (6.3) | 10 | (23.8) |
| Serious related AE | 1 | (3.1) | 0 | (0.0) | 2 | (4.8) |
| AE leading to treatment | 1 | (3.1) | 2 | (6.3) | 3 | (7.1) |
| discontinuation | | | | | | |
| AE leading to study | 2 | (6.3) | 1 | (3.1) | 3 | (7.1) |
| discontinuation | | | | | | |
| AE leading to death | 0 | (0.0) | 0 | (0.0) | 1 | (2.4)‡ |
| Hemoglobin increase | 0 | (0.0) | 1 | (3.1) | 6 | (14.3) |
| Change in hematology | | | | | | |
| variables from baseline | | | | | | |
| to week 24 | N = 30 | | N = 31 | | N = 36 | |
| Hemoglobin - g/dL | 0.0 | ± 1.1 | 1.2 | ± 1.2 | 1.5 | ± 1.1 |
| Platelet count - ×10⁹/L | −6.3 | ± 29.1† | 12.1 | ± 47.7 | −12.1 | ± 49.8 |

*Adverse events data are number of patients with events (%) in the safety set. Plus-minus values are means ± SD.
†29 patients were included in the placebo group platelet count analysis.
‡This patient died due to a cardiac arrest deemed unrelated to study treatment. Pre-existing risk factors included hypertension, type 2 diabetes, chronic obstructive pulmonary disease, hyperlipidemia, atrial fibrillation, congestive heart disease, and ischemic heart disease, and concomitant medications included furosemide, spironolactone, ambrisentan, tadalafil, apixaban, bisoprolol, digoxin, glicazide, linagliptin, pravastatin, and tiotropium

TABLE 8
Serious Treatment-Emergent Adverse Events by System Organ Class

| | | | Sotatercept | | Sotatercept | |
|---|---|---|---|---|---|---|
| Event by system organ class and | Placebo | | 0.3 mg/kg | | 0.7 mg/kg | |
| preferred term - no. (%) | N = 32 | | N = 32 | | N = 42 | |
| Total number of subjects with | 3 | (9.4) | 2 | (6.3) | 10 | (23.8) |
| events | | | | | | |
| Blood and lymphatic system | 0 | (0.0) | 0 | (0.0) | 1 | (2.4) |
| disorders | | | | | | |
| Leukopenia | 0 | (0.0) | 0 | (0.0) | 1 | (2.4) |
| Neutropenia | 0 | (0.0) | 0 | (0.0) | 1 | (2.4) |
| Cardiac disorders | 2 | (6.3) | 1 | (3.1) | 3 | (7.1) |
| Cardiac arrest | 1 | (3.1) | 0 | (0.0) | 1 | (2.4) |
| Pericardial effusion | 0 | (0.0) | 0 | (0.0) | 1 | (2.4) |
| Right ventricular failure | 1 | (3.1) | 1 | (3.1) | 0 | (0.0) |
| Tachycardia | 0 | (0.0) | 0 | (0.0) | 1 | (2.4) |
| Eye disorders | 0 | (0.0) | 0 | (0.0) | 1 | (2.4) |
| Chorioretinopathy | 0 | (0.0) | 0 | (0.0) | 1 | (2.4) |
| General disorders and | 0 | (0.0) | 0 | (0.0) | 2 | (4.8) |
| administration site conditions | | | | | | |
| Peripheral edema | 0 | (0.0) | 0 | (0.0) | 1 | (2.4) |
| Pyrexia | 0 | (0.0) | 0 | (0.0) | 1 | (2.4) |
| Infections and infestations | 1 | (3.1) | 0 | (0.0) | 3 | (7.1) |
| Bronchitis | 0 | (0.0) | 0 | (0.0) | 1 | (2.4) |
| Gastroenteritis | 1 | (3.1) | 0 | (0.0) | 0 | (0.0) |
| Influenza | 0 | (0.0) | 0 | (0.0) | 1 | (2.4) |
| Respiratory tract infection | 0 | (0.0) | 0 | (0.0) | 1 | (2.4) |
| Injury, poisoning, and procedural | 0 | (0.0) | 0 | (0.0) | 1 | (2.4) |
| complications | | | | | | |
| Femur fracture | 0 | (0.0) | 0 | (0.0) | 1 | (2.4) |
| Investigations | 0 | (0.0) | 0 | (0.0) | 1 | (2.4) |
| Red blood cell count increased | 0 | (0.0) | 0 | (0.0) | 1 | (2.4) |
| Nervous system disorders | 1 | (3.1) | 0 | (0.0) | 1 | (2.4) |
| Migraine | 1 | (3.1) | 0 | (0.0) | 0 | (0.0) |
| Syncope | 0 | (0.0) | 0 | (0.0) | 1 | (2.4) |
| Product issues | 0 | (0.0) | 0 | (0.0) | 1 | (2.4) |
| Device breakage | 0 | (0.0) | 0 | (0.0) | 1 | (2.4) |
| Respiratory, thoracic, and | 0 | (0.0) | 1 | (3.1) | 0 | (0.0) |
| mediastinal disorders | | | | | | |
| Epistaxis | 0 | (0.0) | 1 | (3.1) | 0 | (0.0) |
| Vascular disorders | 0 | (0.0) | 0 | (0.0) | 1 | (2.4) |
| Hypotension | 0 | (0.0) | 0 | (0.0) | 1 | (2.4) |

### Example 8: Other Echocardiography Results from 24-Week Placebo-Controlled Trial Using an ActRII Polypeptide Treatment in Patients with Pulmonary Arterial Hypertension

The effects of sotatercept (an ActRIIA-hFc fusion protein as described in Example 1) on right ventricular-pulmonary artery (RV-PA) coupling and right ventricular function was evaluated in patients in the 24-week placebo-controlled trial. The same methods and patients were used as described in Examples 6 and 7. The right ventricle (RV) and pulmonary arteries (PA) become uncoupled as RV function deteriorates in PAH, a consequence of pulmonary vascular remodeling which is ultimately fatal. Preclinically, sotatercept, has been shown to reverse right heart remodeling and improve right heart structure and function and acts by suppressing ActRIIA signaling and therefore rebalancing BMPR2 signaling.

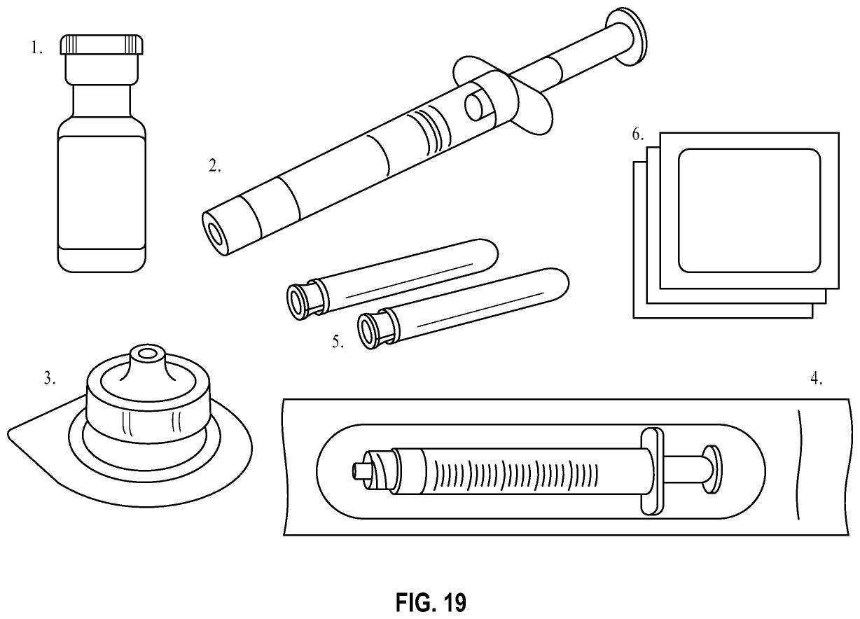

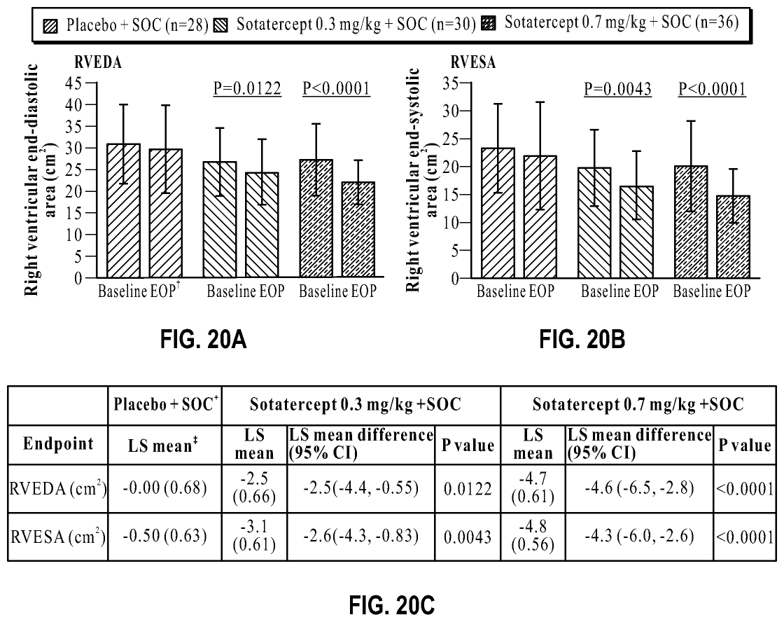

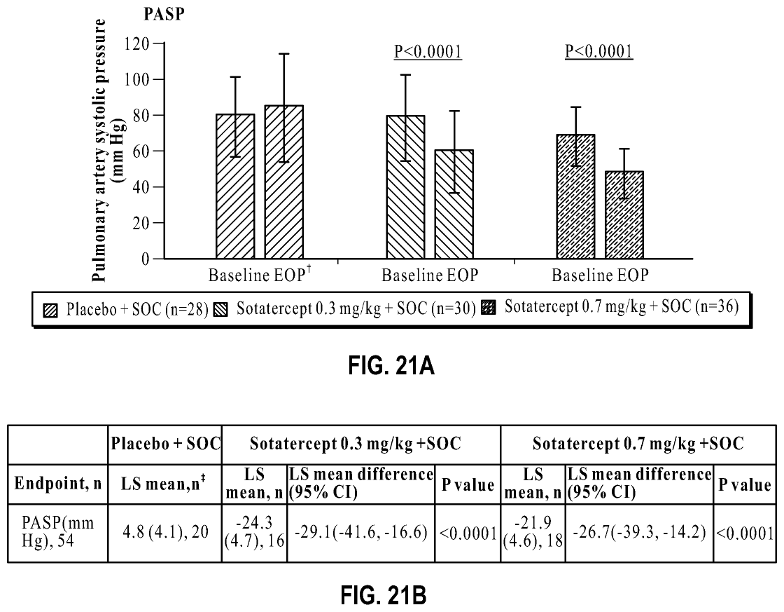

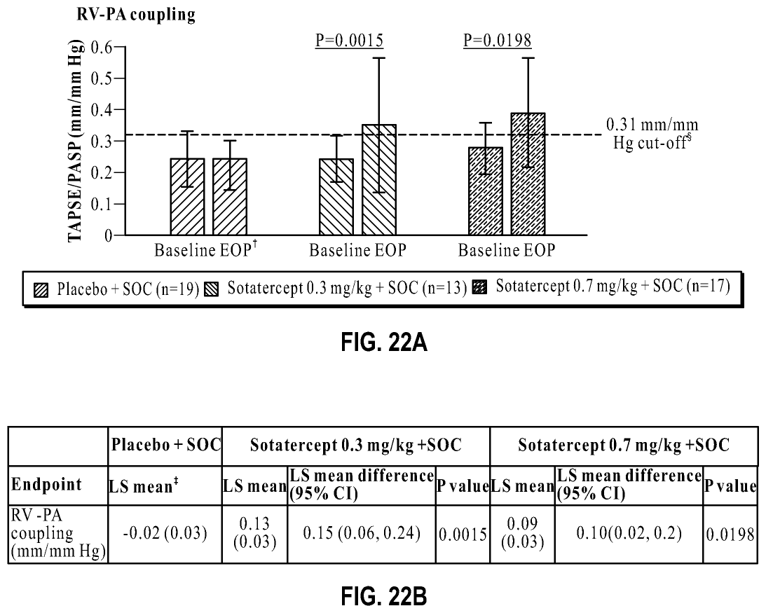

Restoration of RV-PA coupling and RV function is a crucial aim of PAH treatment. RV-PA coupling can be estimated non-invasively as a ratio of TAPSE/PASP values. A TAPSE/PASP ratio of ≥0.31 mm/mm Hg is associated with a better prognosis and reduced risk of clinical worsening. Accordingly, RV-PA coupling was assessed by TAPSE/PASP, using the TAPSE measurements shown in Example 7. See FIG. 12. Significant improvement was seen in LS mean (SE) change from baseline to week 24 in RV-PA coupling, RVEDA, RVESA, PASP and RAP in both sotatercept dose level groups vs placebo. See FIG. 20A, FIG. 20B, FIG. 20C, FIG. 21A, and FIG. 21B. No changes were seen in CO. For RV-PA coupling, all patients started below 0.31 mm/mmHg prognostic threshold; both treatment arms improved above that threshold by 24 weeks while the placebo arm remained below. See FIG. 22A and FIG. 22B.

In the 24-week placebo-controlled trial, treatment with sotatercept compared to placebo was associated with statistically significant improvements in RV-PA coupling and RV function.

### Example 9: Methods Used in Examples 10-14

### Fusion Protein and Neutralizing Antibodies

RAP-011 is an ActRIIA-mFc fusion protein and was constructed as described in Example 1. Anti-activin A antibody and an antibody with dual specificity for myostatin and GDF11 (RK35) were modified internally for use in mice by substitution of murine IgG2a Fc. Anti-activin B antibody was generated internally.

### Animal Models

Adult male Sprague-Dawley (SD) and Wistar (WI) rats (150-180 gm) were purchased from Envigo, Indianapolis, IN, for use as SU/Hx/Nx and MCT rat models, respectively. All experimental procedures were approved by the Institutional Animal Care and Use Committee. The SU/Nx/Nx model was established by a single subcutaneous injection of vascular endothelial growth factor receptor antagonist Sugen5416 (20 mg/kg; Cayman) with simultaneous exposure to normobaric hypoxia (10% O₂) for 3 weeks, followed by normoxia (21% O₂) for 7 weeks. The MCT model was established by a single subcutaneous injection of MCT (60 mg/kg, Torris) followed by 4 week exposure to normoxia (10% 02). Right ventricular hypertrophy was induced by pressure overload in a pulmonary artery banding (PAB) model of pulmonary hypertension. Briefly, 10-week-old male C57BL/6 mice were anesthetized with ketamine (100 mg/kg) and xylazine (10 mg/kg). Endotracheal intubation was performed, and the endotracheal tube was connected to a small animal ventilator at 100 breaths/min and a tidal volume of 0.2 ml. Mice were placed in the supine position, a midline incision was made, and the chest cavity was entered at the second intercostal space to expose the pulmonary artery. A 25-gauge blunt needle was tied against the pulmonary artery, the needle was promptly removed, and the wound was closed in two layers.

### Hemodynamic Measurements

Rats were anesthetized with 3-4% isoflurane and placed on controlled heating pads. Right ventricular systolic pressure (RVSP) was measured by advancing a 2F curve tip pressure transducer catheter (SPR-513, Millar Instruments) into the right ventricle via the right jugular vein under 1.5-2% isofluorane anesthesia. Cardiac output was assessed by advancing a 2 Fr microtipped PV catheter (SPR 838, Millar Instruments) into the left ventricle through the right carotid artery under 1.5-2% isofluorane anesthesia. Cardiac index was calculated by dividing cardiac output by body weight. Total pulmonary vascular resistance index (TPRI) was estimated by dividing RVSP by cardiac index. En-bloc heart and lungs were collected and lungs were perfused with physiological saline via the right ventricular outflow tract to flush the blood cells from the pulmonary circulation. Right ventricular hypertrophy was determined by calculating the weight ratio of right ventricular free wall to the combined left ventricle and septum (Fulton's Index).

### Echocardiography

Echocardiography was performed with a Vevo 3100 imaging system with MX201 scanhead (VisualSonics, Toronto, ON, Canada) on rats anesthetized with 3-4% isoflurane and maintained with 1.5-2% isoflurane. B-Mode, M-Mode and pulse-wave Doppler flow imaging were performed in each rat at the end of weeks 5 and 9. Briefly, rats were placed supine on a heated platform and allowed to breathe spontaneously. The right ventricular outflow tract was visualized using a modified parasternal long axis view. Pulmonary artery acceleration time (PAAT) was measured as the time from start to peak velocity of blood flow in the lumen of the main pulmonary artery distal to the pulmonary valve as obtained from the pulse-wave doppler recording. B-Mode parasternal short-axis view of a mid-ventricular cross section of the heart was visualized at the level of the papillary muscles. Right ventricular wall thickness (RVWT) was measured using M-mode in a modified parasternal long-axis view through the aortic valve. RV fractional area change (RV-FAC) was measured using a B-mode apical four-chamber view. Tricuspid annular plane systolic excursion (TAPSE) was obtained from the apical four-chamber view directing the M-mode doppler beam through the lateral annulus of the tricuspid valve plane. For each parameter, measurements from three individual heartbeats per animal were taken and averaged.

### Cell Culture

Human pulmonary artery endothelial cells (PAECs) and pulmonary artery smooth muscle cells (PASMCs) were obtained from Lonza. PAECs were maintained in vascular cell basal medium (PCS100-030^{T}M, ATCC®) supplemented with endothelial cell growth kit (PCS-100-041™, ATCC®), phenol red (PCS-999-001™, ATCC®) and penicillin-streptomycin-amphotericin B solution (PCS-999-002™, ATCC®). PASMCs were maintained in vascular cell basal medium supplemented with vascular smooth muscle cell growth kit (PCS-100-0421™, ATCC®), phenol red, and penicillin-streptomycin-amphotericin B solution. Prior to treatment, both cell types were synchronized overnight in low serum medium (vascular cell basal medium supplemented with 0.1% fetal bovine serum, phenol red, and penicillin-streptomycin-amphotericin B solution). PAEC conditioned medium (PAEC-CM) was generated by exposing PAECs at approximately 80-90% confluence in low serum medium (0.1% fetal bovine serum) to 3% hypoxia for 24 hours in a ProOx C21 hypoxia unit (Biosheperix). To determine whether activins or GDFs mediate effects of conditioned medium from hypoxia-exposed PAECs on PASMC proliferation, approximately 6000 PASMCs were plated per well in a 96-well plate. PASMCs synchronized overnight were treated with either low serum medium or PAEC-CM with or without sotatercept (ACE-011) or ligand-neutralizing antibodies. ACE-011 and antibodies were preincubated with PAEC conditioned medium for 30 min at room temperature prior to PASMC treatment. After 48 hours treatment, cell proliferation was assessed with a bromodeoxyuridine-based assay kit (6813, Cell Signaling Technology) according to the manufacturer's instructions.

### Example 10: Effects of Preventive RAP-011 Treatment in Models of Acquired and Genetic PH

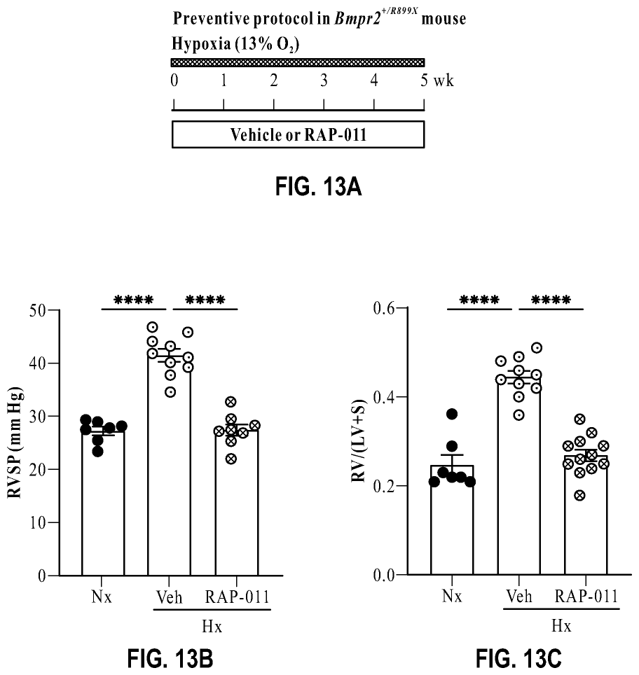

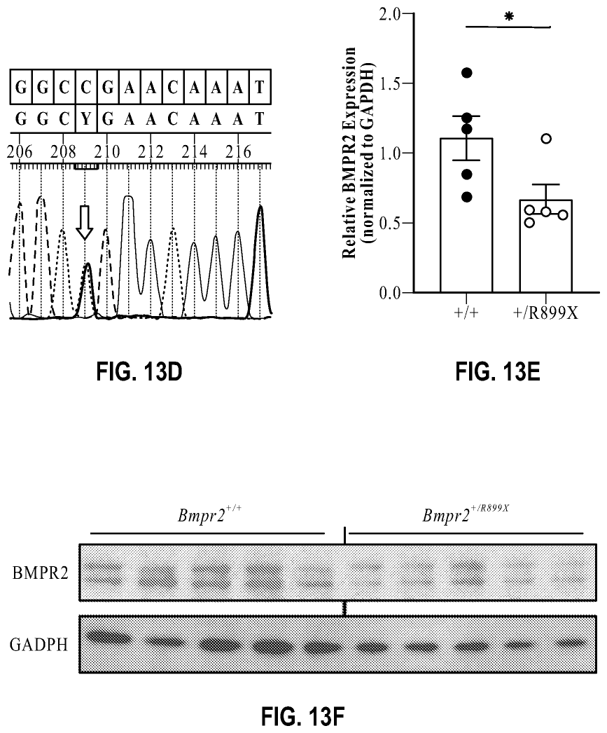

We tested RAP-011 in a mouse model of PH caused by Bmpr2 haploinsufficiency since the BMPR2 pathway is heavily implicated in pulmonary vascular homeostasis and familial PAH. In Bmpr2^{+/R899X} mice, exposure to hypoxia for 5 weeks starting at 4 months of age (FIG. 13A) significantly elevated right ventricular systolic pressure and induced right ventricular hypertrophy whereas preventive treatment with RAP-011 completely normalized these parameters (FIGS. 13B and 13C). Analysis of genomic DNA confirmed that these mice possess a heterozygous nucleotide substitution at the expected position, and immunoblotting confirmed reduced levels of BMPR2 protein in lung (FIG. 13D-F). Together, these results indicate that preventive treatment with RAP-011 produces strong beneficial effects not only in widely-used models of acquired PH but also in a model of heritable PH caused by Bmpr2 haploinsufficiency.

### Example 11: Therapeutic RAP-011 Treatment Reverses Vascular Remodeling in Severe Experimental PAH

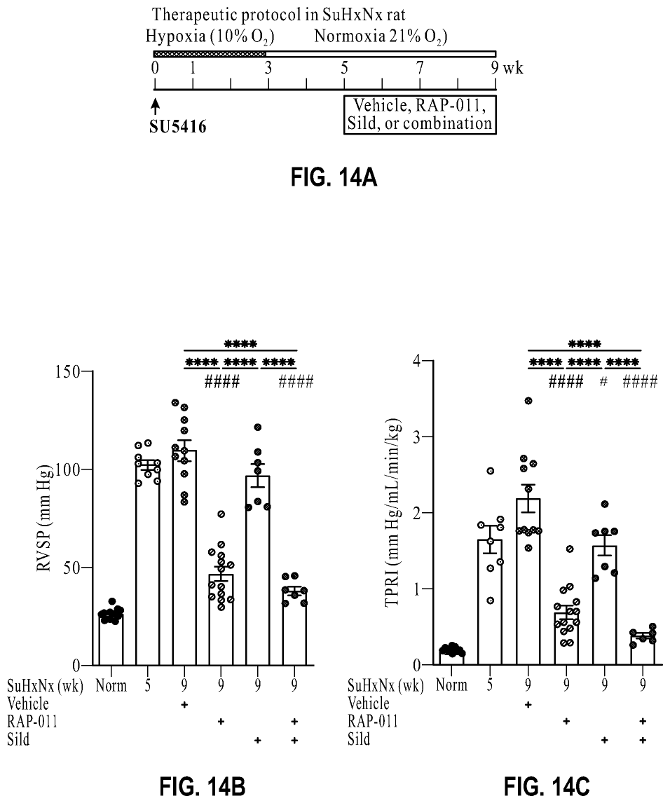

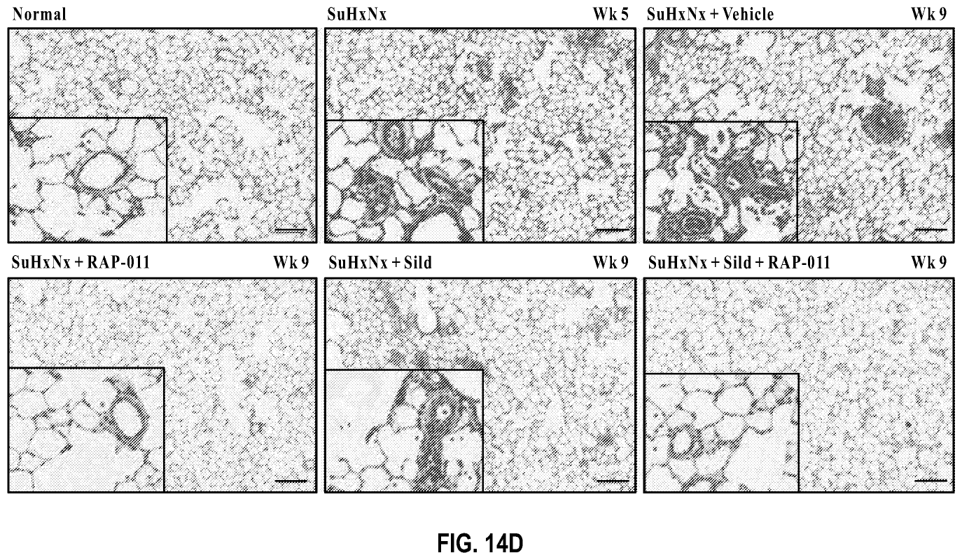

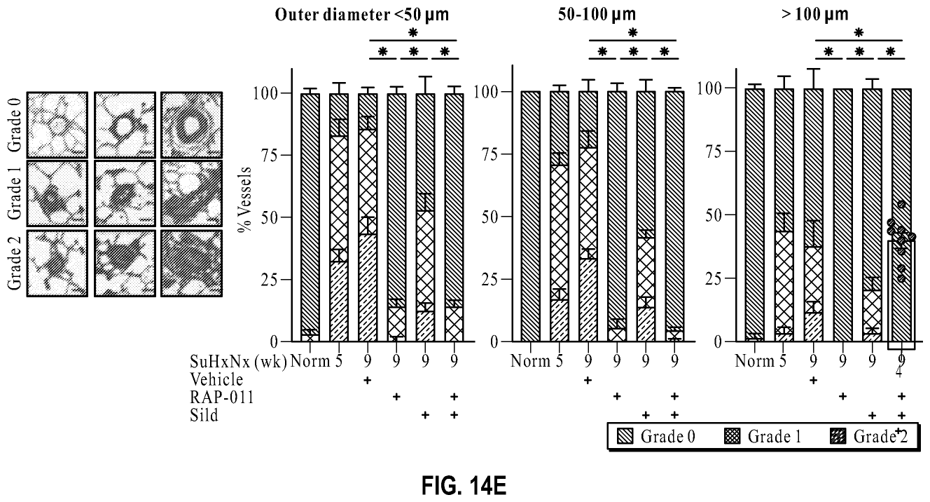

We next investigated whether RAP-011 is effective when administered after development of pathology in a Sugen-hypoxia-normoxia (SuHxNx) rat model of severe angio-obliterative PAH (FIG. 14A). This model mimics important features of human PAH, including pulmonary vascular remodeling, perivascular pulmonary inflammation, marked right ventricular dysfunction, and a progressive course culminating in severe occlusive arteriopathy. It has been observed that this model—with a normoxic phase of progression included—is largely unresponsive to current PAH treatments and therefore broadly consistent with therapeutic efficacy in patients. Here, we confirmed that hemodynamic parameters including right ventricular systolic pressure and total pulmonary resistance index were significantly impaired in untreated SuHxNx rats at the onset of therapeutic treatment (week 5) and remained undiminished at week 9 (FIG. 14B and FIG. 14C). Therapeutic treatment with RAP-011 starting at week 5 markedly improved hemodynamic deficits by week 9 compared to untreated SuHxNx rats at the same time point (FIG. 14B and FIG. 14C). RAP-011 exerted effects superior to those of the vasodilator sildenafil, used here as a representative of standard therapy. Most importantly, RAP-011 in combination with sildenafil produced significantly greater improvement in hemodynamic parameters than sildenafil alone (FIG. 14B and FIG. 14C).

Histologic examination of lung tissue in SuHxNx rats revealed changes consistent with the foregoing hemodynamic results. Pulmonary vascular remodeling was prominent in untreated SuHxNx rats by week 5 with plexiform lesions leading to varying degrees of vascular occlusion (FIG. 14D and FIG. 14E). At week 9, there was significantly greater prevalence of the most severe grade of vascular occlusion compared to the earlier time point, consistent with disease progression during this interval (FIG. 14D and FIG. 14E). Therapeutic treatment with RAP-011 starting at week 5 almost completely reversed vascular remodeling by week 9 compared to untreated SuHxNx rats at the same time point (FIG. 14D and FIG. 14E). As determined by blinded histologic assessment, RAP-011 reversed vascular occlusion more effectively than sildenafil, and RAP-011 in combination with sildenafil produced significantly greater improvement in vascular occlusion than sildenafil alone. Together, these findings indicate that RAP-011 monotherapy is superior to sildenafil monotherapy—and RAP-011 more effective in combination therapy than standard of care—for reversal of hemodynamic impairment and pulmonary vascular remodeling in this rat model of severe angio-obliterative PAH.

### Example 12: RAP-011 Exerts Antiproliferative and Other Effects by Sequestering Multiple Ligands in the Activin-GDF Family

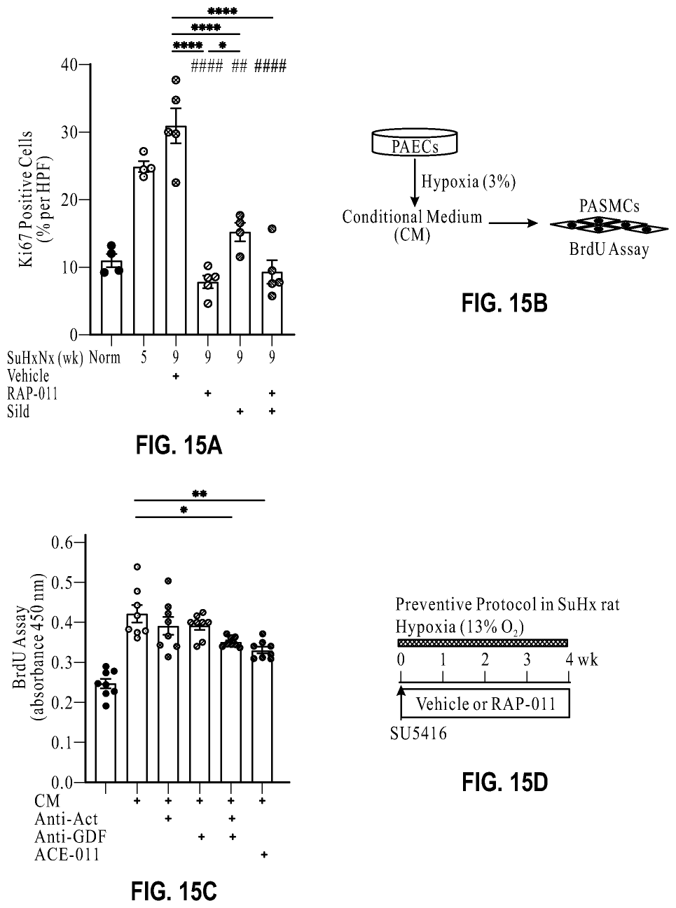

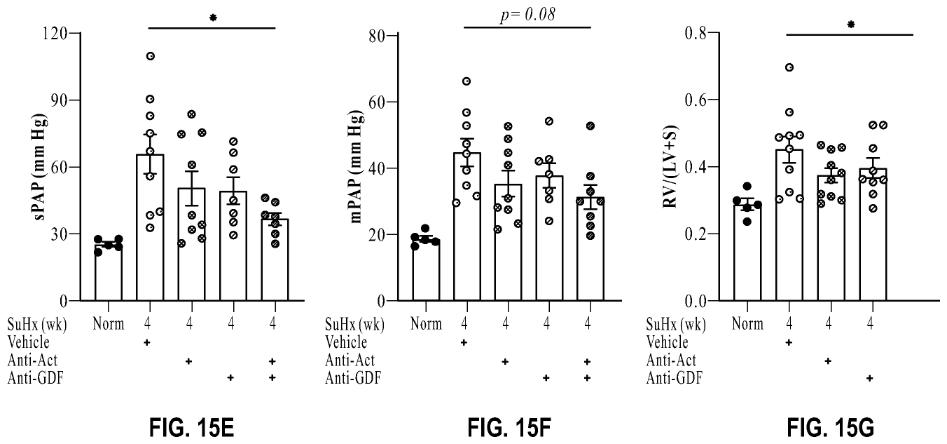

We next examined effects of RAP-011 on vascular cell proliferation in the lung. In the SuHxNx rat model of severe PAH, proliferation of pulmonary vascular cells as determined by immunostaining for Ki67 was substantially increased under vehicle-treated conditions compared to normal and normalized by therapeutic treatment with RAP-011, either as monotherapy or in combination with sildenafil, but only partially normalized with sildenafil alone (FIG. 15A). These results indicate that therapeutic treatment with RAP-011 is superior to sildenafil—and more effective when combined with standard therapy than standard therapy alone—for reversal of pulmonary vascular cell proliferation in this SuHxNx model of severe PAH.

We then investigated the contribution of certain TGF-β superfamily ligands to the antiproliferative effects of sotatercept (or RAP-011), which binds activins and GDFs with high affinity and slow off-rates advantageous for ligand sequestration. We used antibodies against these ligands to investigate potential ligand contributions to antiproliferative effects of sotatercept on bromodeoxyuridine labeling in a human cell-based model of vascular injury. In this assay, primary pulmonary arterial smooth muscle cells were exposed to conditioned medium from primary pulmonary arterial endothelial cells grown under hypoxic conditions in vitro (FIG. 15B). Treatment of conditioned medium with sotatercept produced an antiproliferative effect that was equaled in magnitude by treatment of conditioned medium with a combination of antibodies against activins (separate antibodies against activin A and activin B) and GDFs (antibody with dual specificity against GDF8 and GDF11) but not by separate antibody treatments (FIG. 15B). These data indicate that sequestration of multiple Smad2/3-pathway ligands plays a role in the antiproliferative effects of sotatercept (or RAP-011) in this in vitro model of vascular injury.

We used a similar approach to probe which ligands are responsible for beneficial effects of RAP-011 on cardiopulmonary parameters in a preventive SuHxNx rat model (FIG. 15C). In this in vivo model, elevated hemodynamic parameters such as systolic pulmonary artery pressure and mean pulmonary artery pressure were normalized more effectively by combined treatment with antibodies against activins, GDF8, and GDF11 than separate antibody treatments (FIGS. 15D and 15E). Combined treatment with these antibodies also normalized right ventricular hypertrophy (Fulton index) more effectively than separate antibody treatments (FIG. 15F). Together, these results indicate that sequestration of multiple Smad2/3-pathway ligands—potentially activins, GDF8, and GDF11 in combination—plays a role in RAP-011-induced reversal of vascular remodeling and improvement in hemodynamic and cardiac structural parameters in experimental PH.

### Example 13: Therapeutic RAP-011 Treatment Reverses Cardiac Remodeling in Severe Experimental PAH

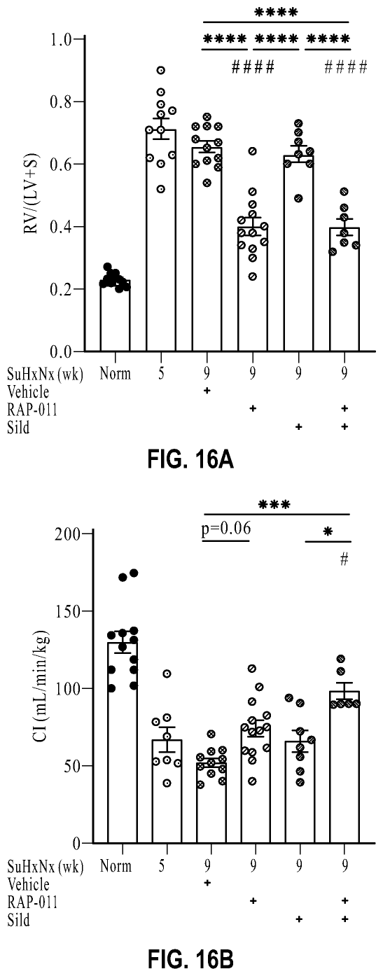

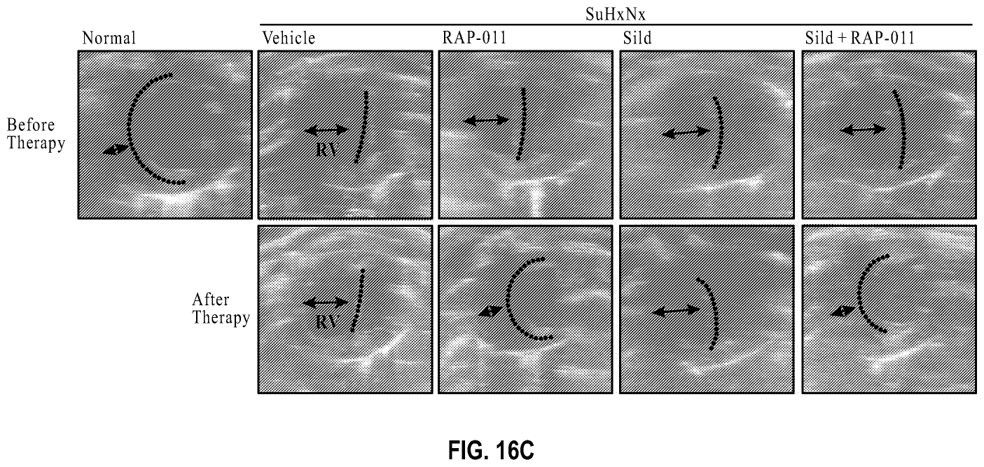

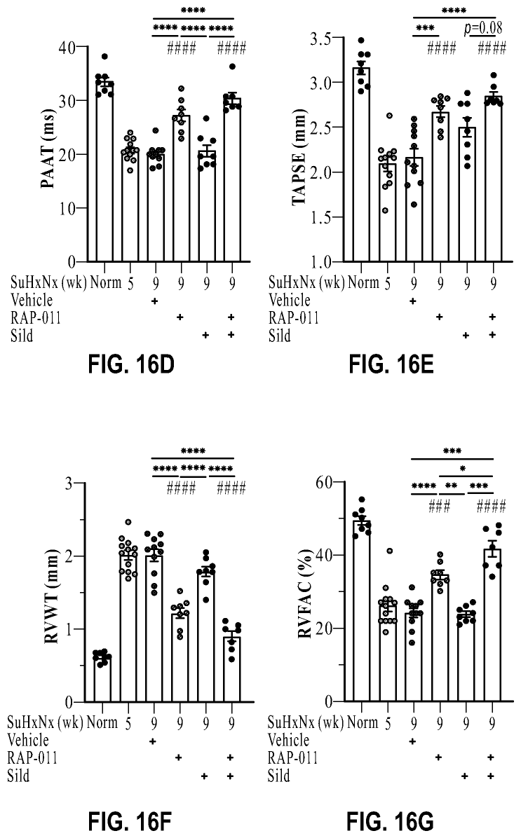

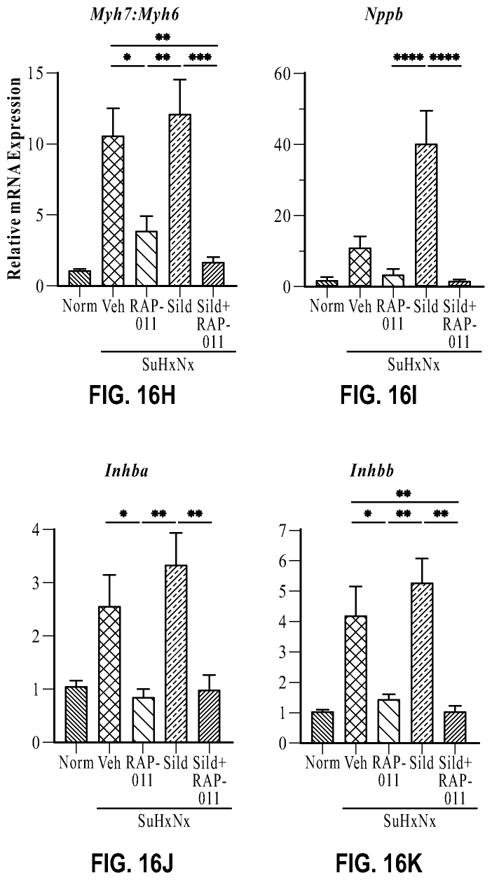

We then investigated whether RAP-011 reverses established cardiac remodeling in the therapeutic SuHxNx model of severe PAH (FIG. 16A). In untreated SuHxNx rats, we confirmed right ventricular hypertrophy and an impaired cardiac index at the onset of therapeutic treatment (week 5) that persisted undiminished through week 9 (FIG. 16A and FIG. 16B). Therapeutic treatment with RAP-011 starting 5 weeks after disease initiation significantly improved these parameters by week 9 compared to untreated SuHxNx rats at the same time point (FIG. 16A and FIG. 16B). RAP-011 treatment produced improvement superior to that of sildenafil and, importantly, normalized these parameters more effectively when combined with sildenafil than sildenafil alone (FIG. 16A and FIG. 16B).

We next used echocardiography to investigate effects of therapeutic treatment with RAP-011 on cardiac parameters in this model of severe PAH. Echocardiographic assessments conducted at week 5 (before therapy) and week 9 (after therapy) in each rat demonstrated that RAP-011 treatment—either alone or in combination with sildenafil—reversed right ventricular dilatation and septal wall flattening whereas sildenafil alone did not (FIG. 16C). As determined by echocardiography, untreated SuHxNx rats developed abnormalities in pulmonary artery acceleration time (PAAT), TAPSE, right ventricular wall thickness (RVWT), and right ventricular fractional change (RVFC) by the onset of therapeutic treatment (week 5) that were maintained through week 9 (FIG. 16D, FIG. 16E, FIG. 16F, and FIG. 16G). Therapeutic treatment with RAP-011 starting 5 weeks after disease initiation markedly improved these parameters by 9 weeks compared to untreated SuHxNx rats at the same time point (FIG. 16D, FIG. 16E, FIG. 16F, and FIG. 16G). Except for the case of TAPSE, RAP-011 treatment produced improvement superior to that of sildenafil and normalized each parameter more effectively when combined with sildenafil than sildenafil alone (FIG. 16D, FIG. 16E, FIG. 16F, and FIG. 16G).

We also examined effects of therapeutic treatment with RAP-011 on selected markers of cardiac dysfunction in the SuHxNx model of severe PAH. Cardiac remodeling and heart failure are associated with a shift in myosin heavy chain isoform from a to P (increased Myh7:Myh6 ratio), increased levels of natriuretic peptide B (Nppb), and increased activin-ActRII signaling (33-35) (Krenz 2004, Kerkela 2015; Roh 2019). As compared with normal, right ventricular tissue from vehicle-treated SuHxNx rats at week 9 displayed an increased ratio of Myh7:Myh6 expression, increased Nppb expression, and increased expression of β-subunits for activin A (Inhba) and activin B (Inhbb) (FIG. 16H, FIG. 16I, FIG. 16J, and FIG. 16K). In each case, therapeutic treatment with RAP-011 partially or fully normalized expression of these markers compared to vehicle whereas therapeutic treatment with sildenafil did not (FIG. 16H, FIG. 16I, FIG. 16J, and FIG. 16K). Importantly, combination of RAP-011 with sildenafil also normalized expression of these markers more effectively than sildenafil alone (FIG. 16H, FIG. 16I, FIG. 16J, and FIG. 16K). Together, these results demonstrate that RAP-011 monotherapy and combination therapy incorporating RAP-011 reverse cardiac remodeling in severe experimental PAH.

### Example 14: RAP-011 Exerts Cardioprotective Effects in a Model of Pressure Overload

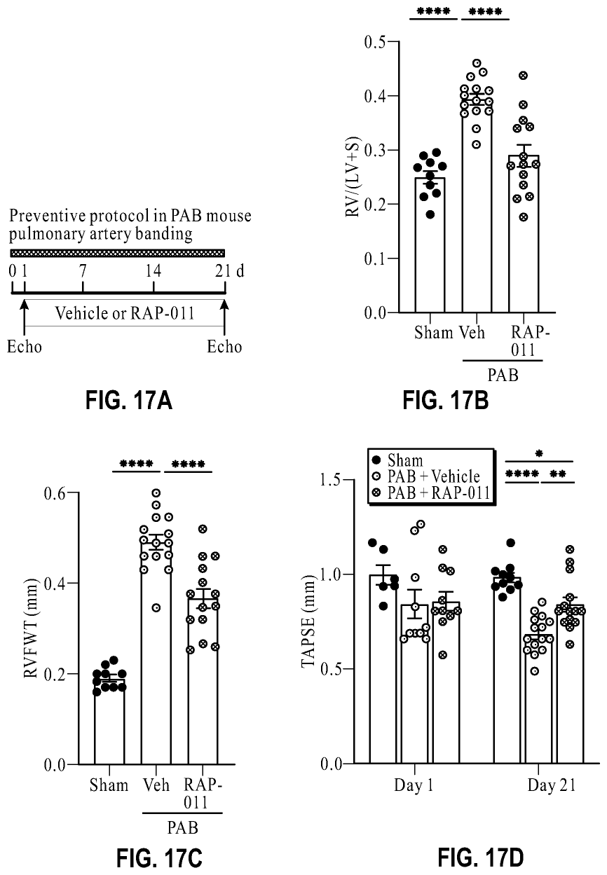

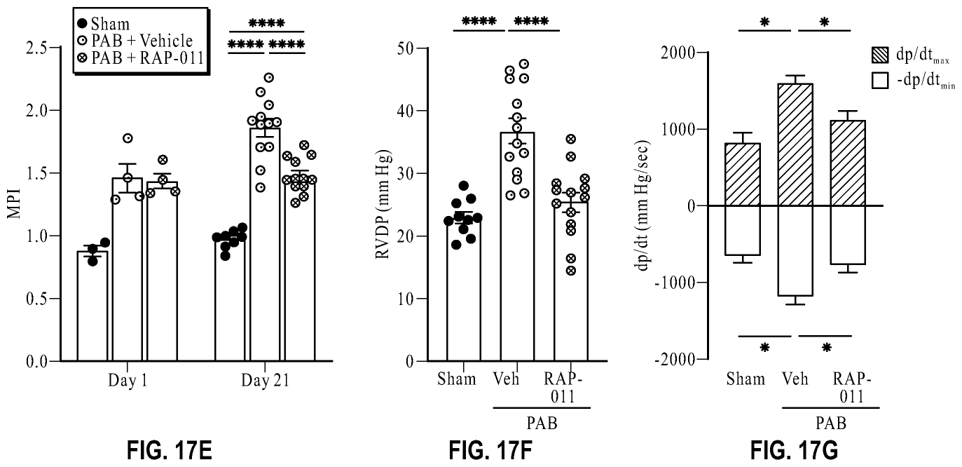

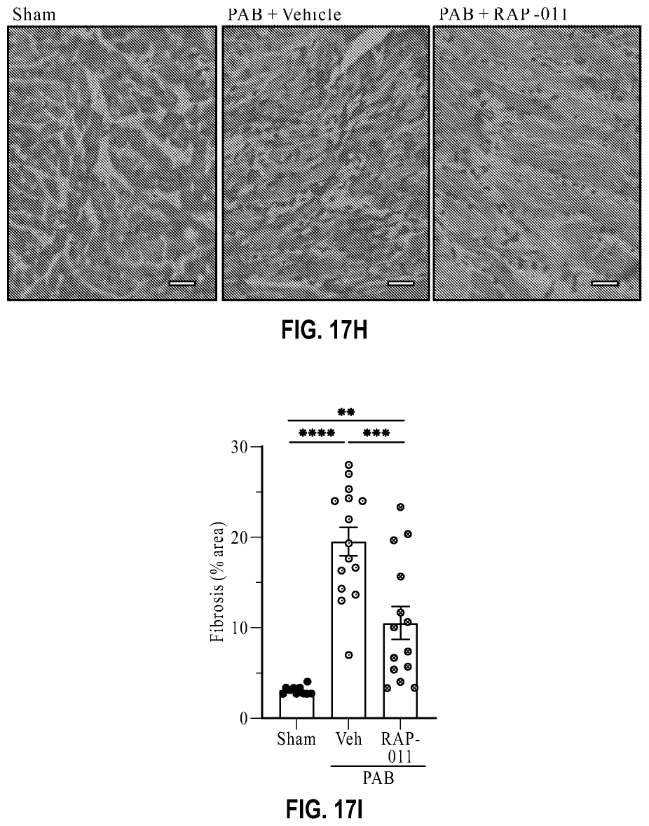

To determine whether the foregoing cardioprotective actions of RAP-011 may be due to direct effects on the heart, we next investigated cardiac effects of RAP-011 treatment under conditions of continuous pressure overload in mice subjected to pulmonary artery banding (PAB) to simulate chronic elevation of pulmonary vascular resistance (FIG. 17A). Compared to sham procedure, PAB produced right ventricular hypertrophy and dysfunction by study end at day 21, while treatment with RAP-011 significantly reduced these PAB-induced changes (FIG. 17B, FIG. 17C, FIG. 17D, and FIG. 17E). As determined by right ventricular catheterization at study end, developed pressure and absolute values for peak rates of pressure change in the right ventricle were increased by PAB and fully or partially normalized by RAP-011 treatment (FIG. 17F and FIG. 17G). These functional improvements were accompanied by significantly reduced fibrosis in the right ventricle of RAP-011-treated mice compared to vehicle (FIG. 17H and FIG. 17I). Together, these results indicate that RAP-011 confers structural, functional, and histologic benefits on the right ventricle under conditions of continuous pressure load, thereby implicating direct cardioprotective actions of RAP-011 as an important component of its therapeutic effects in severe experimental PAH.

### Example 15: Persistence of RAP-011-Induced Cardiopulmonary Benefits in Severe Experimental PAH

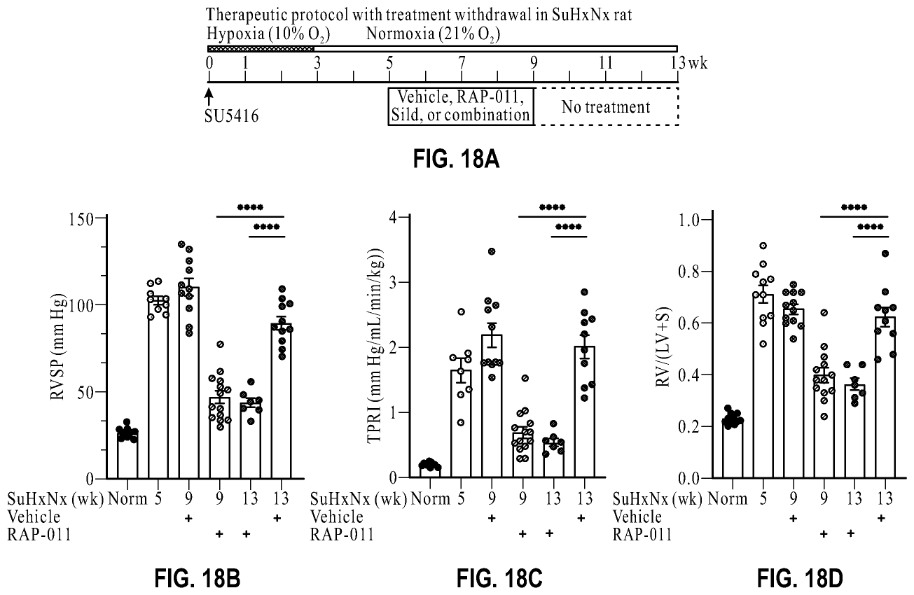

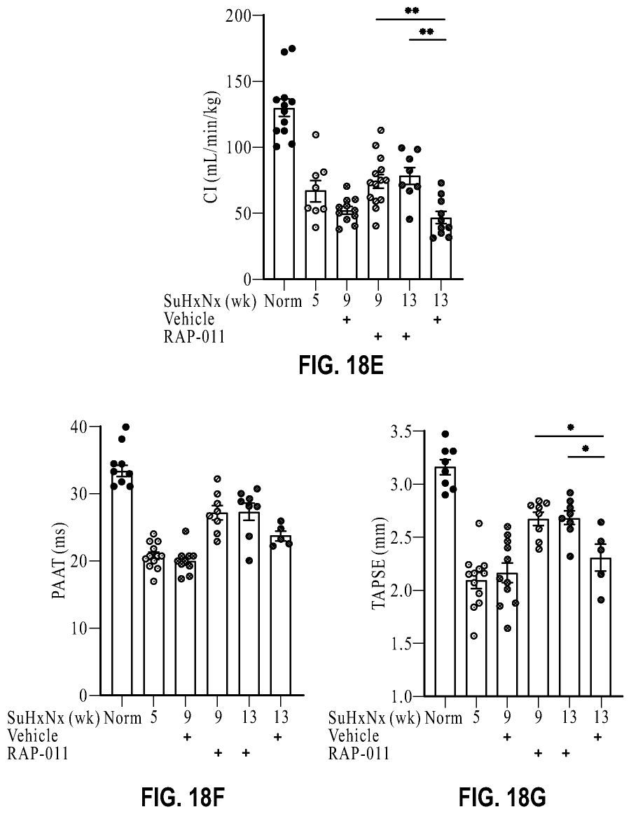

Finally, we investigated whether cardiopulmonary benefits of therapeutic RAP-Oll treatment in severe experimental PAH are sustained after treatment cessation (FIG. 18A). In untreated SuHxNx rats, we confirmed that structural and functional abnormalities present by week 5 remain largely undiminished through week 13 (FIG. 18B, FIG. 18C, FIG. 18D, FIG. 18E, FIG. 18F, and FIG. 18G). These endpoints include right ventricular systolic pressure, total pulmonary vascular resistance, right ventricular hypertrophy, cardiac index, pulmonary artery acceleration time, and tricuspid annular plane of systolic excursion. Therapeutic treatment with RAP-011 starting at week 5 produced significant improvement in these parameters by week 9 compared to untreated SuHxNx rats at the same time point (FIG. 18B, FIG. 18C, FIG. 18D, FIG. 18E, FIG. 18F, and FIG. 18G). Importantly, SuHxNx rats treated therapeutically with RAP-011 from weeks 5 to 9 displayed persistence of significant improvements in each of these endpoints 4 weeks after treatment withdrawal (FIG. 18B, FIG. 18C, FIG. 18D, FIG. 18E, FIG. 18F, and FIG. 18G). Circulating levels of RAP-011 were undetectable by 2 weeks after treatment withdrawal (data not shown). These results indicate that RAP-011-induced reversal of cardiopulmonary remodeling in severe experimental PAH is sustained for at least a month after treatment cessation.

### Example 16: Effects of Preventive ActRIIA-mFc Treatment in Models of Acquired and Genetic PH

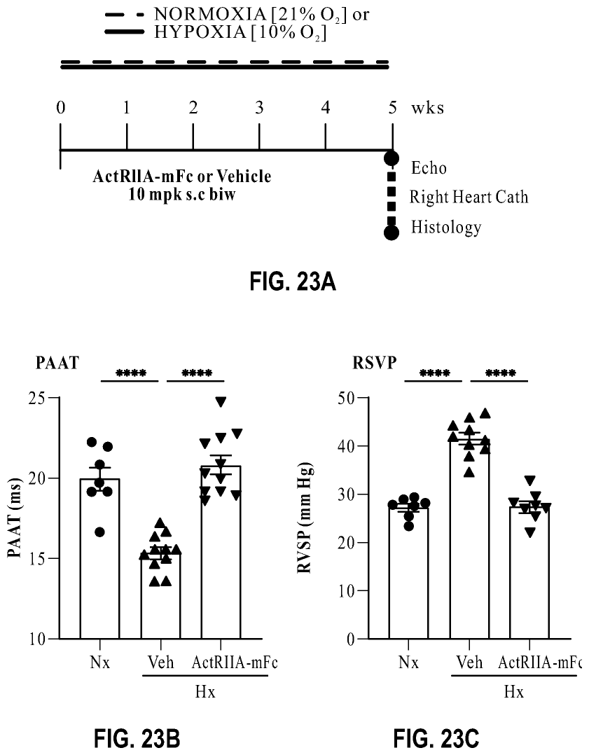

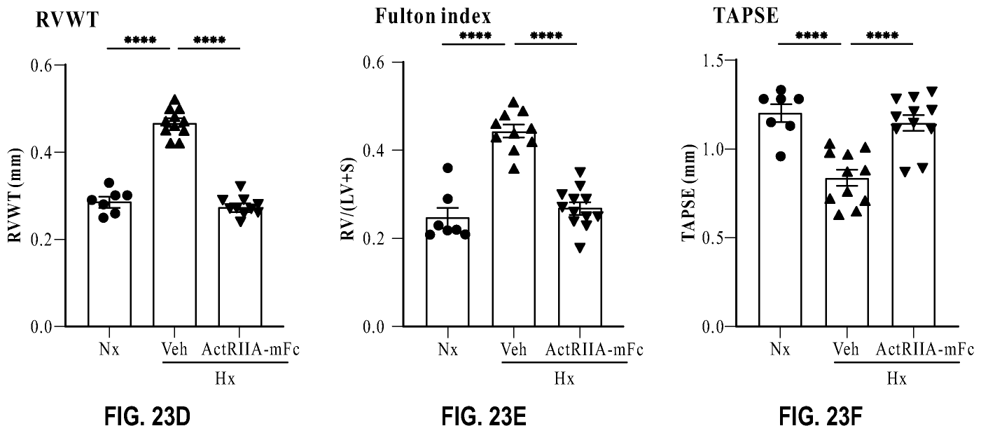

The effects of an ActRIIA-mFc fusion protein (ActRIIA-mFc homodimer as described in Example 1) was examined in a mouse model of PH caused by Bmpr2 haploinsufficiency since the BMPR2 pathway is heavily implicated in pulmonary vascular homeostasis and familial PAH. In this model, Brnpr2^{+/R899X} mice were exposed to hypoxic conditions for 5 weeks starting at 4 months of age (FIG. 23A). See, e.g., Long L, et al. Nat Med. 2015; 21(7):777-785.

Twenty-nine Bmpr2^{+/R99X} mice were randomized into three groups: (i) seven mice were housed in normoxic conditions for 5 weeks, “Nx”; (ii) eleven mice were housed in hypoxic conditions and injected subcutaneously with vehicle control (phosphate buffered saline (PBS)), twice weekly for 5 weeks, “Hx Veh”; and (iii) eleven mice were housed in hypoxic conditions and injected subcutaneously with ActRIIA-mFc at a dose of 10 mg/kg twice weekly for 5 weeks, “Hx ActRIIA-mFc.” At the end of the study, echocardiography and pressure-volume catheter were performed to measure left and right ventricular remodeling and functional changes before animals were euthanized for heart and lung collection. Heart and lungs of each mouse were weighed.

Prior to euthanasia, in vivo cardiac function was assessed by transthoracic echocardiography (Vevo 3100, VisualSonics, Toronto, ON, Canada) in conscious mice. RV systolic pressure (RVSP) was measured by advancing a curved-tip pressure transducer catheter (SPR-1000, Millar Instruments) into the RV via the right jugular vein. Right ventricular free wall thickness (RVWT) was measured using M-mode in a modified parasternal long-axis view through the aortic valve. Tricuspid annular plane systolic excursion (TAPSE) was obtained from the apical four-chamber view directing the M-mode doppler beam through the lateral annulus of the tricuspid valve plane. Pulmonary artery acceleration time (PAAT) was measured as the time from start to peak velocity of blood flow in the lumen of the main pulmonary artery distal to the pulmonary valve as obtained from the pulse-wave doppler recording. RV hypertrophy was determined by calculating the weight ratio of the RV free wall to the combined left ventricle and septum (Fulton index). Macrophage infiltration was assessed by immunohistochemical staining for macrophage marker F4/80. Percentage of F4/80-positive cells in lung based on assessment of 40 high-magnification fields per animal.

Compared to normoxic controls, mice exposed to hypoxic conditions had significantly decreased pulmonary artery acceleration time (FIG. 23B), elevated right ventricular systolic pressure (FIG. 23C), increased right ventricular free wall thickness (FIG. 23D), induced right ventricular hypertrophy (FIG. 23E), and decreased the tricuspid annular plane systolic excursion (FIG. 23F). Each of these parameters was normalized with preventive treatment with ActRIIA-mFc (10 mpk twice weekly as a subcutaneous injection).

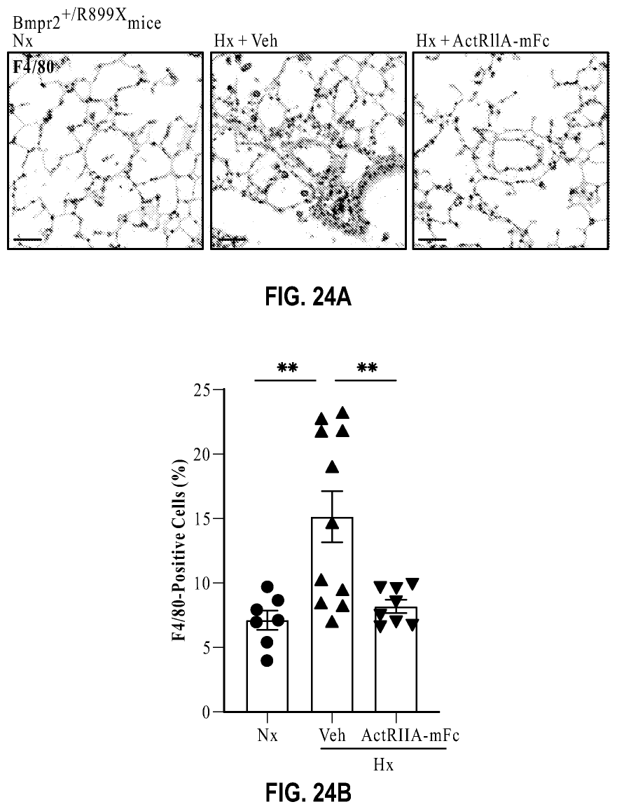

Post-mortem analysis of macrophage infiltration in the lung showed that treatment with an ActRIIA-mFc fusion protein prevented perivascular inflammation by preventing macrophage infiltration in the lung (FIG. 24A and FIG. 24B). Together, these results indicate that preventive treatment with ActRIIA-mFc produces strong beneficial effects not only in widely-used models of acquired PH but also in a model of heritable PH caused by Bmpr2 haploinsufficiency.

1-212. (canceled)

213. A method of treating pulmonary arterial hypertension (PAH) in a patient in need thereof, comprising administering a therapeutically effective amount of an ActRIIA fusion protein comprising:
a) an ActRIIA polypeptide comprising an amino acid sequence of SEQ ID NO: 2;
b) an Fc domain comprising an amino acid sequence that is at least 95% identical to SEQ ID NO: 32; and
c) a linker domain, positioned between the ActRIIA polypeptide and the Fc domain;
wherein administration of the ActRIIA fusion protein results in one or more of the following:
a) reduction of the risk of death by at least 50%;
b) decrease of N-terminal pro B-type natriuretic peptide (NT-proBNP) levels by at least 400 μg/mL;
c) reduction of the mean right atrial pressure (mRAP) in the patient by at least 10%;
d) improvement in right ventricular function due to an increase in right ventricular fractional area change, a decrease in right ventricular hypertrophy, and/or an increase in ejection fraction;
e) reduction in pulmonary vascular resistance (PVR) from baseline to 24 weeks by at least 1 Wood Unit;
f) increase in 6-minute walk distance (6 MWD) from baseline to 24 weeks by at least 25 meters; or
g) prevents or reduces pulmonary hypertension Functional Class progression as recognized by the World Health Organization (WHO).

214. The method of claim 213, wherein the Fc domain comprises an amino acid sequence that is at least 99% identical to the amino acid sequence of SEQ ID NO: 32.

215. The method of claim 213, wherein the Fc domain comprises the amino acid sequence of SEQ ID NO: 32.

216. The method of claim 214, wherein the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21).

217. The method of claim 214, wherein the linker domain is TGGG (SEQ ID NO: 20).

218. A method of claim 213, wherein the ActRIIA fusion protein comprised an amino acid sequence that is at least 95% identical to the amino acid sequence of SEQ ID NOs: 23 or 41.

219. The method of claim 218, wherein the fusion protein comprises an amino acid sequence that is at least 99% identical to the amino acid sequence of SEQ ID NO: 23.

220. The method of claim 219, wherein the fusion protein comprises the amino acid sequence of SEQ ID NO: 23.

221. The method of claim 218, wherein the fusion protein comprises an amino acid sequence that is at least 99% identical to the amino acid sequence of SEQ ID NO: 41.

222. The method of claim 221, wherein the fusion protein comprises the amino acid sequence of SEQ ID NO: 41.

223. The method of claim 217, wherein the polypeptide is part of a homodimer protein complex.

224. The method of claim 217, wherein the polypeptide is glycosylated.

225. The method of claim 217, wherein the polypeptide binds to one or more ligands selected from the group consisting of: activin A, activin B, GDF11, BMP10, GDF8, and BMP6.

226. The method of claim 217, wherein administration of the fusion protein increases the patient's 6 MWD by at least 25 meters.

227. The method of claim 217, wherein administration of the fusion protein increases the patient's 6 MWD by at least 30 meters.

228. The method of claim 217, wherein administration of the fusion protein increases the patient's 6 MWD by at least 40 meters.

229. The method of claim 217, wherein administration of the fusion protein reduces the PVR from baseline to 24 weeks by at least 1 Wood Unit.

230. The method of claim 217, wherein administration of the fusion protein reduces the PVR by at least 2 Wood Units.

231. The method of claim 217, wherein administration of the fusion protein reduces the PVR by at least 4 Wood Units.

232. The method of claim 217, wherein administration of the fusion protein decreases N-terminal pro B-type natriuretic peptide (NT-proBNP) levels by at least 400 pg/mL.

233. The method of claim 217, wherein administration of the fusion protein reduces the mean right atrial pressure (mRAP) in the patient by at least 10%.

234. The method of claim 217, wherein administration of the fusion protein delays clinical worsening of pulmonary arterial hypertension in accordance with the World Health Organization's functional classification system for pulmonary hypertension.

235. The method of claim 217, wherein administration of the fusion protein reduces the risk of hospitalization for one or more complications associated with pulmonary arterial hypertension.

236. The method of claim 217, wherein administration of the fusion protein reduces the risk of death by at least 50%.

237. The method of claim 217, wherein the fusion protein is administered subcutaneously.

238. The method of claim 217, wherein the fusion protein is administered at a first dose of between 0.1 mg/kg and 1.0 mg/kg and a second dose of between 0.1 mg/kg and 1.0 mg/kg of said fusion protein, wherein the second dose is administered three weeks after the first dose.

239. The method of claim 238, wherein the first dose of the ActRIIA fusion protein is administered at a dose of 0.3 mg/kg.

240. The method of claim 238, wherein the second dose of the ActRIIA fusion protein is administered at a dose of 0.7 mg/kg.

241. The method of claim 213, comprising further administering to the patient an additional active agent and/or supportive therapy.

242. A kit comprising a lyophilized polypeptide and an injection device, wherein the lyophilized polypeptide is an ActRIIA fusion protein comprising:
a) an ActRIIA polypeptide comprising an amino acid sequence that is at least 90% identical to an amino acid sequence corresponding to residues 30-110 of SEQ ID NO: 1.
b) an Fc domain of an IgG1 immunoglobulin; and
c) a linker domain,
wherein the linker domain is positioned between the ActRIIA polypeptide and the Fc domain of the IgG1 immunoglobulin,
wherein the kit comprises one or more vials containing between 25 mg to 60 mg of the lyophilized ActRIIA fusion protein, citric acid monohydrate, tri-sodium citrate, polysorbate 80, and sucrose.


SEQUENCE LISTING (44 sequences)

SEQ ID NO: 1 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 2 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 3 - protein, 100 aa, synthetic construct
  REGION 1..100: note = Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 4 - DNA, 1539 bp, Homo sapiens
  source 1..1539: mol_type = unassigned DNA
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 5 - DNA, 345 bp, Homo sapiens
  source 1..345: mol_type = unassigned DNA
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 6 - protein, 115 aa, Homo sapiens; identical to SEQ ID NO: 2

SEQ ID NO: 7 - protein, 115 aa, Ovis aries
ILGRSETQECIFYNANWERDRTNRTGVESCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 8 - protein, 115 aa, unidentified
  REGION 1..115: note = Description of Unknown: Mole ActRIIA sequence
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKAP

SEQ ID NO: 9 - protein, 115 aa, Mus sp.
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKPP

SEQ ID NO: 10 - protein, 115 aa, Gallus gallus
ILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKPP

SEQ ID NO: 11 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 14 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 15 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - skipped

SEQ ID NO: 17 - protein, 4 aa, synthetic construct
  REGION 1..4: note = Description of Artificial Sequence: Synthetic peptide
GGGG

SEQ ID NO: 18 - protein, 5 aa, synthetic construct
  REGION 1..5: note = Description of Artificial Sequence: Synthetic peptide
TGGGG

SEQ ID NO: 19 - protein, 5 aa, synthetic construct
  REGION 1..5: note = Description of Artificial Sequence: Synthetic peptide
SGGGG

SEQ ID NO: 20 - protein, 4 aa, synthetic construct
  REGION 1..4: note = Description of Artificial Sequence: Synthetic peptide
TGGG

SEQ ID NO: 21 - protein, 4 aa, synthetic construct
  REGION 1..4: note = Description of Artificial Sequence: Synthetic peptide
SGGG

SEQ ID NO: 22 - protein, 5 aa, synthetic construct
  REGION 1..5: note = Description of Artificial Sequence: Synthetic peptide
GGGGS

SEQ ID NO: 23 - protein, 344 aa, synthetic construct
  REGION 1..344: note = Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 21 aa, Apis sp.
MKFLVNVALVFMVVYISYIYA

SEQ ID NO: 25 - protein, 22 aa, Homo sapiens
MDAMKRGLCCVLLLCGAVFVSP

SEQ ID NO: 26 - protein, 20 aa, unidentified
  REGION 1..20: note = Description of Unknown: Activin receptor type-2A sequence
MGAAAKLAFAVFLISCSSGA

SEQ ID NO: 27 - protein, 369 aa, synthetic construct
  REGION 1..369: note = Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 1114 bp, synthetic construct
  misc_feature 1..1114: note = Description of Artificial Sequence: Synthetic polynucleotide
  source 1..1114: mol_type = other DNA
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 29 - protein, 9 aa, synthetic construct
  REGION 1..9: note = Description of Artificial Sequence: Synthetic peptide
ILGRSETQE

SEQ ID NO: 30 - protein, 329 aa, synthetic construct
  REGION 1..329: note = Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 32 - protein, 225 aa, synthetic construct; identical to SEQ ID NO: 11
  REGION 1..225: note = Description of Artificial Sequence: Synthetic polypeptide

SEQ ID NO: 33 - protein, 223 aa, synthetic construct; identical to SEQ ID NO: 12
  REGION 1..223: note = Description of Artificial Sequence: Synthetic polypeptide

SEQ ID NO: 34 - protein, 232 aa, synthetic construct; identical to SEQ ID NO: 13
  REGION 1..232: note = Description of Artificial Sequence: Synthetic polypeptide

SEQ ID NO: 35 - protein, 229 aa, synthetic construct; identical to SEQ ID NO: 15
  REGION 1..229: note = Description of Artificial Sequence: Synthetic polypeptide

SEQ ID NO: 36 - protein, 115 aa, Bos taurus; identical to SEQ ID NO: 7

SEQ ID NO: 37 - protein, 115 aa, Tyto alba; identical to SEQ ID NO: 10

SEQ ID NO: 38 - protein, 115 aa, Myotis davidii
ILGRSETQECIFYNANWERDKTNRTGVELCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 39-40 - skipped (2 entries)

SEQ ID NO: 41 - protein, 343 aa, synthetic construct
  REGION 1..343: note = Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 6 aa, synthetic construct
  REGION 1..6: note = Description of Artificial Sequence: Synthetic 6xHis tag
HHHHHH

SEQ ID NO: 43 - DNA, 12 bp, synthetic construct
  misc_feature 1..12: note = Description of Artificial Sequence: Synthetic oligonucleotide
  source 1..12: mol_type = other DNA
ggccgaacaaat

SEQ ID NO: 44 - DNA, 12 bp, synthetic construct
  misc_feature 1..12: note = Description of Artificial Sequence: Synthetic oligonucleotide
  source 1..12: mol_type = other DNA
ggcygaacaaat